(19) United States
(12) Patent Application Publication
(10) Pub. No.: US 2005/0026263 A1
(43) Pub. Date: Feb. 3, 2005

(54) Multi-functional antibodies

(75) Inventors: Claude F. Meares, Davis, CA (US); Todd M. Corneillie, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Oakland, CA (US)

(21) Appl. No.: 10/631,258

(22) Filed: Jul. 31, 2003

(51) Int. Cl. C12N 9/00; C07K 16/46

(52) U.S. Cl. 435/188.5; 530/391.1

(57) ABSTRACT

The present invention provides antibodies comprising an antigen recognition domain that specifically binds to a metal chelate: mutant antibodies comprising a reactive site not present in the wild-type of the antibody, wherein the reactive site is in a position proximate to or within the antigen recognition domain; and methods of using such antibodies to diagnose and treat disease.

### CROSS-REFERENCES TO RELATED APPLICATIONS

This application is a continuation-in-part of U.S. patent application Ser. No. ______, filed Jul. 22, 2003 (Attorney Docket No. 023070-130910US, Multi-Functional Antibodies) which is a continuation-in-part U.S. patent application Ser. No. 10/350555, filed Jan. 23, 2003, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under Grant No. CA16861, awarded by the NIH/NCI to C. F. Meares. The Government has certain rights in this invention.

### BACKGROUND OF THE INVENTION

Over a million new cases of cancer will be diagnosed this year in the United States. While surgery can often provide definitive treatment of cancer in its early stages, the eradication of metastases is crucial to the cure of more advanced disease. Chemotherapeutic drugs are used in combinations for this purpose, with considerable success. Nonetheless, over half a million Americans will die from cancer this year. Progressions and relapses following surgery and chemotherapy/radiation are not uncommon, and in most cases the second line of treatment is of limited use. Despite the expenditure of large amounts of public and private resources over many years, better treatments for cancer are sorely needed.

Currently there are approximately 100 antineoplastic drugs on the market. Their systemic use is associated with undesirable side effects including toxicity to normal cells, which limits the doses used for treatment of the disease. Most pharmaceuticals consist of small organic molecules, which effectively traverse cell membranes and become widely distributed through the body. As reviewed by Langer, polymer-based pharmaceutical agents provide a variety of new approaches to safer and better therapies (see, Langer R, Nature, 392 (6679) SUPPS: 5-10 (1998)). Polymers and other macromolecules do not traverse membranes; however, they may be selectively accumulated in the interstitial space of a tumor, since tumors typically do not possess an efficient lymphatic drainage system (Yuan et al., Cancer Research 51(12): 3119-30 (1991)). Developing technology to target therapeutic drugs to cancer cells, while sparing normal cells, is a promising approach to improved treatment; visualizing small cancers by means of targeting reagents is already a productive area of investigation.

The residence of macromolecules in tumors may be prolonged if they become anchored to immobile elements, such as polymorphic epithelial mucin (PEM), the secreted product of the MUCI gene (Taylor-Papadimitriou et al., Trends Biotechnol., 12(6): 227-33 (1994)); or HLA-DR, a long-lived cell surface protein (Rose et al., Cancer Immunology Immunotherapy, 43: 26-30 (1996). The reagents of choice for this anchoring reaction are monoclonal antibodies and their derivatives. Currently there is a good selection of such macromolecules that bind to highly expressed tumor antigens, and that do not bind significantly to normal cells. Examples include, HMFG1 (Nicholson et al., Oncology Reports 5: 223-226 (1998)); L6 (DeNardo et al., Journal of Nuclear Medicine 39: 842-849 (1998)); and Lym-1 (DeNardo et al., Clinical Cancer Research, 3: 71-79 (1997)). The latter three antibodies have been conjugated to metal chelates for radioimmunotherapy and studied extensively in recent years, and are in clinical trials at various stages.

Recent data indicate that immunoconjugates have efficacy comparable to conventional antineoplastic drugs, and work in synergy with them (see, for example, Nicholson et al., Oncology Reports 5: 223-226 (1998); and DeNardo et al., Proceedings of the National Academy of Sciences USA 94: 4000-4004 (1997)). The emerging success of metal radioimmunoconjugates for cancer detection and treatment owes much to the development of metal-binding molecules (bifunctional chelating agents) appropriate for use in vivo, and to the further development of linkers that reduce concentrations of the metal binding molecules in nontarget tissues (see, Sundberg et al., Nature 250: 587-588 (1974); Yeh et al., Analytical Biochemistry 100: 152-159 (1979); Moi et al., Analytical Biochemistry 148: 249-253 (1985); Moi et al., Journal of the American Chemical Society 110: 6266-6267 (1988); and Li et al., Bioconjugate Chemistry 4: 275-283 (1993).

An alternative view of the potential for use of antibodies in cancer diagnosis and therapy is that, rather than carrying a radionuclide to a tumor, they can carry a receptor for a radionuclide (e.g., for a metal chelate). Antibodies against metal chelates can bind target molecules with high affinity and exquisite specificity and thus, can conveniently be used for applications in chemistry, environmental science, and medicine (see, e.g., Reardan et al., Nature 316: 265-268 (1985); Barbas et al., Proc. Natl. Acad. Sci. U.S.A. 90: 6385-6389 (1993); Blake et al., J. Biol. Chem. 271: 27677-27685 (1996); Brummer et al., Bioorg. Med. Chem. 9: 2253-2257 (2001); Schuhmacher et al., Cancer Res. 61: 3712-3717 (2001); Johnson et al., Environ. Sci. Technol. 36: 1042-1047 (2002)). For example, an antibody that binds rare earth complexes selectively could be used as a docking station for a set of probe molecules, of particular interest for medical imaging and therapy (see, e.g., Lubic et al., J. Nucl. Med. 42: 670-678 (2001) and Bosslet et al., Br. J. Cancer 63: 681-686 (1991)). The rare earths are rich in probe properties, such as the paramagnetism of Gd, the luminescence of Tb and Eu, and the nuclear properties of Y and Lu. The chelating ligand DOTA (1,4,7,10-tetraazacyclododecane-N,N′,N″,N′″-tetraacetic acid) is generally useful for binding transition metals and rare earths with extreme stability under physiological conditions, leading to their use in vivo for targeted imaging and therapy (see, e.g., Loncin et al., Inorg. Chem. 25: 2646-2648 (1986); Geraldes et al., Magn. Reson. Med. 3: 242-250 (1986); Wu et al., Proc. Natl. Acad. Sci. U.S.A. 97: 8495-8500 (2000); and Waldherr et al., J. Nucl. Med. 43: 610-616 (2002)).

Antibodies with dual binding specificity have been prepared which can, e.g., cross-link tumor cells to cytokines such as tumor necrosis factor (Bruno et al., Cancer Res. 56(20): 4758-4765 (1996)). Likewise, bispecific antibodies that can bind to tumors and to metal chelates have been developed (Stickney et al., Cancer Res. 51(24): 6650-5 (1991); Rouvier et al., Horm. Res. 47(4-6): 163-167 (1997)). When pretargeted to tumors, these bispecific antibodies bind to antigens and remain on the target, providing receptors for metal chelates. Subsequent administration of small, hydrophilic metal chelates leads to their capture by the targeted chelate receptors. Uncaptured chelates clear quickly through the kidneys and out of the body, leaving very little radioactivity in normal tissues. This strategy is known as “pretargeting.”

A triumph of this approach was the imaging of metastatic cancer in the liver by an indium-111 chelate (Stickney et al., Cancer Res. B(24): 6650-5 (1991)). Antibodies conventionally conjugated to metal chelates are catabolized in the liver, and generally produce a radioactive background that masks tumors in that organ. The excellent tumor-to-background uptake ratios achieved by the pretargeting approach have led to considerable exploration of improvements in methodology. The anti-chelate antibody CHA255, initially developed for this purpose, possesses a high binding constant for (S)-benzyl-EDTA-indium chelates (Kₛ≈4×10⁹) and exquisite specificity for these haptens (Dayton et al., Nature 316: 265-268 (1985). On CHA255, the bound lifetimes of various indium chelates at 37° C. were found to be in the 10-40 min range (Meyer, et al, Bioconjugate Chem. 1(4): 278-84 (1990)).

Another anti-chelate antibody, 2D12.5, possesses a high binding constant for (S) nitrobenzyl-DOTA chelates and Janus-DOTA, a bivalent form of DOTA (Kₛ≈10⁸) (Goodwin et al., Canc. Res. 54(22): 5937-46 (1994) and Lubic et al., J. Nucl. Med. 42(4): 670-78 (2001)). In vivo clearance studies have demonstrated that 74-96% of 2D12.5 bound to metal chelates is present 24 hours after administration of the bound complex.

The need to enhance the antibody-hapten bound lifetime has led to the use of the long-lived avidin-biotin interaction, employing biotinylated metal chelates (Chinol et al., Nuclear Medicine Communications 18: 176-182 (1997)) in place of the original antibody-hapten interaction between CHA255 and benzyl-EDTA-indium derivatives. Here one assembles an antibody-avidin-chelate complex at the target in two or three steps, by sequential administration of nonradiolabeled proteins with a final administration of a biotinyl chelate carrying a radiometal. The extremely high affinity biotin-avidin association is adequately long-lived even for therapeutic applications (Theodore L J. et al, WO 9515979). Hen egg avidin and bacterial streptavidin, however, are both nonhuman, tetrameric proteins: their immunogenic properties are inconvenient, and the reversible associations between their subunits may limit their effectiveness. Thus, an improved strategy is still needed.

A delivery strategy based on the formation of a covalent bond between a chelate and an antibody that specifically recognizes and binds the chelate would represent a significant improvement over the methods now in use. The present invention provides engineered antibodies and chelates that react with one another to form covalent bonds and methods of using the engineered constructs to perform analyses and treat diseases.

### SUMMARY OF THE INVENTION

An object of the present invention is the engineering of antibodies that recognize chelating agents and metal chelates, particularly macrocyclic metal chelates. The antibodies of the invention are desirable for use as analytical reagents and in diagnosis and therapy. For example, certain antibodies of the invention recognize and bind chelates formed between a metal ion that is a radionuclide and a chelating agent. The decay of the radionuclide is utilized to locate a region of disease, e.g., a tumor or to treat the disease through radiotherapy.

Thus, in a first aspect there is provided an antibody that has an antigen recognition domain that recognizes a macrocylic metal chelate. The macrocyclic metal chelate preferably includes four heteroatoms (e.g., O, S, N and combinations thereof) within its structure. Exemplary macrocyclic chelates include four nitrogen atoms. Additional macrocycles and methods of making macrocycle derivatives are well known in the art and described in, e.g., Synthesis of Macrocycles: the Design of Selective Complexing Agents (Izatt and Christensen ed., 1987) and The Chemistry of Macrocyclic Ligand Complexes (Lindoy, 1989). One of skill in the art will appreciate that the metal ion may be any lanthanide, actinide, group IIIb transition metal, alkaline earth metal, or metal so long as X does not substantially affect the binding affinity of the antibody and the metal chelate. Typically, the lanthanide is La, Ce, Pr, Nd, Sm, Eu, Gd, Tb, Dy, Ho, Er, Ym, Yb, Lu, or Pm. Typically, the actinide is Ac, Pa, or Am. Typically, the group Imb transition metal is Y or Sc. Typically, the alkaline earth metal is Sr. Typically, the metal is In, Ti, or Bi. In addition, R² may comprise an amino acid side chain, e.g., Lys, Cys, Glu, or Asp.

In an exemplary embodiment,the V_{L} chain of the antibody is not encoded by the nucleic acid sequence set forth in SEQ ID NO:1 and the V_{H} chain of the antibody is not encoded by the nucleic acid sequence set forth in SEQ ID NO:5.

In a preferred embodiment, the antibody recognizes and binds to an array of macrocyclic metal chelates that are structurally distinct. The “promiscuity” of the antibodies of this embodiment is a unique feature of the present invention, allowing for the design of different diagnostic and treatment regimens using a single antibody or a small cohort of antibodies.

In another aspect, the invention provides a mutant antibody comprising a reactive site not present in the wildtype of said antibody and an antigen recognition domain that recognizes a macrocyclic metal chelate comprising four nitrogen atoms, wherein said reactive site is in a position proximate to or within said antigen recognition domain. The presence of the complementary reactive functional groups on the antibody and the chelate allow for the formation of a covalent bond between the antibody and the chelate, essentially forming an irreversible adduct between the antibody and chelate. By varying the pendant reactive functional group present on a chelate and/or antibody it is possible to prepare a library of chelate/antibody pairs that include functional groups exhibiting a range of reactivities. The chelate-antibody pairs of the invention are useful as analytical agents and in clinical diagnosis and therapy. When the chelate-antibody pairs are used as a clinical therapeutic or diagnostic agent, the chelate circulates throughout the body of the patient to whom it is administered prior to reaching the targeting antibody, which has been pretargeted to a tissue or other site. To assure that a useful quantity of an administered dose of the chelate reaches the target antibody, the reactive group of the reactive group on the chelate is preferably selected such that it does not react substantially with elements of, for example, blood and plasma, but readily reacts with the complementary reactive site on the antibody following the formation of an antibody-antigen (chelate) complex.

An another aspect, the invention provides a mutant antibody wherein an N-linked glycosylation site has been removed. In a preferred embodiment, the N-linked glycosylation site at position 87 of the V_{H} of 2D12.5 has been removed by replacing the native Asn with a Asp residue. The N-linked glycosylation site present on the native antibody is inconsistently glycosylated, thereby leading to a heterogeneous population of antibodies. The loss of the glycosylation signal sequence leads to a population of homogeneous antibodies. This homogeneous population of antibodies can conveniently be used, e.g., in a composition for treatment of diseases such as cancer or autoimmune disorders.

In a second aspect, the invention provides a composition having the structure:
(Ab)_{n′}-L-T
wherein n′ is an integer from 1-10. The symbol Ab represents an antibody of the invention. The symbol L represents a chemical bond or linking group that may contain one or more functional groups; and T is a targeting moiety.

For purposes of illustration, the invention is described further by reference to an exemplary antibody-chelate pair. The description is for clarity of illustration, and is not intended to define or limit the scope of the present invention.

In an exemplary embodiment, a reactive site is incorporated into an anti-chelate antibody by engineering a cysteine at one of several locations that are near to the region of the antibody to which the chelate binds. The engineering is typically accomplished by site-directed mutagenesis of a nucleic acid encoding the wild-type of the anti-chelate antibody. The resulting mutant antibodies comprise a library of single-Cys mutants. Mutated antibodies, such as the single-Cys mutants can be prepared using methods that are now routine in the art (see, for example, Owens et al., Proceedings of the National Academy of Sciences USA 95: 6021-6026 (1998); Owens et al., Biochemistry 37: 7670-7675 (1998)). The library members are then tested against a library of electrophilic chelates, differing in structure and reactivity, to determine the best pairs for further study. As discussed above, the electrophilic chelates preferably do not react prematurely with nucleophiles normally present in the blood. The reactivity of the chelates with physiologically relevant groups is easily determined in vitro. In the present example, in which the nucleophile is the cysteine —SH group, important potentially interfering groups are, for example, thiols on glutathione and other small molecules, and cysteine in albumin (Geigy Scientific Tables Vol.3, C. Lentner, ed., Ciba-Geigy Ltd., Basel, Switzerland 1984). The mildly electrophilic groups on alkylating agents used in cancer chemotherapy (nitrogen mustards, ethyleneimine derivatives, mesylate esters, etc.) provide guidance concerning the practical limits of reactivity.

In a third aspect, the present invention provides a mutant antibody comprising a reactive cysteine residue that is not present in the wild-type of the antibody. The antibody also includes an antigen recognition domain that specifically binds to a metal chelate against which the antibody is raised. The reactive —SH of the cysteine is in a position proximate to or within the antigen recognition domain, such that the —SH group and the pendant reactive group on the antibody are able to form a covalent bond.

Because of the high local concentrations of nucleophile and electrophile in the antibody-hapten (chelate) complex, weaker electrophiles than those found on anticancer drugs are preferred. As discussed by Fersht, the effect of local concentration can be appreciated by comparing rate constants for the same chemical reaction between two separate reactants, and between two reactive groups joined by a linker (Alan Fersht, ENZYME STRUCTURE AND MECHANISM, 2nd Ed., Freeman, New York, 1985, pp. 56-63). The effect of high local concentration is displayed schematically in Scheme 1: 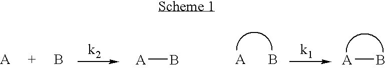
in which effective local concentration of A in the presence of B in the unimolecular reaction=k₁/k₂.

Fersht cites examples in which the effective local concentration defined in this way is enormous (e.g., >10⁵ M). The enormous effective local concentrations leads to the insight that a hapten bearing a weakly reactive electrophile can diffuse intact through a dilute solution of nucleophiles, and still bind to the antibody antigen recognition domain and undergo attack by a nucleophilic sidechain of the antibody.

In addition to the antibodies and antibody-chelate pairs of the invention, in a fourth aspect, there is also provided a method of using the compositions of the invention to treat a patient for a disease or condition or to diagnose the disease or condition. The method comprises the steps of: (a) administering to the patient a mutant antibody comprising; (i) a antigen recognition domain that specifically binds to the metal chelate; (ii) a reactive site not present in the wild-type of the antibody and, wherein the reactive site is in a position proximate to or within the antigen recognition domain; and (iii) a targeting moiety that binds specifically to a cell thereby forming a complex between the mutant antibody and the cell. The binding of the antibody to the cell can be mediated by any cell surface structure, for example, cell surface receptors and cell surface antigens. Following step (a), the metal chelate is administered to the patient. The metal chelate comprises a pendant reactive functional group having a reactivity complementary to the reactivity of the reactive site of the antibody. Thus, the chelate and the antibody bind to form an antibody-antigen (chelate) pair, the reactive groups of which subsequently react to form a covalent bond between the antibody and the antigen.

In addition to the method described above, the present invention also provides a method in which the tissue is pretargeted with the antibodies described herein. In this aspect, the method comprises the steps of: (a) administering to a patient a mutant antibody of the invention. The mutant antibody comprises: (i) a antigen recognition domain that specifically binds to the metal chelate; (ii) a reactive site not present in the wild-type of the antibody (the reactive site is in a position proximate to or within the antigen recognition domain). In some embodiments, the mutant antibody comprise a targeting moiety that binds specifically to a component or structure on the surface of a cell, thereby forming a complex between the cell and the mutant antibody. Following step (a), a metal chelate is administered to the patient. The chelate specifically binds to the antibody forming an antibody-antigen complex. Moreover, the chelate comprises a reactive functional group having a reactivity complementary to that of the antibody reactive site. After the antibody-antigen complex is formed, the reactive site of the antibody and that of the metal chelate react to form a covalent bond between the mutant antibody and the metal chelate.

The compositions and methods of the present invention are described in greater detail below.

### BRIEF DESCRIPTION OF THE DRAWINGS

FIG. 1 shows the sequences for the V_{L} chain of 2D12.5 (SEQ ID NO: 1) and the sequences for CDR1, CDR2, and CDR3 for the V_{L} chain of2D12.5 (SEQ ID NOS: 2, 3, and 4, respectively). FIG. 1 also shows the sequences for the V_{H} chain (SEQ ID NO: 5) and the sequences for CDR1, CDR2, and CDR3 for the V_{H} chain of 2D12.5 (SEQ ID NOS: 6, 7, and 8, respectively).

FIG. 2 shows the alignment of the amino acid sequence of the V_{H} chain of2D12.5. In particular, FIG. 2 shows the alignment of the native hybridoma sequence, the native cloned hybridoma sequence, the N87D sequence, the N87D_G53C sequence, the N87D_G54C sequence, and the N87D_G55C sequence (SEQ ID NOS.: 9, 10, 11, 12, 13, and 14, respectively). Note that the native hybridoma sequence shown corresponds to amino acids 2-119 of the V_{H} chain of 2D12.5 as set forth in SEQ ID NO:5. Therefore, N87D is N88D, G53C is G54C, G54C is G55C, and G55C is G56C if the Kabat standard numbering system is used to determine the positions of amino acid residues in an antibody heavy chain or light chain (see, e.g., Kabat et al., Sequences of Proteins of Immunological Interest 5^{th} Ed., NIH Publication No. 91-3242 (1991)).

FIG. 3 shows the alignment of the nucleotide sequence of the V_{H} chain of 2D12.5. In particular, FIG. 3 shows the alignment of the native hybridoma sequence, the native cloned hybridoma sequence, the N87D sequence, the N87D_G53C sequence, the N87D_G54C sequence, and the N87D_G55C sequence (SEQ ID NOS.: 15, 16, 17, 18, 19, and 20, respectively).

FIG. 4 shows the alignment of the amino acid sequence of the V_{L} chain of 2D12.5. In particular, FIG. 4 shows the alignment of the native hybridoma sequence, the native cloned hybridoma sequence, and the N53C sequence (SEQ ID NOS.: 21, 22, and 23,respectively). Note that the native hybridoma sequence shown corresponds to amino acids 2-110 of the V_{L} chain of 2D12.5 as set forth in SEQ ID NO:1. Therefore, N53C is N54C, if the Kabat standard numbering system is used.

FIG. 5 shows the alignment of the nucleotide sequence of the V_{L} chain of 2D12.5. In particular, FIG. 5 shows the alignment of the native hybridoma sequence, the native cloned hybridoma sequence, and the N53C sequence (SEQ ID NOS.: 24, 25, and 26, respectively)

FIG. 6 shows the alignment of the amino acid sequence of the chimeric V_{L} chain of 2D 12.5 fused to the C_{L} kappa chain of a human anti-tetanus toxoid antibody. In particular, FIG. 6 shows the alignment of the native cloned hybridoma sequence, the N53C sequence fused to the C_{L} kappa chain, the native hybridoma sequence fused to the C_{L} kappa chain, and the C_{L} kappa chain of the human anti-tetanus toxoid antibody template for gene assembly (SEQ ID NOS.: 27, 28, 29 and 30, respectively).

FIG. 7 shows the alignment of the nucleotide sequence of the chimeric V_{L} chain of 2D12.5 fused to the C_{L} kappa chain of a human anti-tetanus toxoid antibody. In particular, FIG. 7 shows the alignment of the native cloned hybridoma sequence, the N53C sequence fused to the C_{L} kappa chain, the native hybridoma sequence fused to the C_{L} kappa chain, and the C_{L} kappa chain of the human anti-tetanus toxoid antibody template for gene assembly (SEQ ID NOS.: 31, 32, 33, and 34, respectively).

FIG. 8 shows the alignment of the amino acid sequence of the chimeric V_{H} chain of 2D12.5 fused to the CH1 chain of a human anti-tetanus toxoid antibody. In particular, FIG. 8 shows the alignment of the native cloned, hybridoma sequence fused to the CH1 chain, the N87D sequence fused to the CH1 chain, the N87D_G53C sequence fused to the CH1 chain, the N87D_G54C sequence fused to the CH1 chain, and the N87D_G55C sequence fused to the CH1 chain, the CH1 chain expected sequence, and the native hybridoma sequence fused to the CH1 chain, (SEQ ID NOS.: 35, 36, 37, 38, 39, 40, and 41, respectively).

FIG. 9 shows the alignment of the nucleotide sequence of the chimeric V_{H} chain of 2D12.5 fused to the CH1 chain of a human anti-tetanus toxoid antibody. In particular, FIG. 9 shows the alignment of the native cloned, hybridoma sequence fused to the CH1 chain, the N87D sequence fused to the CH1 chain, the N87D_G53C sequence fused to the CH1 chain, the N87D_G54C sequence fused to the CH1 chain, and the N87D_G55C sequence fused to the CH1 chain, the CH1 chain expected sequence, and the native hybridoma sequence fused to the CH1 chain, (SEQ ID NOS.: 42, 43, 44, 45, 46, 47, and 48, respectively).

FIG. 10 is a diagram depicting the strategy for assembly of the chimeric V_{H} chain of 2D12.5 fused to the CH1 chain of a human anti-tetanus toxoid antibody.

FIG. 11 is a diagram depicting the strategy for assembly of the chimeric V_{L} chain of 2D 12.5 fused to the C_{L} kappa chain of a human anti-tetanus toxoid antibody.

FIG. 12 is a graphical display showing binding of stably transfected Drosophila S2 cells expressing the chimeric 2D12.5 Fab gene products (native and site-directed cysteine mutants) to Y-DOTA. Binding curves were determined from non-competitive ELISA assays incorporating dilutions of media containing expressed gene products. The relative amount of expressed chimeric Fab were measured using anti-V5 epitope-HRP conjugate and a visible TMB (3,3′,5,5′-tetramethyl benzidine) substrate.

FIG. 13 is graphical display showing the relative binding of metal-DOTA complexes to antibody 2D 12.5. A representative set of competitive binding curves obtained from ELISA experiments described in Example 3 below. Error bars (representing the standard error of the mean) are shown, but are generally smaller than the data points.

FIG. 14 is a graphical display showing the dependence of the standard Gibbs Free Energy of binding on rare earth ionic radius shows thermodynamically elastic binding behavior between antibody 2D12.5 and rare earth-DOTA complexes. Elements plotted in the order Sc (open circle), Lu, Yb, Tm, Er, Ho, Y (open diamond), Dy, Tb, Gd, Eu, Sm, Nd, Pr, Ce, La. ΔΔG values relative to Y-DOTA, set at 0. Error bars represent standard error of the mean.

FIG. 15 is a graphical display showing that time-resolved 280 nm excitation yields a marked, linear enhancement in the green 545 nm emission of the Tb-DOTA bound to 2D12.5. The enhancement is not observed for Tb-DOTA and a non-binding IgG control.

FIG. 16 is a graphical display showing the relative binding curves of 2D12.5 for Y-DOTA isomers and Y-DTPA. 2D12.5 binds both the (R)— and (S)-isomer of 2-(4-nitrobenzyl)-DOTA when the coordinated metal is Y³⁺ (the same behavior is expected for the other rare earths). The (S)-isomer confers Λ-helicity, while the (R)-isomer confers Δ-helicity to the acetate arms. The Λ-helicity is observed in the crystal structure for 2D12.5 and is the preferred isomer for binding. However, the antibody tolerartes the (R)-isomer with Δ-helicity, and the affinity decreases less than an order of magnitude as compared to the (S), Λ isomer. Y-DOTA (no-sidearm) exists in solution as a racemic mixture of the coordination isomers. As expected, the binding affinity for racemic Y-DOTA is between that observed for the (S)— and (R)-isomers of 2-(4-nitrobenzyl)-DOTA.

FIG. 17 is a graphical display showing relative binding curves of Y-DOTA molecules with different sidechain locations. Changing the location of the sidechain of DOTA causes a decrease in the binding affinity, but the affinity of the (5-Amino-2-methoxy-phenyl)-carboxymethyl)-DOTA is still sufficiently strong to consider for further applications. Evaluatoin of the crystal structure seems to indicate that shorter substitutions at the position may bind with higher affinity. Substiutions at other locations may yield reasonably high affinities as well. The (5-Amino-2-methoxy-phenyl)-carboxymethyl)-DOTA analyzed in this experiment was racemic, so it is not clear which isomer binds with higher affinity. The low pKa of the carboxymethyl proton makes it difficult to prepare a chirally pure molecule. Substitution as observed in the (S)-2-(4-nitrobenzyl)-DOTA is clearly stronger.

FIG. 18 is a graphical display showing the relative binding affinities of the NBD complexes of various metal ions relative to Y-NBD.

FIG. 19 shows the crystal structure of 2D12.5 bound to metal complexes. There are two possible orientations for the chelate in the binding pocket. The G54C, G55C and G56C (heavy chain) mutants are designed to bind permanently when the chelate is in one binding mode while the N53C (light chain) mutant is designed to bind permanently when the chelate is in the other binding mode.

FIG. 20 shows theoretical models of the various 2D12.5 single-cysteine mutant binding pockets bound to Y-AABD in the first binding mode. The N53C light chain mutant appears unfavorable for forming a permanent bond with the ligand when the ligand is in this particular binding mode.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition, i.e., an antigen recognition domain. As used herein, “antigen recognition domain” means that part of the antibody, recombinant molecule, the fusion protein, or the immunoconjugate of the invention which recognizes the target or portions thereof. Typically the antigen recognition domain comprises the variable region of the antibody or a portion thereof, e.g., one, two, three, four, five, six, or more hypervariable regions. The terms “V_{H}” or “VH” refer to the variable region of an immunoglobulin heavy chain, including an Fv, scFv, dsFv or Fab. The terms “V_{L}” or “VL” refer to the variable region of an immunoglobulin light chain, including an Fv, scFv , dsFv or Fab.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). Thus, the term antibody, as used herein, also includes antibody fragments either produced by the modification of whole antibodies.

As used herein, “fragment” is defined as at least a portion of the variable region of the immunoglobulin molecule, which binds to its target, i.e. complementarity determining regions (CDRs), V_{L} (light chain variable region), V_{H} (heavy chain variable region), Fab, F(ab)2′ and any combination of those or any other portion of an immunoglobulin peptide capable of binding to target antigen (see, e.g., Fundamental Immunology (Paul ed., 4th. 1999). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term antibody also includes bivalent or bispecific molecules, diabodies, triabodies, and tetrabodies. Bivalent and bispecific molecules are described in, e.g., Kostelny et al., J. Immunol. 148: 1547 (1992), Pack and Pluckthun, Biochemistry 31: 1579 (1992), Zhu et al. Protein Sci. 6: 781 (1997), Hu et al. Cancer Res. 56: 3055 (1996), Adams et al., Cancer Res. 53: 4026 (1993), and McCartney, et al., Protein Eng. 8: 301 (1995).

A “humanized antibody” refers to an antibody in which the antigen binding loops, i.e., complementarity determining regions (CDRs), comprised by the V_{H} and V_{L} regions are grafted to a human framework sequence. Typically, the humanized antibodies have the same binding specificity as the non-humanized antibodies described herein. Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al., Nature 321: 522 (1986); and Verhoyen et al., Science 239: 1534 (1988). Humanized antibodies are further described in, e.g., Winter and Milstein, Nature 349: 293 (1991).

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively,. percent identity can be any integer from 25 to 100. More preferred embodiments include at least: 25%, 30%, 35%, 40%, 45%, 50%, 55%, 60%, 65%, 70%, 75%, 80%, 85%, 90%, 95%, or 99% or higher, compared to a reference sequence using the programs described herein, preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. “Substantial identity” of amino acid sequences for these purposes normally means that a polypeptide comprises a sequence that has at least 40% sequence identity to the reference sequence. Preferred percent identity of polypeptides can be any integer from 40 to 100. More preferred embodiments include at least 60%, 65%, 70%, 75%, 80%, 85%, 90%, 95%, or 99%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Optimal alignment of sequences for comparison may be conducted by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. U.S.A. 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

A preferred example of algorithms that are suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignmnent. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both

Another indication that nucleotide sequences are substantially identical is if two molecules hybridize to each other, or to a third nucleic acid, under moderately, and preferably 25 highly, stringent conditions. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

For the purpose of the invention, suitable “moderately stringent conditions” include, for example, prewashing in a solution of 5×SSC, 0.5% SDS, 1.0 mM EDTA (pH 8.0), hybridizing at 50° C.-65° C., 5×SSC overnight, followed by washing twice at 65° C. for 20 minutes with each of 2×, 0.5× and 0.2×SSC (containing 0.1% SDS). Such hybridizing DNA sequences are also within the scope of this invention. As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore or another moiety.

“Peptide,” “polypeptide” or “protein” refers to a polymer in which the monomers are amino acids and are joined together through amide bonds, alternatively referred to as a polypeptide. When the amino acids are a-amino acids, either the L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups alos include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂,—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-⁴-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The symbol [symbol] , whether utilized as a bond or displayed perpendicular to a bond indicates the point at which the displayed moiety is attached to the remainder of the molecule, solid support, etc.

“Non-covalent protein binding groups” are moieties that interact with an intact or denatured polypeptide in an associative manner. The interaction may be either reversible or 25 irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, 30 thiophosphate, phosphonate, carboxylate, boronate, sulfate, sulfone, sulfonate, thiosulfate, and thiosulfonate.

The term “targeting moiety” is intended to mean a moiety that is (1) able to direct the entity to which it is attached (e.g., therapeutic agent or marker) to a target cell, for example to a specific type of tumor cell or (2) is preferentially activated at a target tissue, for example a tumor. The targeting group can be a small molecule, which is intended to include both non-peptides and peptides. The targeting group can also be a macromolecule, which includes saccharides, lectins, receptors, ligand for receptors, proteins such as BSA, antibodies, and so forth.

As used herein, an “immunoconjugate” means any molecule or ligand such as an antibody or growth factor (i.e., hormone) chemically or biologically linked to a cytotoxin, a radioactive agent, an anti-tumor drug or a therapeutic agent. The antibody or growth factor may be linked to the cytotoxin, radioactive agent, anti-tumor drug or therapeutic agent at any location along the molecule so long as the antibody is able to bind its target. Examples of immunoconjugates include immunotoxins and antibody conjugates.

As used herein, “selectively killing” means killing those cells to which the antibody binds.

As used herein, examples of “carcinomas” include bladder, breast, colon, larynx, liver, lung, ovarian, pancreatic, rectal, skin, spleen, stomach, testicular, thyroid, and vulval carcinomas.

As used herein, an “effective amount” is an amount of the antibody, immunoconjugate, which selectively kills cells or selectively inhibits the proliferation thereof.

As used herein, “therapeutic agent” means any agent useful for therapy including anti-tumor drugs, cytotoxins, cytotoxin agents, and radioactive agents.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), interferons and radioactive agents.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, “a radioactive agent” includes any radioisotope, which is effective in destroying a tumor. Examples include, but are not limited to, indium-111,Y-90, Lu-177, Sm-153, Er-169, Dy-165, Cu-67, cobalt-60 and X-rays. Additionally, naturally occurring radioactive elements such as uranium, radium, and thorium, which typically represent mixtures of radioisotopes, are suitable examples of a radioactive agent.

As used herein, “administering” means oral administration, administration as a suppository, topical contact, intravenous, intraperitoneal, intramuscular, intralesional or subcutaneous administration, or the implantation of a slow-release device e.g., a miniosmotic pump, to the subject.

As used herein, “cell surface antigens” means any cell surface antigen which is generally associated with cells involved in a pathology (e.g., tumor cells), i.e., occurring to a greater extent as compared with normal cells. Such antigens may be tumor specific. Alternatively, such antigens may be found on the cell surface of both tumorigenic and non-tumorigenic cells. These antigens need not be tumor specific. However, they are generally more frequently associated with tumor cells than they are associated with normal cells.

As used herein, “tumor targeted antibody” means any antibody, which recognizes cell surface antigens on tumor (i.e., cancer) cells. Although such antibodies need not be tumor specific, they are tumor selective, i.e. bind tumor cells more so than it does normal cells.

As used herein, “pharmaceutically acceptable carrier” includes any material which when combined with the antibody retains the antibody's immunogenicity and non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

### DETAILED DESCRIPTION OF THE INVENTION AND THE PREFERRED EMBODIMENTS

The present invention provides compositions for delivering therapeutic and diagnostic agents directly to cells involved in a disease or other pathology. The compositions of the invention include reactive therapeutic or diagnostic species and reactive antibodies that specifically bind the therapeutic or diagnostic species and, subsequent to the specific binding event, form a covalent bond via the reactive site of the antibody and the pendant reactive functional group of the therapeutic or diagnostic species. Also provided are methods of treating a patient using the compounds described herein.

The present invention is illustrated by reference to the use of reactive metal chelates as an exemplary embodiment. The use of metal chelates to illustrate the concept of the invention is not intended to define or limit the scope of the invention. Those of skill in the art will readily appreciate that the concepts underlying the compositions and methods described herein are equally applicable to any therapeutic or diagnostic agent to which an antibody can be raised (e.g., antitumor drugs, cytotoxins, etc.).

A. The Compositions

In a first aspect, the present invention provides a mutant antibody comprising a reactive site that is not present in the wild-type of the antibody. The antibody also has an antigenrecognition domain that specifically binds to a metal chelate against which the wild-type antibody is raised. The reactive site of the mutant antibody is in a position proximate to or within the antigen recognition domain, such that the chelate and the antibody are able to form a covalent bond.

1. The Antibodies

The present invention provides reactive mutant antibodies that specifically bind to reactive metal chelates. For preparation of monoclonal or polyclonal antibodies, any technique known in the art can be used (see, e.g., Kohler & Milstein, Nature 256: 495-497 (1975); Kozbor et al., Immunology Today 4: 72 (1983); Cole et al., pp. 77-96 in MONOCLONAL ANTIBODIES AND CANCER THERAPY, Alan R. Liss, Inc. (1985)). 100911 Methods of producing of polyclonal antibodies are known to those of skill in the art. In an exemplary method, an inbred strain of mice (e.g., BALB/C mice) or rabbits is immunized with the chelate or a close structural analogue using a standard adjuvant, such as Freund's adjuvant, and a standard immunization protocol. Alternatively, or in addition to the use of an adjuvant, the chelate is coupled to a carrier that is itself immunogenic (e.g., keyhole limpit hemocyanin (“KLH”). The animal's immune response to the immunogen preparation is monitored by taking test bleeds and determining the titer of reactivity to the immunogen, i.e., chelate. When appropriately high titers of antibody to the immunogen are obtained, blood is collected from the animal and antisera are prepared. Further fractionation of the antisera to enrich for antibodies reactive to the protein can be done if desired.

Monoclonal antibodies are obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Monoclonal antibodies and polyclonal sera are collected and titered against the immunogen in an immunoassay, for example, a solid phase immunoassay with the immunogen immobilized on a solid support. Typically, polyclonal antisera with a titer of 10⁴ or greater are selected and tested for cross reactivity against different chelates, using a competitive binding immunoassay. Specific polyclonal antisera and monoclonal antibodies will usually bind with a K_{d} of at least about 0.1 mM, more usually at least about 1 μM, preferably, at least about 0.1 μM or better, and most preferably, 0.01 μM or better.

Techniques for the production of single chain antibodies (U.S. Pat. No. 4,946,778) can be adapted to produce antibodies to reactive chelates and other diagnostic, analytical and therapeutic agents. Also, transgenic mice, or other organisms such as other mammals, may be used to express humanized antibodies. Alternatively, phage display technology can be used to produce and identify antibodies and heteromeric Fab fragments that specifically bind to selected antigens (see, e.g., McCafferty et al., Nature 348: 552-554 (1990); Marks et al., Biotechnology 10: 779-783 (1992)).

In an exemplary embodiment, an animal, such as a rabbit or mouse is immunized with a chelate, or an immunogenic construct. The antibodies produced as a result of the immunization are preferably isolated using standard methods.

In a still further preferred embodiment, the antibody is a humanized antibody. “Humanized” refers to a non-human polypeptide sequence that has been modified to minimize immunoreactivity in humans, typically by altering the amino acid sequence to mimic existing human sequences, without substantially altering the function of the polypeptide sequence (see, e.g., Jones et al., Nature 321: 522-525 (1986), and published UK patent application No. 8707252).

In another preferred embodiment, the present invention provides an antibody, as described above, further comprising a member selected from detectable labels, biologically active agents and combinations thereof attached to the antibody.

When the antibody is conjugated to a detectable label, the label is preferably a member selected from the group consisting of radioactive isotopes, fluorescent agents, fluorescent agent precursors, chromophores, enzymes and combinations thereof. Methods for conjugating various groups to antibodies are well known in the art. For example, a detectable label that is frequently conjugated to an antibody is an enzyme, such as horseradish peroxidase, alkaline phosphatase, β-galactosidase, and glucose oxidase.

In an exemplary embodiment of the present invention, horseradish peroxidase is conjugated to an antibody raised against a reactive chelate. In this embodiment, the saccharide portion of the horseradish peroxidase is oxidized by periodate and subsequently coupled to the desired immunoglobin via reductive amination of the oxidized saccharide hydroxyl groups with available amine groups on the immunoglobin.

Methods of producing antibodies labeled with small molecules, for example, fluorescent agents, are well known in the art. Fluorescent labeled antibodies can be used in immunohistochemical staining (Osborn et al., Methods Cell Biol. 24: 97-132 (1990); in flow cytometry or cell sorting techniques (Ormerod, M. G. (ed.), FLOW CYTOMETRY. A PRACTICAL APPROACH, IRL Press, New York, 1990); for tracking and localization of antigens, and in various double-staining methods (Kawamura, A., Jr., FLUORESCENT ANTIBODY TECHNIQUES AND THEIR APPLICATION, Univ. Tokyo Press, Baltimore, 1977).

Many reactive fluorescent labels are available commercially (e.g., Molecular Probes, Eugene, Oreg.) or they can be synthesized using art-recognized techniques. In an exemplary embodiment, an antibody of the invention is labeled with an amine-reactive fluorescent agent, such as fluorescein isothiocyanate under mildly basic conditions. For other examples of antibody labeling techniques, see, Goding, J. Immunol. Methods 13: 215-226 (1976); and in, MONOCLONAL ANTIBODIES: PRINCIPLES AND PRACTICE, pp. 6-58, Academic Press, Orlando (1988).

Prior to constructing the mutagenized antibodies of the invention, it is often useful to prepare the wild-type anti-chelate antibody from an isolated nucleic acid encoding an antibody or a portion of an antibody of the invention. In a further preferred embodiment, the antibody fragment is an Fᵥ fragment. Fᵥ fragments of antibodies are heterodimers of antibody V_{H} (variable region of the heavy chain) and V_{L} domains (variable region of the light chain). They are the smallest antibody fragments that contain all structural information necessary for specific antigen binding. Fᵥ fragments are useful for diagnostic and therapeutic applications such as imaging of tumors or targeted cancer therapy. In particular, because of their small size, Fᵥ fragments are useful in applications that require good tissue or tumor penetration, because small molecules penetrate tissues much faster than large molecules (Yokota et al., Cancer Res., 52: 3402-3408 (1992)).

The heterodimers of heavy and light chain domains that occur in whole IgG, for example, are connected by a disulfide bond, but Fᵥ fragments lack this connection. Although native unstabilized Fᵥ heterodimers have been produced from unusual antibodies (Skerra et al., Science, 240: 1038-1041 (1988); Webber et al., Mol. Immunol. 4: 249-258 (1995), generally Fᵥ fragments by themselves are unstable because the V_{H} and V_{L} domains of the heterodimer can dissociate (Glockshuber et al., Biochemistry 29: 1362-1367 (1990)). This potential dissociation results in drastically reduced binding affinity and is often accompanied by aggregation.

Solutions to the stabilization problem have resulted from a combination of genetic engineering and recombinant protein expression techniques. Such techniques are of use in constructing the antibodies of the present invention. The most common method of stabilizing Fᵥs is the covalent connection of V_{H} and V_{L} by a flexible peptide linker, which results in single chain Fᵥ molecules (see, Bird et al., Science 242: 423-426 (1988); Huston et al., Proc. Natl. Acad. Sci. USA 16: 5879-5883 (1988)). The single chain Fᵥs (scFᵥs) are generally more stable than Fᵥs alone.

Another way to generate stable recombinant Fᵥs is to connect V_{H} and V_{L} by an interdomain disulfide bond instead of a linker peptide; this technique results in disulfide stabilized Fᵥ (dsFᵥ). The dsFᵥs solve many problems that can be associated with scFᵥs: they are very stable, often show full antigen binding activity, and sometimes have better affinity than scFᵥs (Reiter et al., Int. Cancer 58: 142-149 (1994)). Thus, in another preferred embodiment, the antibody of the invention is a dsFᵥs

Peptide linkers, such as those used in the expression of recombinant single chain antibodies, may be employed as the linkers and connectors of the invention. Peptide linkers and their use are well known in the art. (See, e.g., Huston et al., 1988; Bird et al., 1983; U.S. Pat. No. 4,946,778; U.S. Pat. No. 5,132,405; and Stemmer et al., Biotechniques 14:256-265 (1993)). The linkers and connectors are flexible and their sequence can vary. Preferably, the linkers and connectors are long enough to span the distance between the amino acids to be joined without putting strain on the structure. For example, the linker (gly₄ser)₃ is a useful linker because it is flexible and without a preferred structure (Freund et al., Biochemistry 33: 3296-3303 (1994)).

After the stabilized immunoglobin has been designed, a gene encoding at least F, or a fragment thereof is constructed. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)).

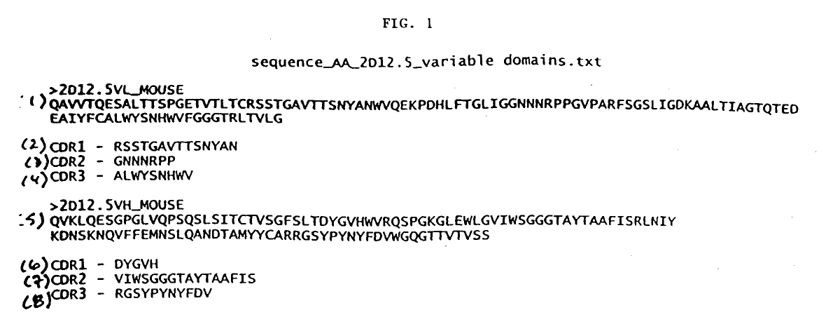

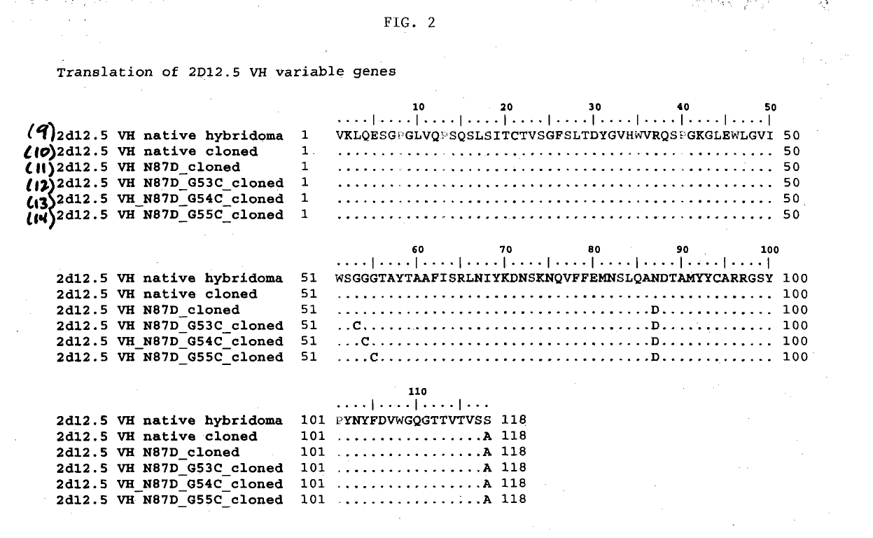

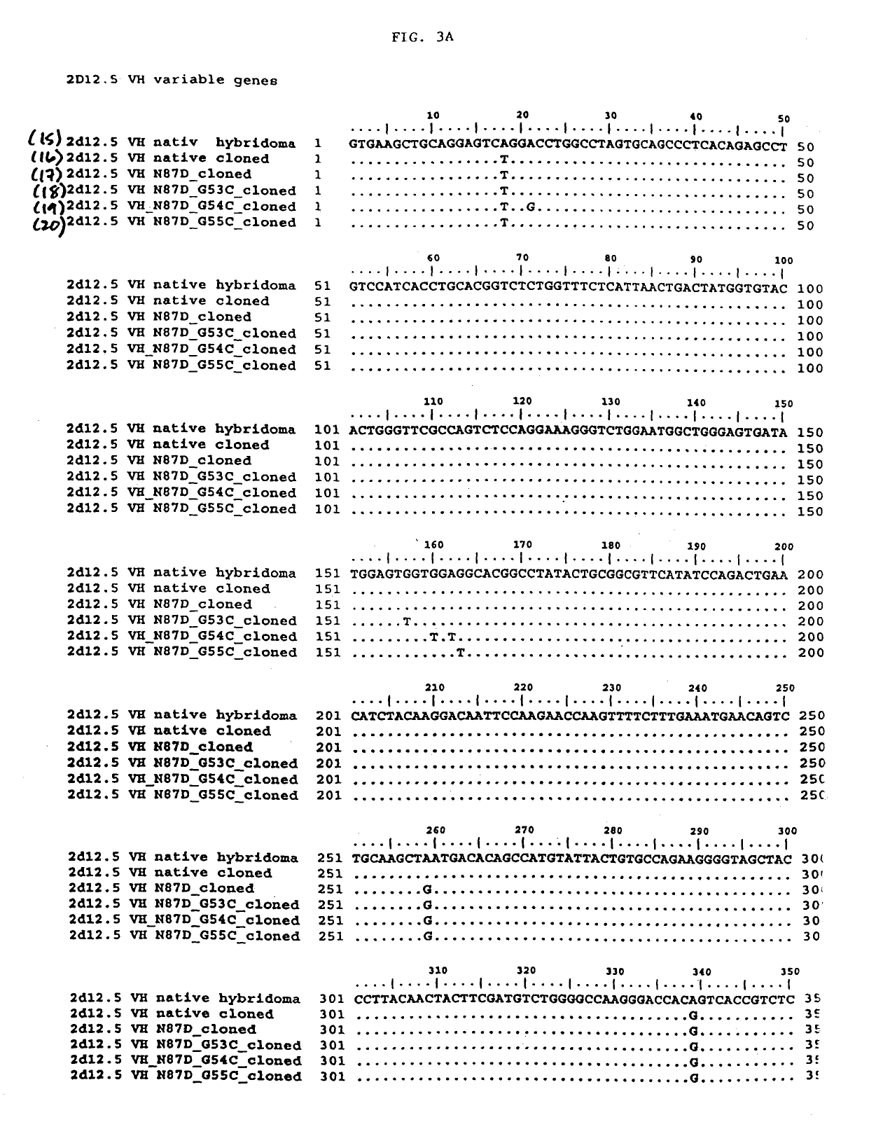

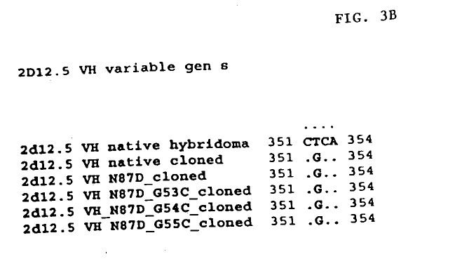

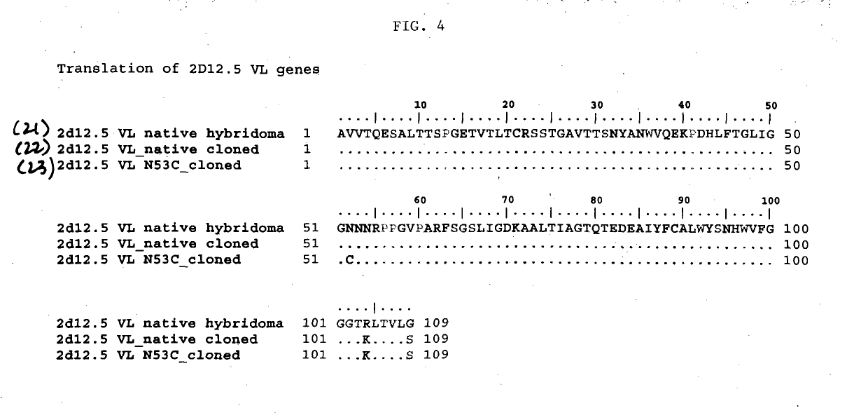

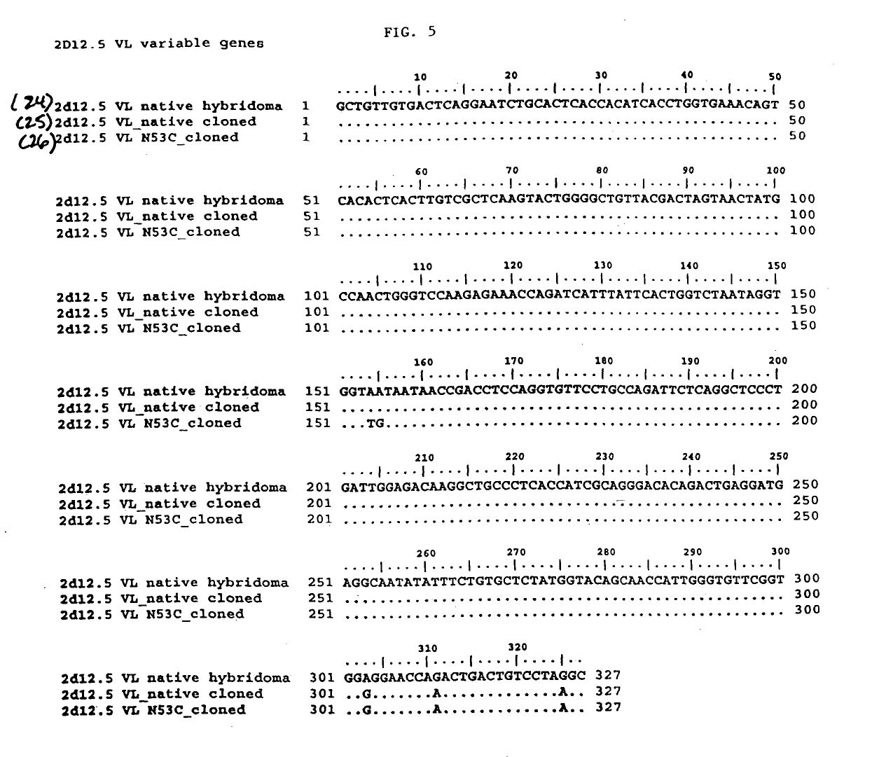

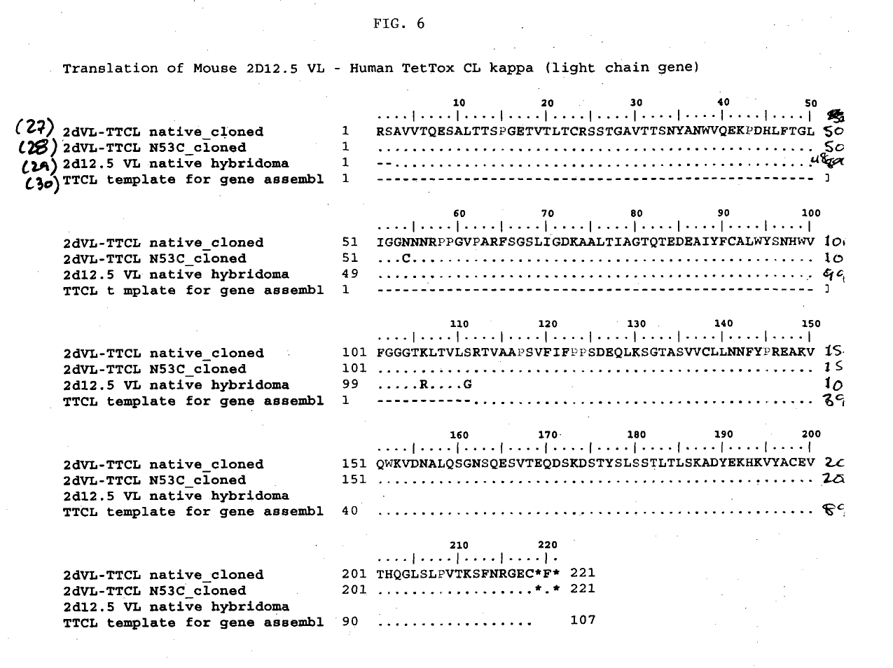

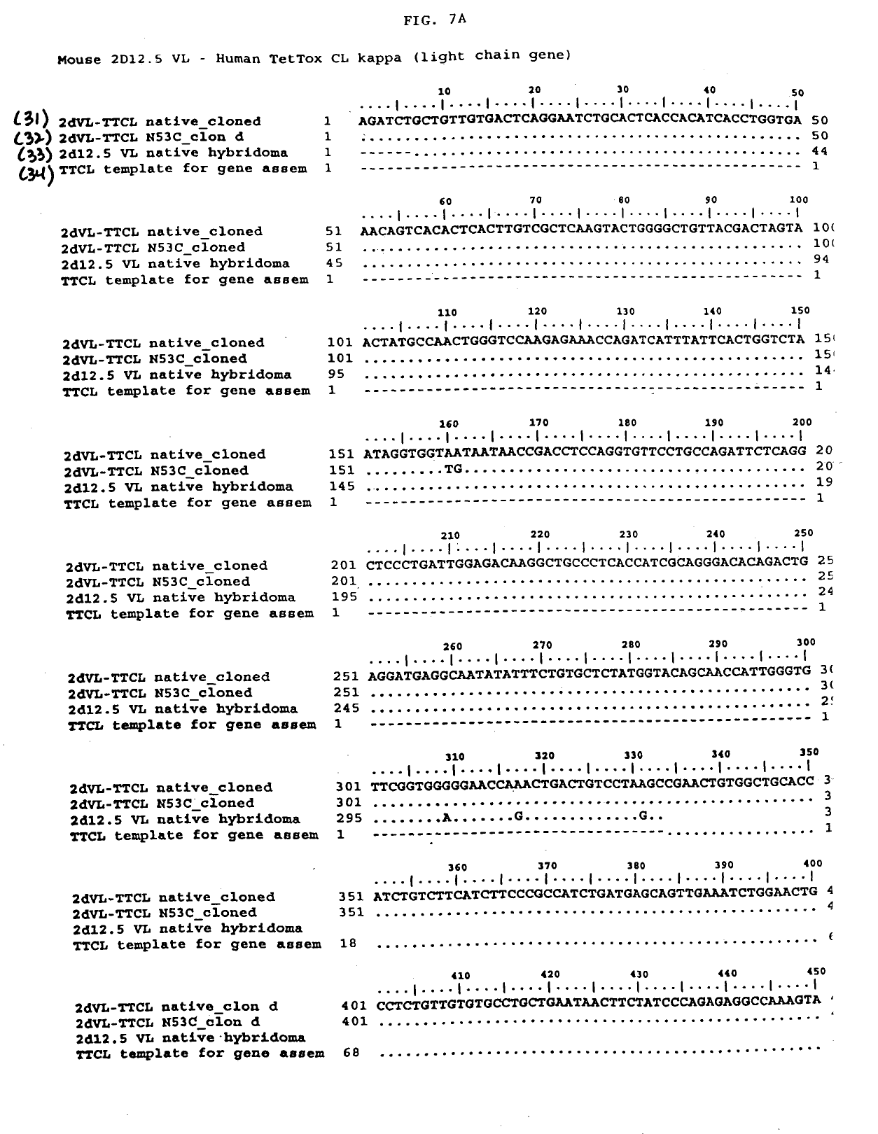

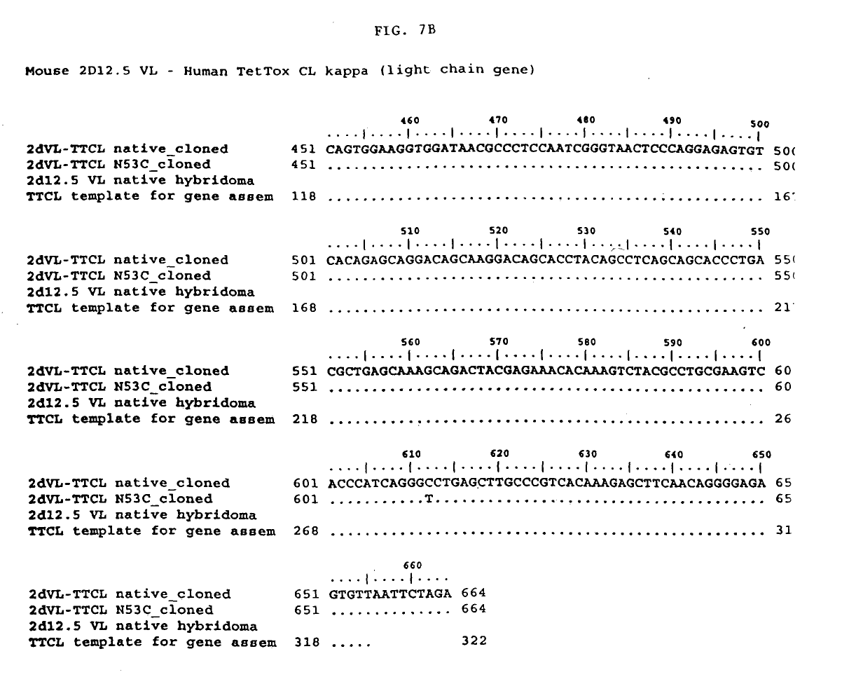

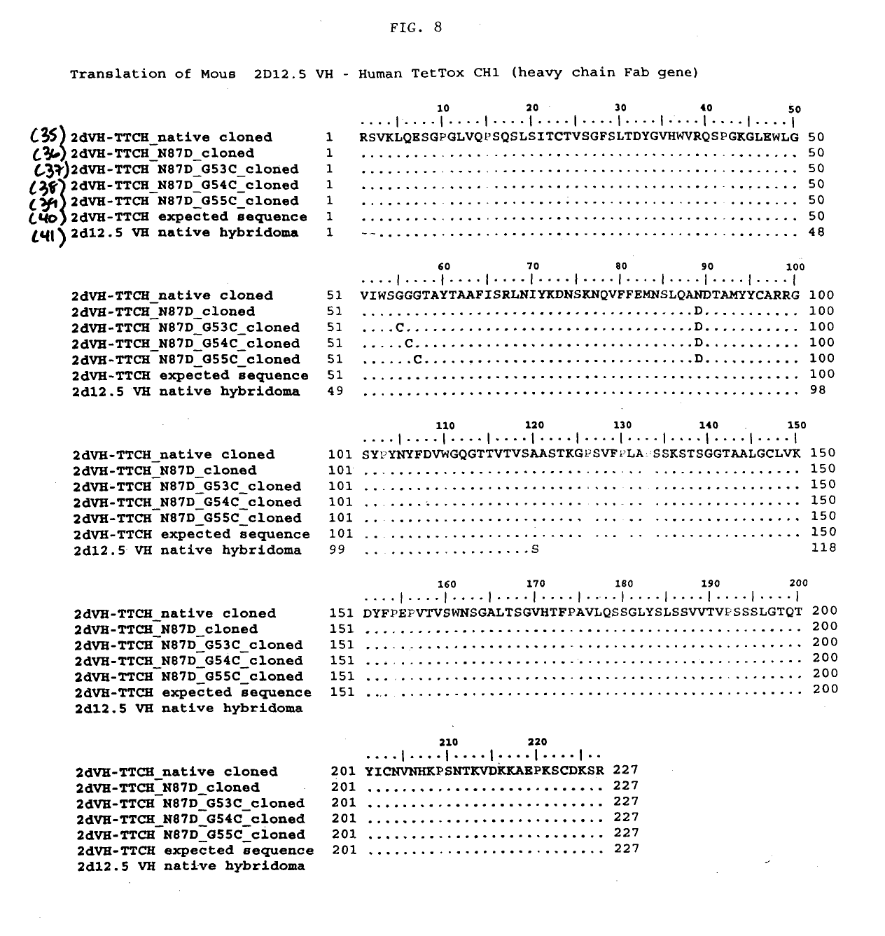

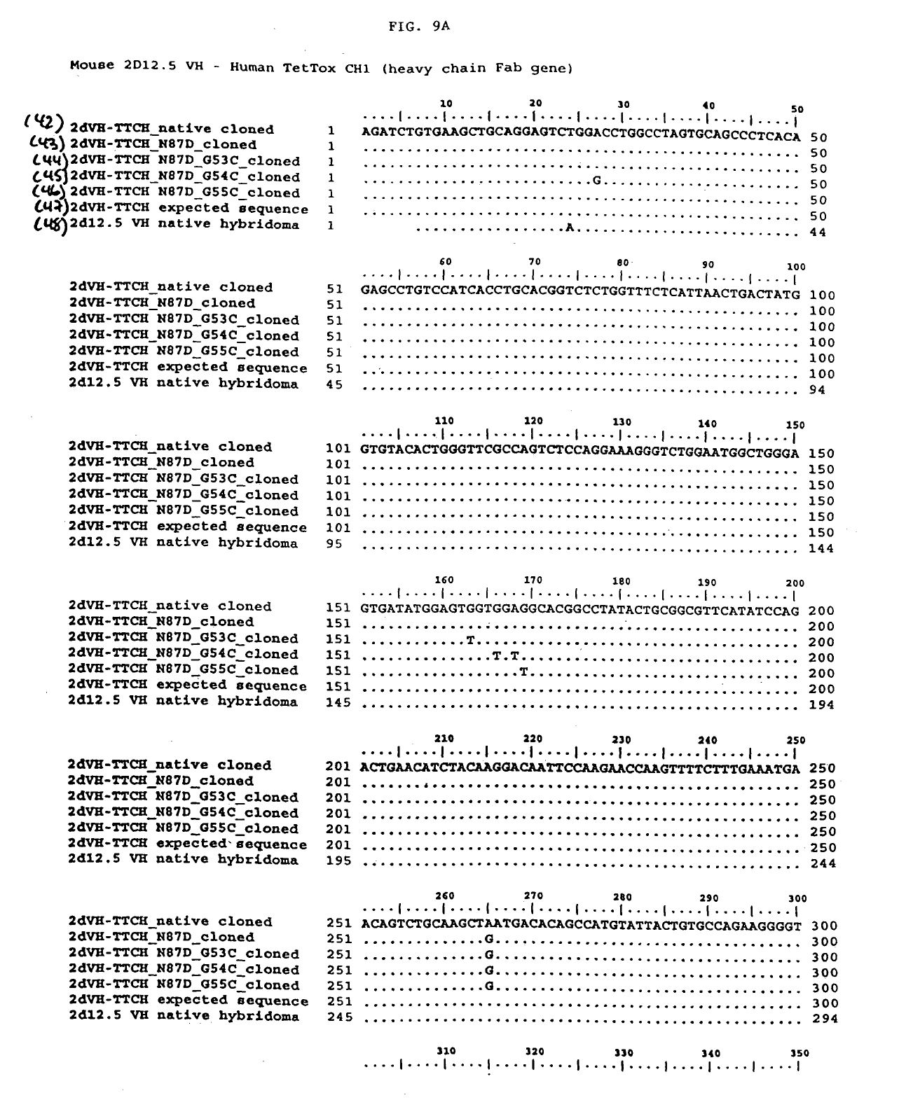

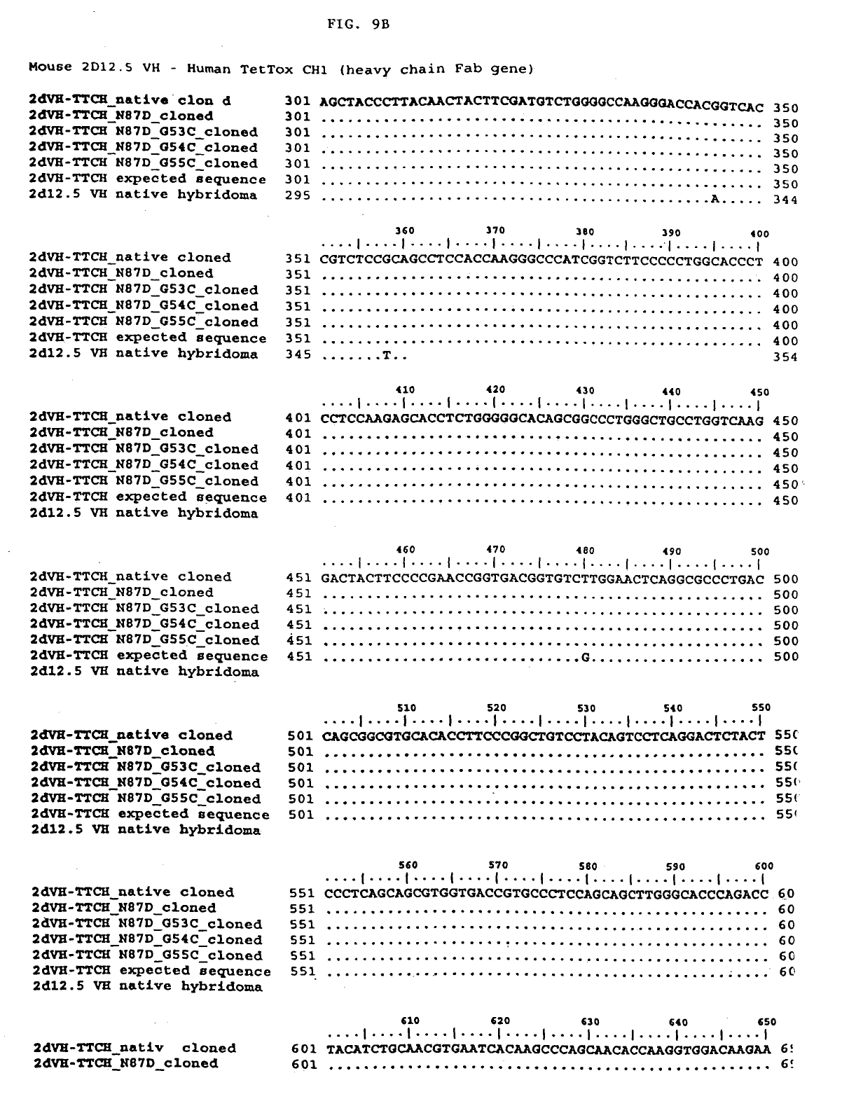

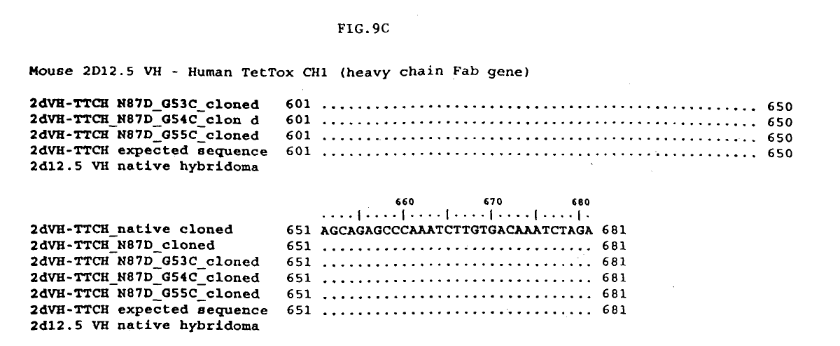

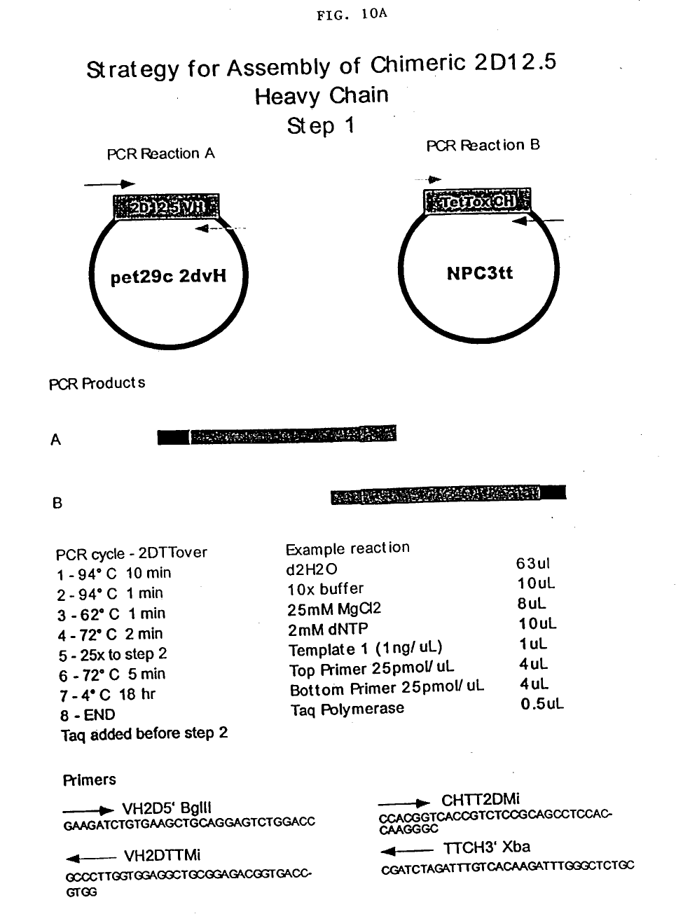

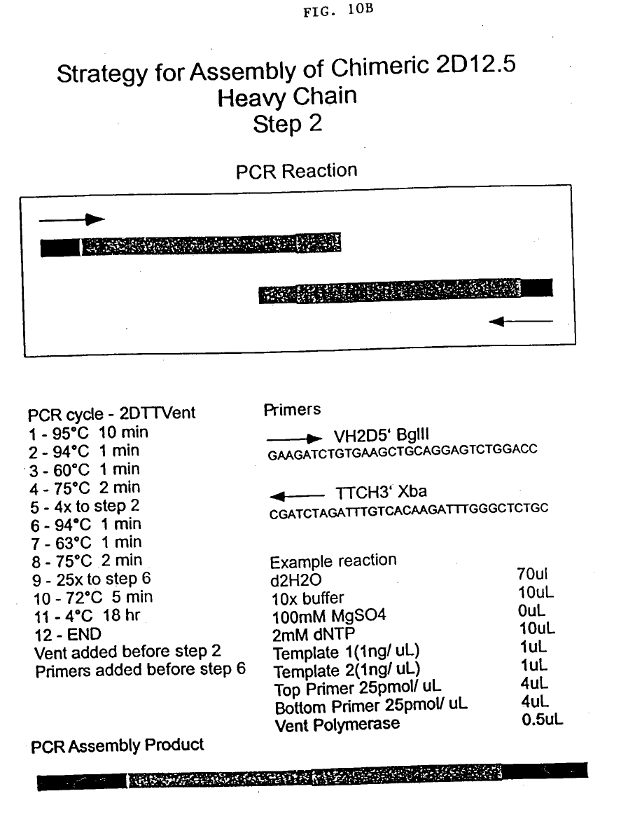

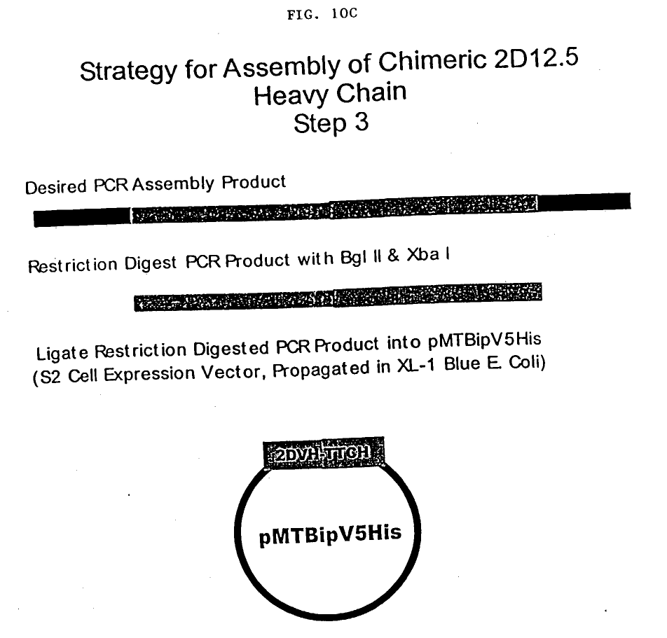

The present invention provides for the expression of nucleic acids corresponding to the wild-type of essentially any antibody that can be raised against a metal chelate, and the modification of that antibody to include a reactive site. In a preferred embodiment, the Fab heavy chain of the wild-type antibody is the amino acid sequence set forth in SEQ ID NO.:5 (FIG. 1) or is encoded by the nucleic acid sequence set forth in SEQ ID NO.:16 (FIG. 3). In another preferred embodiment, the light-chain of the wild-type antibody is the amino acid sequence set forth in SEQ ID NO.:1 (FIG. 1) or is encoded by the nucleic acid sequence set forth in SEQ ID NO.:25 (FIG. 5). In yet another preferred embodiment, the invention provides a mutant of the light chain of 2D12.5 in which N-53 is substituted by C and that has the amino acid sequence set forth in SEQ ID NO.:23 (FIG. 4), or is encoded by the nucleic acid sequence set forth in SEQ ID NO: 26 (FIG. 5). In yet another preferred embodiment, the invention provides a mutant of the heavy-chain of 2D12.5 in which N-87 is replaced by D and that has the amino acid sequence set forth in SEQ ID NO.:11 (FIG. 2) or is encoded by the nucleic acid sequence set forth in SEQ ID NO: 17 (FIG. 3). In yet another preferred embodiment, the invention provides a mutant of the heavy-chain of 2D12.5 in which N-87 is replaced by D and G-53 is replaced by C, and that has the amino acid sequence set forth in SEQ ID NO.:12 (FIG. 2) or is encoded by the nucleic acid sequence set forth in SEQ ID NO: 18 (FIG. 3). In yet another preferred embodiment, the invention provides a mutant of the heavy-chain of 2D12.5 in which N-87 is replaced by D and G-54 is replaced by C, and that has the amino acid sequence set forth in SEQ ID NO.:13 (FIG. 2) or is encoded by the nucleic acid sequence set forth in SEQ ID NO: 19 (FIG. 3). In yet another preferred embodiment, the invention provides a mutant of the heavy-chain of 2D 12.5 in which N-87 is replaced by D and G-55 is replaced by C, and that has the amino acid sequence set forth in SEQ ID NO.:14 (FIG. 2) or is encoded by the nucleic acid sequence set forth in SEQ ID NO: 20 (FIG. 3).

Those of skill in the art will understand that substituting selected codons from the above-recited sequences with equivalent codons is within the scope of the invention. Oligonucleotides that are not commercially available are preferably chemically synthesized according to the solid phase phosphoramidite triester method first described by Beaucage & Caruthers, Tetrahedron Letts. 22: 1859-1862 (1981), using an automated synthesizer, as described in Van Devanter et. al., Nucleic Acids Res. 12: 6159-6168 (1984). Purification of oligonucleotides is preferably by either native acrylamide gel electrophoresis or by anion-exchange HPLC as described in Pearson & Reanier, J. Chrom. 255: 137-149 (1983). The sequence of the cloned genes and synthetic oligonucleotides can be verified after cloning using art-recognized methods, e.g., the chain termination method for sequencing double-stranded templates of Wallace et al., Gene 16: 21-26 (1981).

One preferred method for obtaining specific nucleic acid sequences combines the use of synthetic oligonucleotide primers with polymerase extension or ligation on a mRNA or DNA template. Such a method, e.g., RT, PCR, or LCR, amplifies the desired nucleotide sequence, which is often known (see, U.S. Pat. Nos. 4,683,195 and 4,683,202). Restriction endonuclease sites can be incorporated into the primers. Amplified polynucleotides are purified and ligated into an appropriate vector. Alterations in the natural gene sequence can be introduced by techniques such as in vitro mutagenesis and PCR using primers that have been designed to incorporate appropriate mutations.

A particularly preferred method of constructing the immunoglobulin is by overlap extension PCR. In this technique, individual fragments are first generated by PCR using primers that are complementary to the immunoglobulin sequences of choice. These sequences are then joined in a specific order using a second set of primers that are complementary to “overlap” sequences in the first set of primers, thus linking the fragments in a specified order. Other suitable Fᵥ fragments can be identified by those skilled in the art. The immunoglobulin, e.g., Fᵥ, is inserted into an “expression vector,” “cloning vector,” or “vector.” Expression vectors can replicate autonomously, or they can replicate by being inserted into the genome of the host cell. Often, it is desirable for a vector to be usable in more than one host cell, e.g., in E. coli for cloning and construction, and in a mammalian cell for expression. Additional elements of the vector can include, for example, selectable markers, e.g., tetracycline resistance or hygromycin resistance, which permit detection and/or selection of those cells transformed with the desired polynucleotide sequences (see, e.g., U.S. Pat. No. 4,704,362). The particular vector used to transport the genetic information into the cell is also not particularly critical. Any suitable vector used for expression of recombinant proteins host cells can be used.

Expression vectors typically have an expression cassette that contains all the elements required for the expression of the polynucleotide of choice in a host cell. A typical expression cassette contains a promoter operably linked to the polynucleotide sequence of choice. The promoter used to direct expression of the nucleic acid depends on the particular application, for example, the promoter may be a prokaryotic or eukaryotic promoter depending on the host cell of choice. The promoter is preferably positioned about the same distance from the heterologous transcription start site as it is from the transcription start site in its natural setting. As is known in the art, however, some variation in this distance can be accommodated without loss of promoter function.

Promoters include any promoter suitable for driving the expression of a heterologous gene in a host cell, including those typically used in standard expression cassettes. In addition to the promoter, the recombinant protein gene will be operably linked to appropriate expression control sequences for each host. For E. coli this includes a promoter such as the T7, trp, tac, lac or lambda promoters, a ribosome binding site, and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The vectors can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes. One of skill in the art will appreciate that vectors comprising DNA encoding the V_{L} chain of an antibody and vectors comprising DNA encoding the V_{H} chain of an antibody can conveniently be separately transfected into different host cells. Alternately vectors comprising DNA encoding the V_{L} chain of an antibody and vectors comprising DNA encoding the V_{H} chain of an antibody may be cotransfected into the same host cells.

The wild-type antichelate-antibodies can be expressed in a variety of host cells, including E. coli, other bacterial hosts, yeast, and various higher eukaryotic cells such as the COS, CHO, and HeLa cells lines and myeloma cell lines. Methods for refolding single chain polypeptides expressed in bacteria such as E. coli have been described, are well-known and are applicable to the wild-type anti-chelate polypeptides. (See, e.g., Buchner et al., Analytical Biochemistry 205: 263-270 (1992); Pluckthun, Biotechnology 9: 545 (1991); Huse et al., Science 246: 1275 (1989) and Ward et al., Nature 341: 544 (1989)).

In a preferred embodiment, the present invention provides a polypeptide that is essentially homologous to the V_{L} sequence of 2D12.5, with the exception that serine-95 is replaced with a cysteine (FIG. 3).

Often, functional protein from E. coli or other bacteria is generated from inclusion bodies and requires the solubilization of the protein using strong denaturants, and subsequent refolding. In the solubilization step, a reducing agent must be present to dissolve disulfide bonds as is well-known in the art. Renaturation to an appropriate folded form is typically accomplished by dilution (e.g. 100-fold) of the denatured and reduced protein into refolding buffer.

Once expressed, the recombinant proteins can be purified according to standard procedures of the art, including ammonium sulfate precipitation, affinity columns, column chromatography, and the like (see, generally, Scopes, PROTEIN PURIFICATION (1982)). Substantially pure compositions of at least about 90 to 95% homogeneity are preferred, and those of 98 to 99% or more homogeneity are most preferred for pharmaceutical uses. Once purified, partially or to homogeneity as desired, the polypeptides may then be used therapeutically and diagnostically.

a. Bispecific Antibodies

In another preferred embodiment, the present invention provides for a reactive antibody that is bispecific for both a metal chelate and a targeting reagent or a target tissue, such as a tumor. Bispecific antibodies (BsAbs) are antibodies that have binding specificities for at least two different antigens. Bispecific antibodies can be derived from full length antibodies or antibody fragments (e.g. F(ab′)₂ bispecific antibodies). In a preferred embodiment, the bispecific antibody recognizes a reactive ¹¹¹In chelate of the invention and a human carcinoma cell.

Methods for making bispecific antibodies are known in the art. Traditional production of full-length bispecific antibodies is based on the co-expression of two immunoglobulin heavy chain-light chain pairs, where the two chains have different specificities (Millstein and Cuello, Nature 305: 537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., EMBO J. 10: 3655-3659 (1991)).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. One of skill in the art will appreciate that any immunoglobulin heavy chain known in the art may be fused to an antibody variable domain with the desired binding specificity. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies (see, for example, Suresh et al., Methods in Enzymology 121: 210 (1986)).

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al. (Science 229: 81 (1985)) describe a procedure wherein intact antibodies are proteolytically cleaved to generate F(ab′)₂ fragments. The fragments are reduced in the presence of the dithiol complexing agent sodium arsenite to stabilize vicinal dithiols and prevent intermnolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Recent progress has facilitated the direct recovery of Fab′-SH fragments from E. coli, which can be chemically coupled to form bispecific antibodies. Shalaby et al., J. Ex. Med., B 217-225 (1992) describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also, Rodrigues et al., Int. J. Cancers, (Suppl.) 7: 45-50 (1992).

Various techniques for making and isolating BsAb fragments directly from recombinant cell culture have also been described and are useful in practicing the present invention. For example, bispecific F(ab′)₂ heterodimers have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. (USA), 90: 6444-6448 (1993) has provided an alternative mechanism for making BsAb fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making BsAb fragments by the use of single-chain Fv (sFv) dimers has also been reported (see, Gruber et al., J. Immunol., 152: 5368 (1994)). Gruber et al., designed an antibody which comprised the V_{H} and V_{L} domains of a first antibody joined by a 25-amino-acid-residue linker to the V_{H} and V_{L} domains of a second antibody. The refolded molecule bound to fluorescein and the T-cell receptor and redirected the lysis of human tumor cells that had fluorescein covalently linked to their surface.

In addition to the preparation of wild-type antibodies that specifically bind to metal chelates, the present invention provides mutant antibodies that include a reactive site within their structure. The mutant antibodies are prepared by any method known in the art, most preferably by site directed mutagenesis of a nucleic acid encoding the wild-type antibody.

b. Site-Directed Mutaegnesis

The preparation of wild-type antibodies that bind to metal chelates is discussed above. The elements of the discussion above are also broadly applicable to aspects and embodiments of the invention in which site directed mutagenesis is used to produce mutant antibodies. The concept of site-directed mutagenesis as it applies to the present invention is discussed in greater detail to supplement, not to replace the discussion above.

The mutant antibodies are suitably prepared by introducing appropriate nucleotide changes into the DNA encoding the polypeptide of interest, or by in vitro synthesis of the desired mutant antibody. Such mutants include, for example, deletions from, or insertions or substitutions of, residues within the amino acid sequence of the polypeptide of interest so that it contains the proper epitope and is able to form a covalent bond with a reactive metal chelate. Any combination of deletion, insertion, and substitution is made to arrive at the final construct, provided that the final construct possesses the desired characteristics. The amino acid changes also may alter post-translational processes of the polypeptide of interest, such as changing the number or position of glycosylation sites. Moreover, like most mammalian genes, the antibody can be encoded by multi-exon genes.

For the design of amino acid sequence mutants of the antibodies, the location of the mutation site and the nature of the mutation will be determined by the specific polypeptide of interest being modified and the structure of the reactive chelate to which the antibody binds. The sites for mutation can be modified individually or in series, e.g., by: (1) substituting first with conservative amino acid choices and then with more radical selections depending upon the results achieved; (2) deleting the target residue; or (3) inserting residues of the same or a different class adjacent to the located site, or combinations of options 1-3.

A useful method for identification of certain residues or regions of the polypeptide of interest that are preferred locations for mutagenesis is called “alanine scanning mutagenesis,” as described by Cunningham and Wells, Science, 244: 1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the surrounding aqueous environment in or outside the cell. Those domains demonstrating functional sensitivity to the substitutions then are refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis is conducted at the target codon or region and the variants produced are screened for increased reactivity with a particular reactive chelate.

Amino acid sequence deletions generally range from about 1 to 30 residues, more preferably about 1 to 10 residues, and typically they are contiguous. Contiguous deletions ordinarily are made in even numbers of residues, but single or odd numbers of deletions are within the scope hereof. As an example, deletions may be introduced into regions of low homology among LFA-1 antibodies, which share the most sequence identity to the amino acid sequence of the polypeptide of interest to modify the half-life of the polypeptide. Deletions from the polypeptide of interest in areas of substantial homology with one of the binding sites of other ligands will be more likely to modify the biological activity of the polypeptide of interest more significantly. The number of consecutive deletions will be selected so as to preserve the tertiary structure of the polypeptide of interest in the affected domain, e.g., beta-pleated sheet or alpha helix.

Amino acid sequence insertions include amino- and/or carboxyl-terminal fusions ranging in length from one residue to polypeptides containing a hundred or more residues, as well as intra-sequence insertions of single or multiple amino acid residues. Intra-sequence insertions (i.e., insertions within the mature polypeptide sequence) may range generally from about 1 to 10 residues, more preferably 1 to 5, most preferably 1 to 3. Insertions are preferably made in even numbers of residues, but this is not required. Examples of insertions include insertions to the internal portion of the polypeptide of interest, as well as N— or C-terminal fusions with proteins or peptides containing the desired epitope that will result, upon fusion, in an increased reactivity with the chelate.

A third group of variants are amino acid substitution variants. These variants have at least one amino acid residue in the polypeptide molecule removed and a different residue inserted in its place. The sites of greatest interest for substitutional mutagenesis include one or two loops in antibodies. Other sites of interest are those in which particular residues of the polypeptide obtained from various species are identical among all animal species of the polypeptide of interest, this degree of conservation suggesting importance in achieving biological activity common to these molecules. These sites, especially those falling within a sequence of at least three other identically conserved sites, are substituted in a relatively conservative manner. Such conservative substitutions are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Substitution |
|---|---|
| Ala (A) | val; leu; ile |
| Arg (R) | lys; gln; asn |
| Asn (N) | gln; his; lys |
| Asp (D) | glu |
| Cys (C) | ser |
| Gln (Q) | asn |
| Glu (E) | asp |
| Gly (G) | pro; ala |
| His (H) | asn; gln; lys; arg |
| Ile (I) | leu; vat; met; ala |
| phe; | norleucine |
| Leu (L) | norleucine; ile; val; met; |
| | ala; phe |
| Lys (K) | arg; gln; asn |
| Met (M) | leu; phe; ile |
| Phe (F) | leu; val; ile; ala; leu |
| Pro (P) | ala |
| Ser (S) | thr |
| Thr (T) | ser |
| Trp (W) | tyr; phe |
| Tyr (Y) | trp; phe; thr; ser |
| Val (V) | ile; leu; met; phe; ala; |
| | norleucine |

In addition to the incorporation of the reactive site in the antibody structure, modifications in the function of the polypeptide of interest can be made by selecting substitutions that differ significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. Naturally occurring residues are divided -into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

It also may be desirable to inactivate one or more protease cleavage sites that are present in the molecule. These sites are identified by inspection of the encoded amino acid sequence, in the case of trypsin, e.g., for an arginyl or lysinyl residue. When protease cleavage sites are identified, they are rendered inactive to proteolytic cleavage by substituting the targeted residue with another residue, preferably a basic residue such as glutamine or a hydrophilic residue such as serine; by deleting the residue; or by inserting a prolyl residue immediately after the residue.

In another embodiment, any methionyl residues other than the starting methionyl residue of the signal sequence, or any residue located within about three residues N— or C-terminal to each such methionyl residue, is substituted by another residue (preferably in accord with Table 1) or deleted. Alternatively, about 1-3 residues are inserted adjacent to such sites.

The nucleic acid molecules encoding amino acid sequence mutations of the antibodies of interest are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the polypeptide on which the variant herein is based.

Oligonucleotide-mediated mutagenesis is a preferred method for preparing substitution, deletion, and insertion antibody mutants herein. This technique is well known in the art as described by Ito et al., Gene 102:67-70 (1991) and Adelman et al., DNA 2: 183 (1983). Briefly, the DNA is altered by hybridizing an oligonucleotide encoding the desired mutation to a DNA template, where the template is the single-stranded form of a plasmid or bacteriophage containing the unaltered or native DNA sequence of the polypeptide to be varied. After hybridization, a DNA polymerase is used to synthesize an entire second complementary strand of the template that will thus incorporate the oligonucleotide primer, and will code for the selected alteration in the DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75: 5765 (1978).

The DNA template can be generated by those vectors that are either derived from bacteriophage M13 vectors (e.g., the commercially available M13mp18 and M13mp19 vectors are suitable), or those vectors that contain a single-stranded phage origin of replication as described by Viera et al. Meth. Enzymol., 153: 3 (1987). Thus, the DNA that is to be mutated may be inserted into one of these vectors to generate single-stranded template. Production of the single-stranded template is described in Sections 4.21-4.41 of Sambrook et al., supra. Alternatively, single-stranded DNA template is generated by denaturing double-stranded plasmid (or other) DNA using standard techniques.

Mutations in the V_{H} and V_{L} domains may be introduced using a number of methods known in the art. These include site-directed mutagenesis strategies such as overlap extension PCR (see, e.g., Sambrook & Russell, supra; Ausubel et al., supra). Exemplary techniques and primers are provided in Examples 2 and 3.

The PCR products are subcloned into suitable cloning vectors that are well known to those of skill in the art and commercially available. Clones containing the correct size DNA insert are identified, for example, agarose gel electrophoresis. The nucleotide sequence of the heavy or light chain coding regions is then determined from double stranded plasmid DNA using the sequencing primers adjacent to the cloning site. Commercially available kits (e.g., the Sequenase® kit, United States Biochemical Corp., Cleveland, Ohio) are used to facilitate sequencing the DNA.

One of skill will appreciate that, utilizing the sequence information provided for the variable regions, nucleic acids encoding these sequences are obtained using a number of methods well known to those of skill in the art. Thus, DNA encoding the variable regions is prepared by any suitable method, including, for example, amplification techniques such as ligase chain reaction (LCR) (see, e.g., Wu & Wallace (1989) Genomics 4:560, Landegren, et al. (1988) Science 241:1077, and Barringer, et al. (1990) Gene 89:117), transcription amplification (see, e.g., Kwoh, et al. (1989) Proc. Natl Acad. Sci. USA 86:1173), and self-sustained sequence replication (see, e.g., Guatelli, et al. (1990) Proc. Natl Acad. Sci. USA 87:1874), cloning and restriction of appropriate sequences or direct chemical synthesis by methods such as the phosphotriester method of Narang, et al., (1979) Meth. Enzymol. 68:90; the phosphodiester method of Brown, et al., (1979) Meth. Enzymol. 68:109; the diethylphosphoramidite method of Beaucage, et al., (1981) Tetra. Lett. 22:1859; and the solid support method of U.S. Pat. No. 4,458,066.

The nucleic acid sequences that encode the single chain antibodies, or variable domains, are identified by techniques well known in the art (see, Sambrook, et al., supra). Briefly, the DNA products described above are separated on an electrophoretic gel. The contents of the gel are transferred to a suitable membrane (e.g., Hybond-N®, Amersham) and hybridized to a suitable probe under stringent conditions. The probe should comprise a nucleic acid sequence of a fragment embedded within the desired sequence.

If the DNA sequence is synthesized chemically, a single stranded oligonucleotide will result. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, subsequences may be cloned and the appropriate subsequences cleaved using appropriate restriction enzymes. The fragments may then be ligated to produce the desired DNA sequence.

Nucleic acids encoding monoclonal antibodies or variable domains thereof are typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding therapeutic proteins comprise a nucleic acid sequence encoding a therapeutic protein and subsequences, interspecies homologues, alleles and polymorphic variants thereof.

To obtain high level expression of a cloned gene, such as those cDNAs encoding a suitable monoclonal antibody, one typically subclones the gene encoding the monoclonal antibody into an expression vector that contains a strong promoter to direct transcription, a transcription/translation terminator, and if for a nucleic acid encoding a protein, a ribosome binding site for translational initiation. Suitable promoters are well known in the art and described, e.g., in Sambrook et al., supra and Ausubel et al., supra. Eukaryotic expression systems for mammalian cells are well known in the art and are also commercially available. Kits for such expression systems are commercially available.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

The nucleic acid comprises a promoter to facilitate expression of the nucleic acid within a cell. Suitable promoters include strong, eukaryotic promoter such as, for example promoters from cytomegalovirus (CMV), mouse mammary tumor virus (MMTV), Rous sarcoma virus (RSV), and adenovirus. More specifically, suitable promoters include the promoter from the immediate early gene of human CMV (Boshart et al., (1985) Cell 41:521) and the promoter from the long terminal repeat (LTR) of RSV (Gorman et al., (1982) Proc. Natl. Acad. Sci. USA 79:6777).

For eukaryotic expression, the construct may comprise at a minimum a eukaryotic promoter operably linked to a nucleic acid operably linked to a polyadenylation sequence. The polyadenylation signal sequence may be selected from any of a variety of polyadenylation signal sequences known in the art, such as, for example, the SV40 early polyadenylation signal sequence. The construct may also include one or more introns, which can increase levels of expression of the nucleic acid of interest, particularly where the nucleic acid of interest is a cDNA (e.g., contains no introns of the naturally-occurring sequence). Any of a variety of introns known in the art may be used.

Other components of the construct may include, for example, a marker (e.g., an antibiotic resistance gene (such,as an ampicillin resistance gene)) to aid in selection of cells containing and/or expressing the construct, an origin of replication for stable replication of the construct in a bacterial cell (preferably, a high copy number origin of replication), a nuclear localization signal, or other elements which facilitate production of the nucleic acid construct, the protein encoded thereby, or both.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

Some expression systems have markers that provide gene amplification such as thymidine kinase, hygromycin B phosphotransferase, and dihydrofolate reductase.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

Standard transfection methods are used to produce bacterial, mammalian, yeast, insect, or plant cell lines that express large quantities of the antibody or variable region domains, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the monoclonal antibody or a variable domain thereof.

After the expression vector is introduced into the cells, the transfected cells are cultured under conditions favoring expression of the monoclonal antibody or ariable domain region. The expressed protein is recovered from the culture using standard techniques known to those of skill in the art.

The monoclonal antibody or variable domain region may be purified to substantial purity by standard techniques known to those of skill in the art, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

c. Covalent Modifications of Polypeptide Variants

Covalent modifications of polypeptide variants are included within the scope of this invention. The modifications are made by chemical synthesis or by enzymatic or chemical cleavage or elaboration of the mutant antibody of the invention. Other types of covalent modifications of the polypeptide variant are introduced into the molecule by reacting targeted amino acid residues of the polypeptide variant with an organic derivatizing agent that is capable of reacting with selected side chains or the N— or C-terminal residues.

The modifications of the mutant antibody of the invention include the attachment of agents to, for example, enhance antibody stability, water-solubility, in vivo half-life and to target the antibody to a desired target tissue. Targeting the antibody preferably utilizes the covalent attachment of one or more moieties that recognize a structure on the surface of the cell to which the antibody is targeted. Exemplary targeting species include, but are not limited to, antibodies, hormones, lectins, and ligands for cell-surface receptors. Many methods are known in the art for derivatizing both the mutant antibodies of the invention and useful targeting agents. The discussion that follows is illustrative of reactive groups found on the mutant antibody and on the targeting agent and methods of forming conjugates between the mutant antibody and the targeting agent. The use of homo- and hetero-bifunctional derivatives of each of the reactive functionalities discussed below to link the mutant antibody to the targeting moiety is within the scope of the present invention.

Cysteinyl residues most commonly are reacted with agents that include α-haloacetates (and corresponding amines), such as chloroacetic acid or chloroacetamide, to give carboxymethyl or carboxyamidomethyl derivatives. Cysteinyl residues also are derivatized by reaction with bromotrifluoroketones, α-bromo-β-(5-imidozoyl)carboxylic acids, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with, for example, groups that include pyrocarbonate at pH 5.5-7.0 because this agent is relatively specific for the histidyl side chain. Para-bromophenacyl halides also are useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing a-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine site. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azo-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the alpha.-amino groups of lysine, arginine, and histidine side chains (T. E. Creighton, PROTEINS: STRUCTURE AND MOLECULAR PROPERTIES, W. H. Freeman & Co., San Francisco, pp. 79-86 (1983)), acetylation of the N-terminal amine, and amidation of any C-terminal carboxyl group.

Another type of covalent modification of the polypeptide variant included within the scope of this invention comprises altering the original glycosylation pattern of the polypeptide variant. By altering is meant deleting one or more carbohydrate moieties found in the polypeptide variant, and/or adding one or more glycosylation sites that are not present in the polypeptide variant.

Glycosylation of the mutant antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the mutant antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original polypeptide variant (for O-linked glycosylation sites). For ease, the polypeptide variant amino acid sequence is preferably altered through changes at the DNA level, particularly by mutating the DNA encoding the polypeptide variant at preselected bases such that codons are generated that will translate into the desired amino acids. The DNA mutation(s) may be made using methods described above.

Another means of increasing the number of carbohydrate moieties on the mutant antibody is by chemical or enzymatic coupling of glycosides to the polypeptide variant. These procedures are advantageous in that they do not require production of the polypeptide variant in a host cell that has glycosylation capabilities for N— or O-linked glycosylation. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston, CRC CRIT. REV. BIOCHEM., pp. 259-306 (1981).

Removal of any carbohydrate moieties present on the mutant antibody is accomplished either chemically or enzymatically. Chemical deglycosylation requires exposure of the polypeptide variant to the compound trifluoromethanesulfonic acid, or an equivalent compound. This treatment results in the cleavage of most or all sugars except the linking sugar (N-acetylglucosamine or N-acetylgalactosamine), while leaving the mutant antibody intact. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Another type of covalent modification of the polypeptide variant comprises linking the polypeptide variant to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or U.S. Pat. No. 4,179,337. The polymers are added to alter the properties of the mutant antibody or, alternatively, they serve as spacer groups between the targeting agent and the mutant antibody.

d. Preparation of the Mutant Antibody-Targeting Moiety Conjugate

The targeted mutant antibodies of the invention are exemplified in the discussion that follows by a class of antibodies of the invention that are targeted by attachment to tissue-specific antibodies. Antibodies that are reactive with surface antigens on many human cells are known in the art. In a preferred embodiment, the targeting antibody is one binding with human carcinoma cells. Antbodies-targeting moiety conjugates can be prepared by covalent modification of the antibody and the targeting agent to link them together as described in in Hellstrom et al., U.S. Pat. No. 6,020,145, for example. Alternatively, the antibody-targeting moiety conjugates can be generated as fusion proteins.

Preparation of the immunoconjugate for the present targeting system includes attachment of an enzymatic or component (AC) to an antibody and forming a stable complex without compromising the activity of either component. An exemplary strategy involves incorporation of a protected sulfhydryl onto the AC using the heterobifunctional crosslinker SPDP (n-succinimidyl-3-(2-pyridyldithio)propionate and then deprotecting the sulfhydryl for formation of a disulfide bond with another sulfhydryl on the antibody. Instead of destabilizing the antibody with reducing agents to generate free sulfhydryls, new sulfhydryls are preferably incorporated onto the mutant antibody using SPDP. In the protected form, the SPDP generated sulfhydryls on the antibody react with the free sulfhydryls incorporated onto the AC forming the required disulfide bonds. By optimizing reaction conditions, the degree of SPDP modification of each component is controlled, thus maintaining maximum activity of each component. SPDP reacts with primary amines and the incorporated sulfhydryl is protected by 2-pyridylthione.

If SPDP should affect the activities of either the antibody (e.g., the moiety binding to the reactive chelate) or the AC, there are a number of additional crosslinkers such as 2-iminothiolane or N-succinimidyl S-acetylthioacetate (SATA), available for forming disulfide bonds. 2-iminothiolane reacts with primary amines, instantly incorporating an unprotected sulfhydryl onto the protein. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategy is exemplary and not limiting of linkers of use in the invention. Other crosslinkers are available that can be used in different strategies for crosslinking the targeting agent to the mutant antibody. For example, TPCH(S-(2-thiopyridyl)-L-cysteine hydrazide and TPMPH ((S-(2-thiopyridyl) mercapto-propionohydrazide) react at the carbohydrate moieties of glycoproteins that have been previously oxidized by mild periodate treatment, thus forming a hydrazone bond between the hydrazide portion of the crosslinker and the periodate generated aldehydes. The placement of this crosslinker on the antibody is beneficial since the modification is site-specific and will not interfere with the antigen binding site of the antibody. TPCH and TPMPH introduce a 2-pyridylthione protected sulfhydryl group onto the antibody, which can be deprotected with DTT and then subsequently used for conjugation, such as forming disulfide bonds between components.

If disulfide bonding is found unsuitable for producing stable conjugates, other crosslinkers may be used that incorporate more stable bonds between components. The heterobifunctional crosslinkers GMBS (N-gamma-maleimidobutyryloxy)succinimide) and SMCC (succinimidyl 4-(N-maleimido-methyl)cyclohexane) react with primary amines, thus introducing a maleimide group onto the component. This maleimide group can subsequently react with sulfhydryls on the other component, which can be introduced by previously mentioned crosslinkers, thus forming a stable thioether bond between the components. If steric hindrance between components interferes with either component's activity, crosslinkers can be used which introduce long spacer arms between components and include derivatives of some of the previously mentioned crosslinkers (i.e., SPDP). Thus there is an abundance of suitable crosslinkers, which are useful; each of which is selected depending on the effects it has on optimal immunoconjugate production.

A variety of reagents are used to modify the components of the conjugate with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (J. S. Holcenberg, and J. Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred useful crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

e. Preferred Specific Sites in Crosslinking Reagents

1. Amino-Reactive Groups

In one preferred embodiment, the sites are amino-reactive groups. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides.

NHS esters react preferentially with the primary (including aromatic) amino groups of the affinity component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with the amine groups of the conjugate components. At a pH between 7 and 10, imidoesters react only with primary amines. Primary amines attack imidates nucleophilically to produce an intermediate that breaks down to amidine at high pH or to a new imidate at low Ph. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines of the conjugate components (e.g., ε-amino group of lysine residues). Glutaraldehyde, however, displays also reactivity with several other amino acid side chains including those of cysteine, histidine, and tyrosine. Since dilute glutaraldehyde solutions contain monomeric and a large number of polymeric forms (cyclic hemiacetal) of glutaraldehyde, the distance between two crosslinked groups within the affinity component varies. Although unstable Schiff bases are formed upon reaction of the protein amino groups with the aldehydes of the polymer, glutaraldehyde is capable of modifying the affinity component with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites of the conjugate components, but reaction with the amino groups is the most important, resulting in a stable sulfonamide linkage.

2. Sulfhydryl-Reactive Groups

In another preferred embodiment, the sites are sulfhydryl-reactive groups. Useful non-limiting examples of sulfhydryl-reactive groups include maleimides, alkyl halides, pyridyl disulfides, and thiophthalimides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form also disulfides.

3. Guanidino-Reactive Groups

In another embodiment, the sites are guanidino-reactive groups. A useful non-limiting example of a guanidino-reactive group is phenylglyoxal. Phenylglyoxal reacts primarily with the guanidino groups of arginine residues in the affinity component. Histidine and cysteine also react, but to a much lesser extent.

4. Indole-Reactive Groups

In another embodiment, the sites are indole-reactive groups. Useful non-limiting examples of indole-reactive groups are sulfenyl halides. Sulfenyl halides react with tryptophan and cysteine, producing a thioester and a disulfide, respectively. To a minor extent, methionine may undergo oxidation in the presence of sulfenyl chloride.

5. Carboxol-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage (Yamada et al., Biochemistry 20: 4836-4842, 1981) teach how to modify a protein with carbodiimde.

f. Preferred Nonspecific Sites in Crosslinking Reagents

In addition to the use of site-specific reactive moieties, the present invention contemplates the use of non-specific reactive groups to link the mutant antibody to the targeting moiety. Non-specific groups include photoactivatable groups, for example. In another preferred embodiment, the sites are photoactivatable groups. Photoactivatable groups, completely inert in the dark, are converted to reactive species upon absorption of a photon of appropriate energy. In one preferred embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferrred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C-H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

g. Homobifunctional Reagents

1. Homobifunctional Crosslinkers Reactive with Primary Amines

Synthesis, properties, and applications of such reagents are described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxy-carbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidyl-propionate (DSP), and dithiobis(sulfosuccinimidylpropionate (sulfo-DSP). Preferred, non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)-dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of homobifunctional arylhalides include 1,5-difluoro-2,4-dinitrobenzene (DFDNB), and 4,4′-difluoro-3,3′-dinitrophenyl-sulfone.

Preferred, non-limiting examples of homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of homobifunctional aromatic sulfonyl chlorides include phenol-2,4-disulfonyl chloride, and .alpha.-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether. Preferred, non-limiting examples of homobifunctional pyridyl disulfides include 1,4-di->3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(b-bromoethyl)benzylamine, N,N′-di(bromoacetyl)phenylthydrazine, and 1,2-di(bromoacetyl)amino-3-phenylpropane.

3. Homobifunctional Photoactivatable Crosslinkers

Synthesis, properties, and applications of such reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional photoactivatable crosslinker include bis-b-(4-azidosalicylamido)ethyldisulfide (BASED), di-N-(2-nitro-4-azidophenyl)-cystamine-S,S-dioxide (DNCO), and 4,4′-dithiobisphenylazide.

h. Hetero-Bifunctional Reagents

1. Amino-Reactive Hetero-Bifunctional Reagents with a Pyridyl Disulfide Moiety

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-a-methyl-α-(2-pyridyldithio)toluene (SMPT), and sulfosuccinimidyl 6-a-methyl-α-(2-pyridyldithio)toluamidohexanoate (sulfo-LC-SMPT).

2. Amino-Reactive Hetero-Bifunctional Reagents with a Maleimide Moiety

Synthesis, properties, and applications of such reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (BMPS), N-γ-maleimidobutyryloxysuccinimide ester (GMBS)N-γ-maleimidobutyryloxysulfo succinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)-cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive Hetero-Bifunctional Reagents with an Alkyl Halide Moiety

Synthesis, properties, and applications of such reagents are described in the literature Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)-methyl)-cyclohexane-1-carbonyl)aminohexanoate (SIACX), and succinimidyl-4((iodoacetyl)-amino)methylcyclohexane-1-carboxylate (SIAC).

A preferred example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety for primary amino groups is controlled by the reaction temperature (McKenzie et al., Protein Chem. 7: 581-592 (1988)).

Preferred, non-limiting examples of hetero-bifunctional reagents with an alkyl halide moiety and an amino-reactive p-nitrophenyl ester moiety include p-nitrophenyl iodoacetate (NPIA).

4. Photoactivatable Arylazide-Containing Hetero-Bifunctional Reagents with a NHS Ester Moiety

Synthesis, properties, and applications of such reagents are described in the literature. Preferred, non-limiting examples of photoactivatable arylazide-containing hetero-bifunctional reagents with an amino-reactive NHS ester include N-hydroxysuccinimidyl-4-azidosalicylic acid (NHS-ASA), N-hydroxysulfosuccinimidyl-4-azidosalicylic acid (sulfo-NHS-ASA), sulfosuccinimidyl-(4-azidosalicylamido)hexanoate (sulfo-NHS-LC-ASA), N-hydroxysuccinimidyl N-(4-azidosalicyl)-6-aminocaproic acid (NHS-ASC), N-hydroxy-succinimidyl-4-azidobenzoate (HSAB), N-hydroxysulfo-succinimidyl-4-azidobenzoate (sulfo-HSA8), sulfosuccinimidyl-4-(p-azidophenyl)butyrate (sulfo-SAPB), N-5-azido-2-nitrobenzoyloxy-succinimide (ANB-NOS), N-succinimidyl-6-(4′-azido-2′-nitrophenyl-amino)hexanoate (SANPAH), sulfosuccinimidyl-6-(4′-azido-2′-nitrophenylamino)-hexanoate (sulfo-SANPAH), N-succinimidyl 2-(4-azidophenyl)dithioacetic acid (NHS-APDA), N-succinimidyl-(4-azidophenyl)1,3′-dithiopropionate (SADP), sulfosuccinimidyl-(4-azidophenyl)-1,3′-dithiopropionate (sulfo-SADP), sulfosuccinimidyl-2-(m-azido-o-nitrobenzamido)ethyl-1,3′-dithiopropionate (SAND), sulfosuccinimidyl-2-(p-azidosalicylamido)-ethyl-1,3′-dithiopropionate (SASD), N-hydroxysuccinimidyl 4-azidobenzoylglycyltyrosine (NHS-ABGT), sulfosuccinimidyl-2-(7-azido-4-4-methylcoumarin-3-acetamide)ethyl-1,3′-dithiopropionate (SAED), and sulfosuccinimidyl-7-azido-4-methylcoumarin-3-acetate (sulfo-SAMCA).

Other cross-linking agents are known to those of skill in the art (see, for example, Pomato et al., U.S. Pat. No. 5,965,106.

i. Linker Groups

In addition to the embodiments set forth above, wherein the cross-linking moiety is attached directly to a site on the mutant antibody and on the targeting moiety, the present invention also provides constructs in which the cross-linking moiety is bound to a site present on a linker group that is bound to either the mutant antibody or the targeting moiety or both. In certain embodiments, it is advantageous to tether the mutant antibody and the targeting moiety by a group that provides flexibility and increases the distance between the mutant antibody and the targeting moiety. Using linker groups, the properties of the oligonucleotide adjacent to the stabilizing moiety can be modulated. Properties that are usefully controlled include, for example, hydrophobicity, hydrophilicity, surface-activity and the distance of the targeting moiety from the oligonucleotide.

In an exemplary embodiment, the linker serves to distance the mutant antibody from the targeting moiety. Linkers with this characteristic have several uses. For example, a targeting moiety held too closely to the mutant antibody may not interact with its complementary group, or it may interact with too low of an affinity. Similarly, a targeting moiety held to closely to the mutant antibody may prevent the antibody from binding the reactive chelate. Thus, it is within the scope of the present invention to utilize linker moieties to, inter alia, vary the distance between the mutant antibody and the targeting moiety.

In yet a further embodiment, the linker group is provided with a group that can be cleaved to release the mutant antibody from the targeting moiety. Many cleaveable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover, a broad range of cleavable, bifunctional (both homo- and hetero-bifunctional) linker groups are commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to their being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

In the discussion set forth above, examples are set forth in which the linkers are located between the mutant antibody and a targeting moiety. Those of skill will appreciate that linkers can also be interposed between the chelating agent and the reactive functional group having reactivity complementary to the reactivity of the reactive site on the mutant antibody.

j. Fusion Proteins

In a preferred form, the antibodies are recombinantly produced as fusion proteins with a second, antitumor antibody that acts to target the fusion protein to an antigen of a targeted tumor. Dozens of antitumor antigens and antibodies against them are known in the art, many of which are in clinical trials. Examples include AMD-Fab, LDP-02, aCD-11a, aCD-18, a-VEGF, a-IgE, and Herceptin, from Genentech, ABX-CBL, ABX-EGF, and ABX-IL8, from Abgenix, and aCD3, Smart 195 and Zenepax from Protein Design Labs. In preferred forms, the antibody is HMFG1, L6, or Lym-1, with Lym-1 being the most preferred. In preferred embodiments, an scFv or dsFv form of the antibody is employed. Formation of scFvs and dsFvs is known in the art. Formation of a scFv of Lym-1, for example, is taught Bin Song et al., Biotechnol Appl Biochem 28(2):163-7 (1998). See, also Cancer Immunol. Immunother. 43: 26-30 (1996). The two antibodies can be linked directly or, more commonly, are connected by a short peptide linker, such as Gly₄Ser repeated 3 times.

2. The Chelates

In addition to the mutant antibodies described in detail above, the invention also provides reactive chelates that are specifically recognized by the antibody antigen recognition domain and which form covalent bonds with the reactive group on the mutant antibody.

In an exemplary embodiment, there is provided a metal chelate having four nitrogen atoms that is recognized by the antigen recognition domain of a mutant antibody. The antibody includes a reactive site not present in the wildtype of the antibody and the reactive site is in a position proximate to or within the antigen recognition domain.

In a preferred embodiment, the chelate includes a substituted or unsubstituted ethyl bridge that covalently links at least two of the nitrogen atoms. An exemplary ethyl bridge is shown in the formula below: 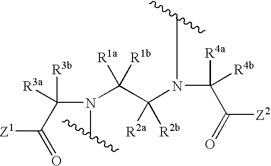
wherein Z ¹ and Z² are members independently selected from OR and NR³R⁴, in which R³ and R⁴ are members independently selected from H, substituted or unsubstituted alkyl and substituted or unsubstituted heteroalkyl. The symbols R^{1a}, R^{1b}, R^{2a}, R^{2b}, R^{3a}, R^{3b}, R^{4a} and R^{4b} represent members independently selected from H, substituted or unsubstituted alkyl, substituted or unsubstituted heteroalkyl, substituted or unsubstituted aryl and linker moieties.

In another exemplary embodiment, the chelate has the formula: 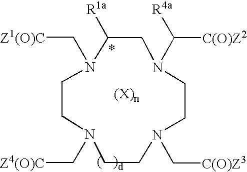
wherein Z¹, Z², Z³ and Z⁴ are members independently selected from OR¹ and NR¹R², in which R¹ and R² are members independently selected from H, substituted or unsubstituted alkyl and substituted or unsubstituted heteroalkyl. The symbol X represents a member selected from a lanthanide, an actinide, an alkaline earth metal, a group Ilfb transition metal, or a metal. The symbol n represents 0 or 1; and d is 1 or 2. In a preferred embodiment, the carbon atom marked * is of S configuration.

In another exemplary embodiment, the chelate includes a moiety having the formula: 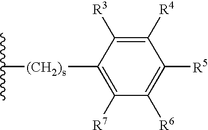
wherein R³, R⁴, R⁵, R⁶ and R⁷ are members independently selected from H, halogen, NO₂, CN, X¹R⁸, NR⁹R¹⁰, and C(X²)R¹¹. The symbol X¹ represents a member selected from O, NH and S. The symbols R⁸ and R⁹ are members independently selected from H, substituted or unsubstituted alkyl, substituted or unsubstituted heteroalkyl and C(Z³)R¹², in which X³ is a member selected from O, S and NH. R¹² is a member selected from substituted or unsubstituted alkyl, substituted or unsubstituted heteroalkyl and OR¹³, in which R¹³ is a member selected from substituted or unsubstituted alkyl, substituted or unsubstituted heteroalkyl, substituted or unsubstituted aryl and substituted or unsubstituted heteroaryl. The symbol R¹⁰ is a member selected from H, substituted or unsubstituted alkyl, substituted or unsubstituted heteroalkyl and OH, and R⁹ and R¹⁰, taken together are optionally (═C═S). X² is a member selected from O, S and NH. In some embodiments, R¹⁰ is —C(O)—CHCH². The symbol R¹¹ represents a member selected from H, halogen, substituted or unsubstituted alkyl, substituted or unsubstituted heteroalkyl, OR¹⁴, NR¹⁵R¹⁶R¹⁴ is a member selected from H, substituted or unsubstituted alkyl, substituted or unsubstituted heteroalkyl, and C(O)R¹⁷. R¹⁷ is a member selected from substituted or unsubstituted alkyl and substituted or unsubstituted heteroalkyl; and R¹⁵ and R¹⁶ are members independently selected from H, substituted or unsubstituted alkyl and substituted or unsubstituted heteroalkyl.

In practicing the present invention, the structure of the metal binding portion of the chelate is selected from an array of structures known to complex metal ions. Exemplary chelating agents of use in the present invention include, but are not limited to, reactive chelating groups capable of chelating radionuclides include macrocycles, linear, or branched moieties. Examples of macrocyclic chelating moieties include polyaza- and polyoxamacrocycles, polyether macrocycles, crown ether macrocycles, and cryptands (see, e.g., Synthesis of Macrocycles: the Diesgn of Selective Complexing Agents (Izatt and Christensen ed., 1987) and The Chemistry of Macrocyclic Ligand Complexes (Lindoy, 1989)). Examples of polyazamacrocyclic moieties include those derived from compounds such as 1,4,7,10-tetraazacyclododecane-N,N′,N″,N′″-tetraacetic acid (“DOTA”); 1,4,7,10-tetraazacyclotridecane-N,N′,N″,N′″-tetraacetic acid (“TRITA”); 1,4,8,11-tetraazacyclotetradecane-N,N′,N″,N′″-tetraacetic acid (“TETA”); and 1,5,9,13-tetraazacyclohexadecane-N,N′,N″,N′″-tetraacetic acid (abbreviated herein abbreviated as HETA). In a presently preferred embodiment, the chelating agent includes four nitrogen atoms. Other embodiments in which the chelate includes oxygen atoms or mixtures of oxygen and nitrogen atoms are within the scope of the present invention.

Chelating moieties having carboxylic acid groups, such as DOTA, TRITA, HETA, and HEXA, may be derivatized to convert one or more carboxylic acid groups to reactive groups. Alternatively, a methylene group adjacent to an amine or a carboxylic acid group can be derivatized with a reactive functional group. Additional exemplary chelates of use in the present invention are set forth in Meares et al., U.S. Pat. No. 5,958,374.

The preparation of chelates useful in practicing the present invention is accomplished using art-recognized methodologies or modifications thereof. In a preferred embodiment of the invention, a reactive derivative of DOTA is used. Preparation of DOTA is described in, e.g., Moi et al., J. Am. Chem. Soc. 110:6266-67 (1988) and Renn and Meares, Bioconjugate Chem. 3:563-69 (1992).

The chelate that is linked to the antibody or growth factor targeting agent will, of course, depend on the ultimate application of the invention. Where the aim is to provide an image of the tumor, one will desire to use a diagnostic agent that is detectable upon imaging, such as a paramagnetic, radioactive or fluorogenic agent. Many diagnostic agents are known in the art to be useful for imaging purposes, as are methods for their attachment to antibodies (see, e.g., U.S. Pat. Nos. 5,021,236 and 4,472,509, both incorporated herein by reference). In the case of paramagnetic ions, one might mention by way of example ions such as chromium (III), manganese (II), iron (III), iron (II), cobalt (TI), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III). Moreover, in the case of radioactive isotopes for therapeutic and/or diagnostic application, presently preferred isotopes include iodine¹³¹, iodine¹²³, technicium^{99m}, indium¹¹¹, rhenium¹⁸⁸, rhenium186, gallium⁶⁷, copper⁶⁷, yttrium⁹⁰, iodine¹²⁵ or astatine²¹¹.

Antibody-Chelate Bond Formation

In general, after the formation of the antibody-antigen (chelate) complex, the reactive chelate and mutant antibody of the invention are linked together through the use of reactive groups, which are typically transformed by the linking process into a new organic functional group or unreactive species. The chelate reactive functional group(s), is located at any position on the metal chelate. Reactive groups and classes of reactions useful in practicing the present invention are generally those that are well known in the art of bioconjugate chemistry. Currently favored classes of reactions available with reactive chelates are those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John,Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Useful reactive pendant functional groups include, for example:
(a) carboxyl groups and various derivatives thereof including, but not limited to, N-hydroxysuccinimide esters, N-hydroxybenztriazole esters, acid halides (e.g., I, Br, Cl), acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble the reactive chelates. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

B. The Methods

In addition to the compositions of the invention, the present invention provides methods for using the compositions. Thus, in a fourth aspect, the invention provides a method of using the compositions of the invention to treat a patient for a disease or condition (e.g., cancer or autoimmune diseases, such as diabetes, arthritis, systemic lupus erythematosus) or to diagnose a condition or disease. The method comprising the steps of: (a) administering to the patient a mutant antibody comprising; (i) an antigen recognition domain that specifically binds to the metal chelate; (ii) a reactive site not present in the wild-type of the antibody and, wherein the reactive site is in a position proximate to or within the antigen recognition domain; and (iii) a targeting moiety that binds specifically to a cell by binding with a surface group (e.g., cell surface receptors and cell surface antigens), thereby forming a complex between the mutant antibody and the cell. Following step (a), the metal chelate is administered to the patient. The metal chelate comprises a reactive functional group having a reactivity complementary to the reactivity of the reactive site of said antibody. Thus, the chelate and the antibody bind to form an antibody-antigen (chelate) pair, the reactive groups of which subsequently react to form a covalent bond between the antibody and the antigen. As discussed above, the techniques relevant to raising antibodies and preparing chelates useful in the above-recited method are well known in the art.

The present invention provides antibodies raised against essentially any chelate of any metal ion. In a preferred embodiment, the antibody used for pretargeting is 2D12.5, a monoclonal antibody that binds metal chelates of DOTA and to similar structures.

In addition to the method described above, the present invention also provides a method in which the tissue is pretargeted with an antibody of the invention. This pretargeting method of treating a patient with a metal chelate comprises the steps of: (a) administering to a patient a mutant antibody of the invention. In some embodiments, the antibody comprises a recognition moiety that binds specifically with a compenent on the surface of a cell, thereby forming a complex between the cells and the antibody. After the antibody has localized in the desired tissue, a metal chelate is administered to the patient. The chelate specifically binds to the antibody of the invention, forming an antibody-metal chelate complex.

The mutant antibody comprises: (i) a antigen recognition domain that specifically binds to the metal chelate; (ii) a reactive site not present in the wild-type of the antibody (the reactive site is in a position proximate to or within the antigen recognition domain); and (iii) a recognition moiety that binds specifically with the pretargeting reagent, thereby forming a complex between the pretargeting reagent and the mutant antibody. After the pretargeting reagent has localized in the desired tissue, following step (b), a metal chelate is administered to the patient. The chelate specifically binds to the mutant antibody of the invention, forming an antibody-antigen complex. Moreover, the chelate comprises a reactive functional group having a reactivity that is preferably complementary to the reactivity of the reactive site on the mutant antibody such that a covalent bond is formed via reaction of the reactive functional group of the chelate and the reactive site of the mutant antibody. After the antibody-antigen complex is formed, the reactive site of the antibody and that of the metal chelate react to form a covalent bond between the mutant antibody and the metal chelate.

Pretargeting methods have been developed to increase the target:background ratios of the detection or therapeutic agents. Examples of pre-targeting and biotin/avidin approaches are described, for example, in Goodwin et al., U.S. Pat. No. 4,863,713; Goodwin et al., J. Nucl. Med. 29: 226 (1988); Hnatowich et al., J. Nucl. Med. 28: 1294 (1987); Oehr et al., J. Nucl. Med. 29: 728 (1988); Klibanov et al., J. Nucl. Med. 29: 1951 (1988); Sinitsyn et al., J. Nucl. Med. 30: 66 (1989); Kalofonos et al., J. Nucl. Med. 31: 1791 (1990); Schechter et al., Int. J. Cancer 48:167 (1991); Paganelli et al., Cancer Res. 51:5960 (1991); Paganelli et al, Nucl. Med. Commun. 12: 211 (1991); Stickney et al., Cancer Res. 51: 6650 (1991); and Yuan et al., Cancer Res. 51:3119, 1991; all of which are incorporated by reference herein in their entirety.

In both of the above-described aspects of the invention, it is preferable that a significant proportion of the antibodies used remain on the cell surface to be accessible to a later introduced moiety containing the radioactive agent. Thus, it is generally preferable to choose antigens that are not rapidly endocytosed or otherwise internalized by the cell upon antibody binding. Preferably, at least one-quarter of the bound antibody should remain on the cell surface and not internalized. In some cases, however, even less of the bound antibody may remain on the cell surface. For example, for a particular tumor type, an antigen which has a high rate of internalization may still be used for pretargeting if there is no known antigen with a lower internalization rate (or for which an antibody is available) with which to image tumor locations. The suitability of a particular antigen can be determined by simple assays known in the art.

1. Clearing Agents

Clearing agents known in the art may be used in accordance with the present invention. In a preferred embodiment, the clearing agent is an antibody that binds the binding site of the targeting species, where the targeting species can be an antibody, an antigen binding antibody fragment or a non-antibody targeting species. In a more preferred embodiment, the clearing agent is a MAb that is anti-idiotypic to the MAb of the conjugate used in the first step, as described in U.S. application Ser. No. 08/486,166. In another preferred embodiment, the clearing agent is substituted with multiple residues of carbohydrate, such as galactose, which allow the clearing agent to be cleared quickly from circulation by asialoglycoprotein receptors in the liver.

In a more preferred embodiment, the clearing agent is an anti-idiotypic MAb substituted with galactose and small numbers of biotin residues. Different purposes are being accomplished here. The anti-idiotypic MAb clears the first antibody conjugate (radioiodinated MAb-SAv) from circulation and deposits this into the hepatocytes. Because the anti-idiotypic MAb binds to the Mab binding region of the first antibody, it does not remove first antibody conjugate already localized at the tumor sites.

The multiple galactose substitution ensures the rapid clearance of the anti-idiotypic MAb into the liver hepatocytes, usually within minutes. Because the anti-idiotypic MAb is galactosylated and cleared rapidly, it does not have a chance to competitively remove the tumor-localized first antibody conjugate from the tumor over time. Also, there is very little myelotoxicity since almost all circulating radioactivity has been removed from the blood.

2. Blocking Agents

Blocking agents known in the art may be used in accordance with the present invention. In an exemplary embodiment, a blocking agent (e.g., an agent that reduces the uptake of metal chelates into unwanted targets), metal chelate conjugate (see, e.g., Karacay et al., Bioconjugate Chem. 13:1054-1070 (2002)). Suitable blocking agents include, for example, DOTA-conjugated to bovine serum albimun (BSA), DOTA conjugated to immunoglobulin, and DOTA conjugated to peptides.

The materials, methods and devices of the present invention are further illustrated by the examples that follow. These examples are offered to illustrate, but not to limit the claimed invention.

### EXAMPLES

Example 1 sets forth the use of rational computer-aided design to develop mutants of the monoclonal antibody 2D12.5. Example 2 describes construction of chimeric constructs comprising the variable domain of 2D12.5 and human antibody specific for tetanus toxoid. Example 3 demonstrates that the monoclonal antibody 2D12.5 has broad specificity and high affinity for all rare earth metal DOTA complexes.

### Example 1

This Example sets forth the use of rational computer-aided design to develop mutants of the monoclonal antibody 2D12.5.

Evaluation of the crystal structure of 2D12.5 bound to its hapten, Y-DOTA in conjunction with molecular modeling software (InsightII, Biosym/MSI) identified two specific side-arm orientations of the chelate in the binding pocket. This observation led to the design and engineering of four separate cysteine mutants (three heavy chain and one light chain). Specifically, cysteine residues were substituted at positions 53, 54, and 55 (positions 54, 55, and 56 if the Kabat standard numbering system is used) of the heavy chain variable domain and position 53 (position 54 if the Kabat standard numbering system is used) of the light chain variable domain. These mutants can conveniently be used in experiments to evaluate the ability of the mutants to irreversible bind suitably derived electrophilic chelates. Additional mutants can conveniently be generated based on the evaluation of the crysal structure of 2D12.5 bound to its hapten.

### Example 2

The following example describes the methodology used to prepare chimeric heavy and light chain Fab genes for expression in Drosophila Schneider (S2) cells. In all, six different chimeric heavy chain constructs were prepared. The first was the native heavy chain that was composed of the 2D12.5 mAb's variable domain fused with the CH1 of a human anti-tetanus toxoid antibody. The native variable domain contained a N-linked glycosylation site at position 87. This glycocsylation site was removed by engineering a N87D mutant (FR3). This N87D mutant was the “native” heavy chain that was used to construct the three heavy chain cysteine mutants: G53C, G54C and G55C, which are all part of CDR2. The native chimeric light chain and only cysteine mutant (N53C) were also constructed. The N53C mutation is located on CDR2 of the light chain.

As explained above, after inspection of the crystal structure we chose to introduce cysteine residues at positions 53, 54, and 56 (positions 54, 55, and 56 if the Kabat standard numbering system is used) of the heavy chain variable domain and position 53 (position 54 if the Kabat standard numbering system is used) of the light chain variable domain.

2D12.5 hybridoma cells were grown in RPMI 1640 supplemented with 10% FCS and used as a source of genetic template. Poly A mRNA was extracted using methods known to those skilled in the art. Complementary DNA synthesis and PCR amplification of the variable domain genes was accomplished using Novagen's Mouse Ig-Primer kit which incorporates degenerate 3′ constant domain primers specific to mouse IgG genes. Double stranded DNA was obtained from cDNA using degenerate 5′ and 3′ primers provided in the Mouse Ig-Primer kit. The heavy and light chain variable genes, each with an unpaired 3′ terminal A, were cloned separately into a pT7Blue T-vector and sequenced. The variable domains were then used to prepare expression constructs.

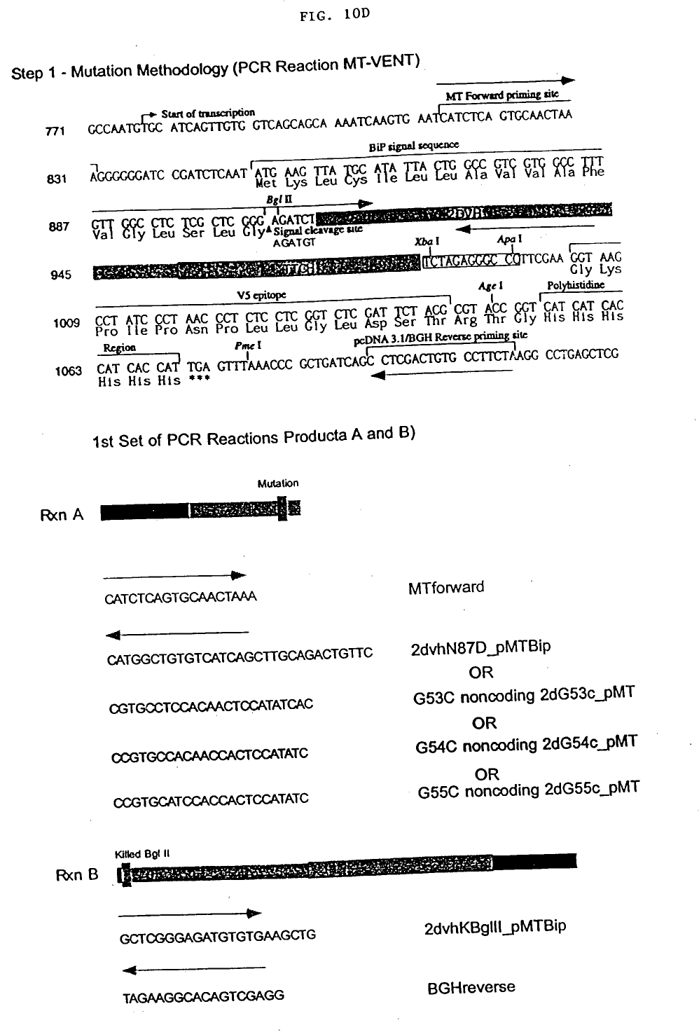

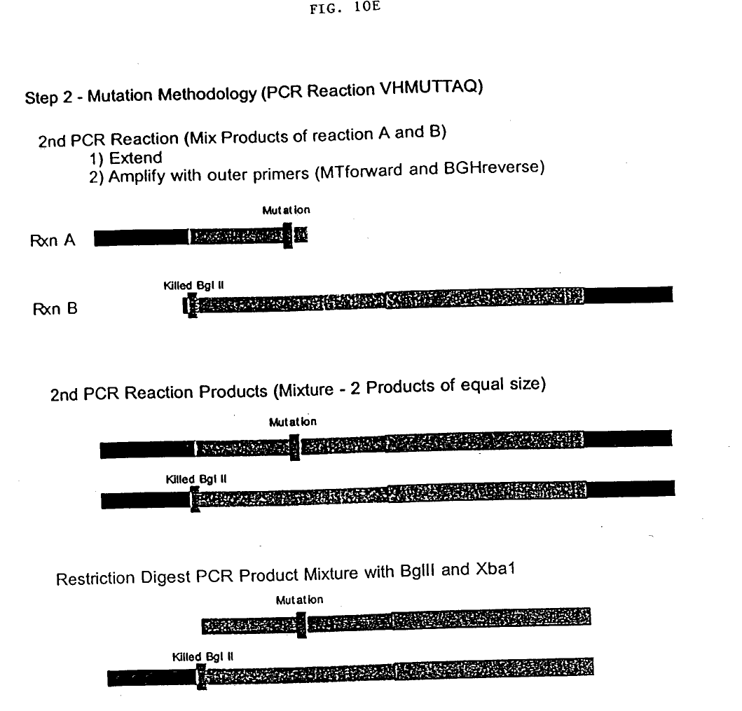

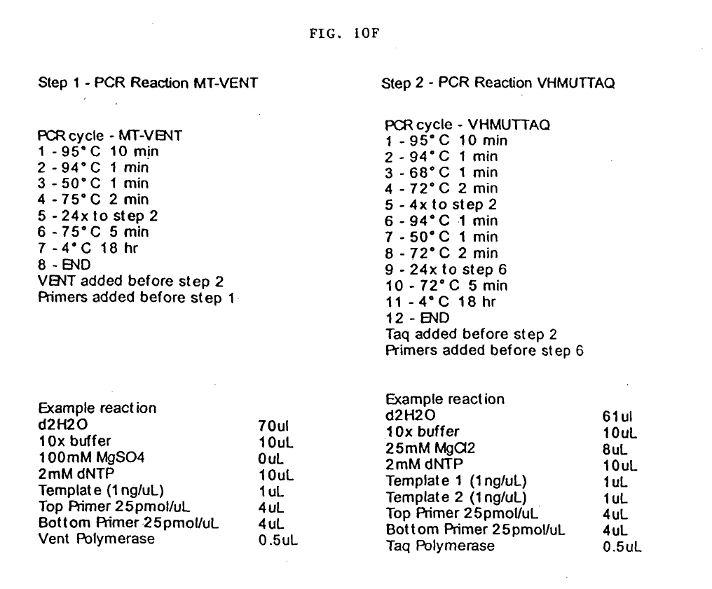

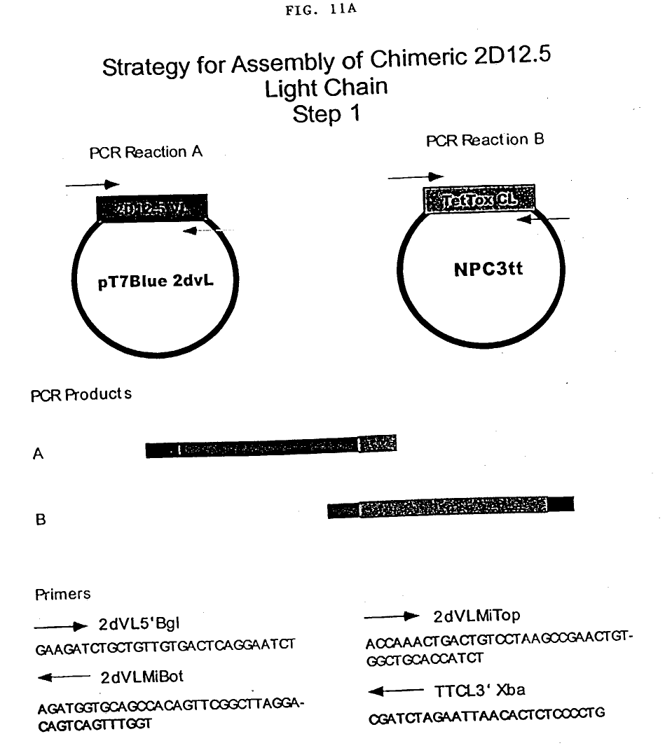

Assembly of the chimeric constructs is shown in FIG. 10 (heavy chain) and FIG. 11 (light chain). Chimeric constructs of the murine 2D12.5 variable (light and heavy) domains and human anti-tetanus toxoid antibody CL and CH1 domains were assembled by two-step overlap extension (see, e.g., Pont-Kingdon, Biotechniques 16:1010-1011 (1994) and erratum 18:636 (1995)) and as shown in FIGS. 10 and 11. A BglII restriction site was introduced onto the 5′ end of heavy and light chain genes and a XbaI restriction site was introduced onto the 3′ end of the tetanus toxin CH1 chain or C_{L}κ chain during overlap extension, and were used to introduce each chimeric gene construct into the pMT/BipNV5/His plasmid cassette for propagation in E. coli and expression in Drosophila S2 cells. Heavy and light chain genes were placed into separate plasmids. Site directed substitution of aspartic acid at position 87 (N87D) of the heavy chain was accomplished as described in Ito et al., Gene 102: 67-70 (1991). Site directed substitution of cysteine at positions 53 (G53C), 54 (G54C), and 55 (G55C) of the heavy chain and position 53 (N53C) of the light chain was also accomplished as described in Ito et al., 1991, supra. Mutations were prepared using MT and BGH sequencing primers as well as a killed BglII primer, and site-specific mutation primers. As shown in FIGS. 10 and 11, four primers and two PCR steps are required to install a mutation in a gene by this method. The primers for site-directed substitution at positions 53 (G53C), 54 (G54C), and 55 (G55C) of the heavy chain are shown in FIG. 10 and the primers for site-directed substitution at position position 53 (N53C) of the light chain is shown in FIG. 11, as are exemplary PCR conditions and reaction mixtures.

Heavy and light chain containing plasmids were cotransfected into Drosophila S2 cells using precipitating calcium phosphate. Cells were induced using 500 μM CUSO₄. Stable cell lines were produced by cotransfecting a plasmid containing the hygromycin B phosphotransferase gene along with heavy and light chain DNA. Selection proceeded for 3-4 weeks post-transfection with 300 μg/mL hygromycin B.

Each of the heavy chains were cotransfected with the native light chain in Drosophila S2 cells. Also, the N87D heavy chain was cotransfected with the N53C light chain. Stably transfected Drosophila S2 cells were induced (native as well as 4 cysteine mutants), and the media was assayed for gene expression by denaturing, nonreducing SDS gel separation followed by Western Blot analysis. Goat anti-human-κ and anti-V5 epitope antibodies (alkaline phosphatase (AP) conjugates) were used to detect for light and heavy chains, respectively. It is clear from the blots that there is heterogeneous glycosylation of the heavy chain. The glycoprotein bands are not present in heavy chains incorporating the N87D mutation, yielding a homogeneous product that is preferable for future applications.

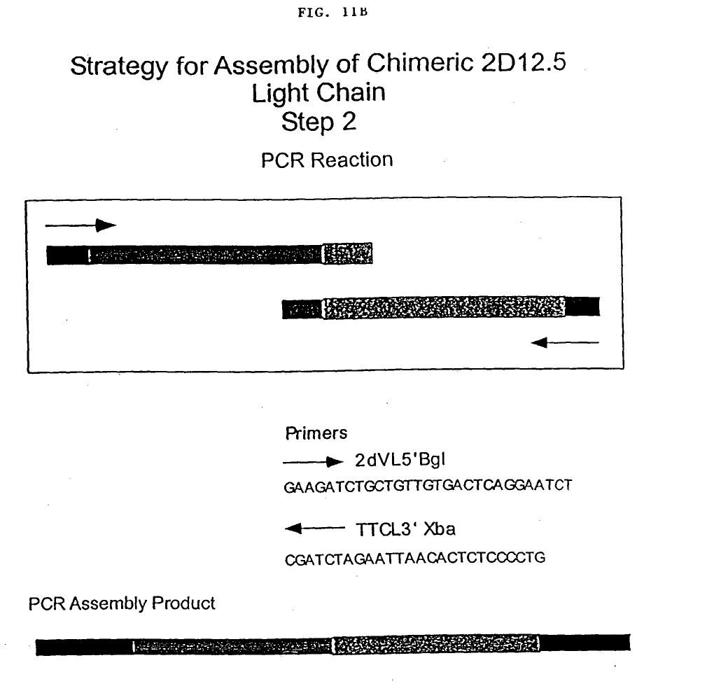

Stably transfected Drosophila S2 cells expressing the chimeric 2D12.5 FaB gene products (native and site-directed cysteine mutants) were evaluated for their ability to bind Y-DOTA. Binding curves were determined from non-competitive ELISA assays incorporating dilutions of media containing expressed gene products. The relative amound of expressed chimeric Fab were measured using anti-V5 epitope-HRP conjugate and a visible TMB (3,3′,5,5′-tetramethyl benzidine) substrate. The results are shown in FIG. 12.

### Example 3

To determine the metal selectivity of the antibody 2D12.5, a competitive immunoassays was used to measure the binding constants of multiple metal-DOTA complexes relative to Y³⁺-DOTA.

We examined the monoclonal antibody 2D12.5, initially developed to bind specifically to Y-DOTA for targeted radiotherapy (see, e.g., Goodwin et al., Cancer Res. 54: 5937-5946 (1994)), in order to determine the scope of its activity. To assess the metal selectivity of antibody 2D12.5, a competitive immunoassay to measure the binding constants of various metal-DOTA complexes relative to the original Y³⁺ complex was developed (FIG. 12). Briefly, 2D12.5 was incubated at 37° C. in the presence of immobilized Y-DOTA and a soluble metal-DOTA competitor. The metal-DOTA concentration was varied from μM to pM in order to determine the relative binding affinity of 2D12.5 for each metal chelate in comparison to Y-DOTA. Binding was measured by standard methods known to those of skill in the art. The DOTA analog used to evaluate binding was [S]-2-(p-nitrobenzyl)-DOTA, which is similar to the original antigen.

We found that 2D12.5 binds not only Y-DOTA but also DOTA complexes of all the lanthanides. Surprisingly, some metal chelates such as Gd-DOTA bind more tightly than the original Y³⁺ complex; overall, the dissociation constants fall within a factor of 3 above or below the Kd=10 nM value for Y-DOTA. Other antibodies that bind metal chelates do so with a strong preference for one or possibly two metals (see, e.g., Love et al., Biochemistry 32: 10950-10959 (1993) and Khosraviani et al., Bioconjugate Chem. 11: 267-277 (2000)).

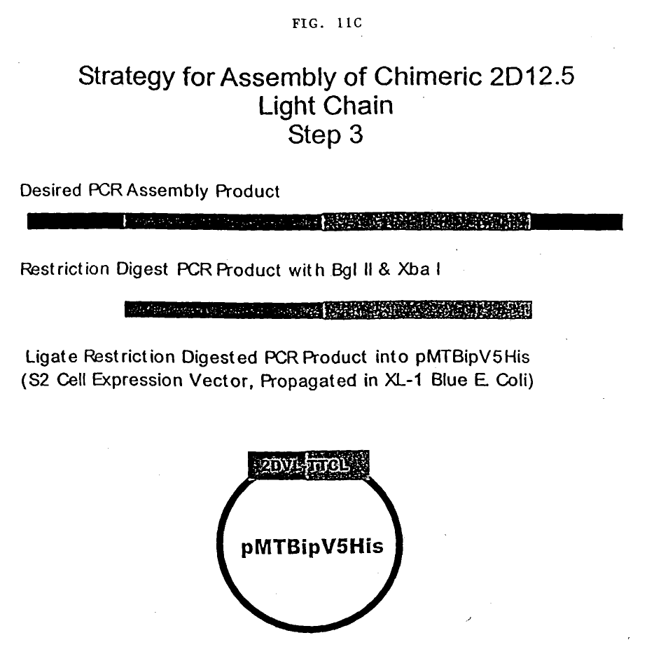

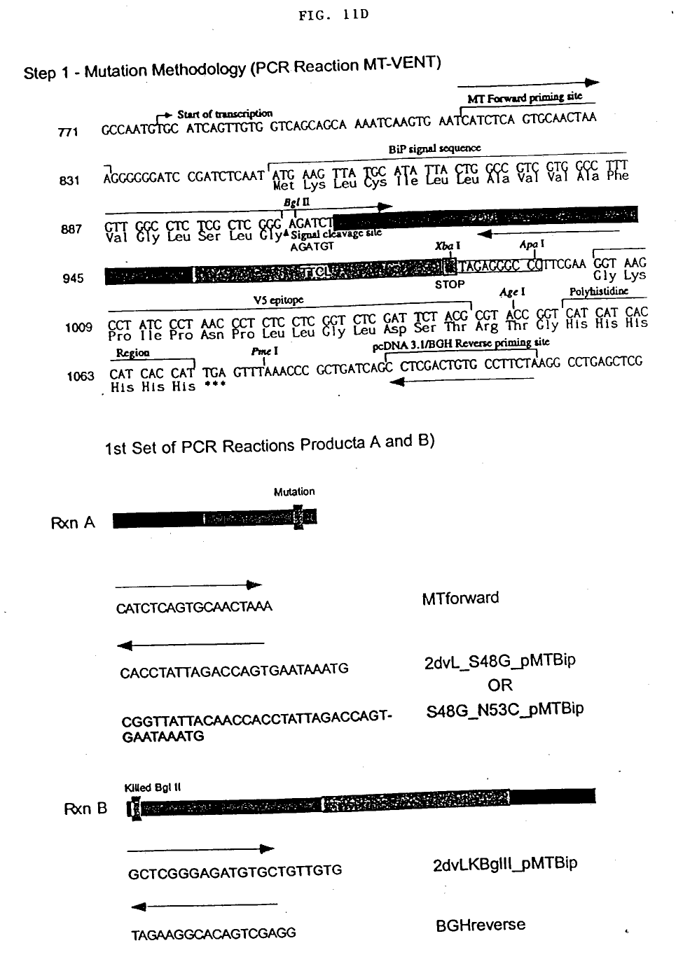

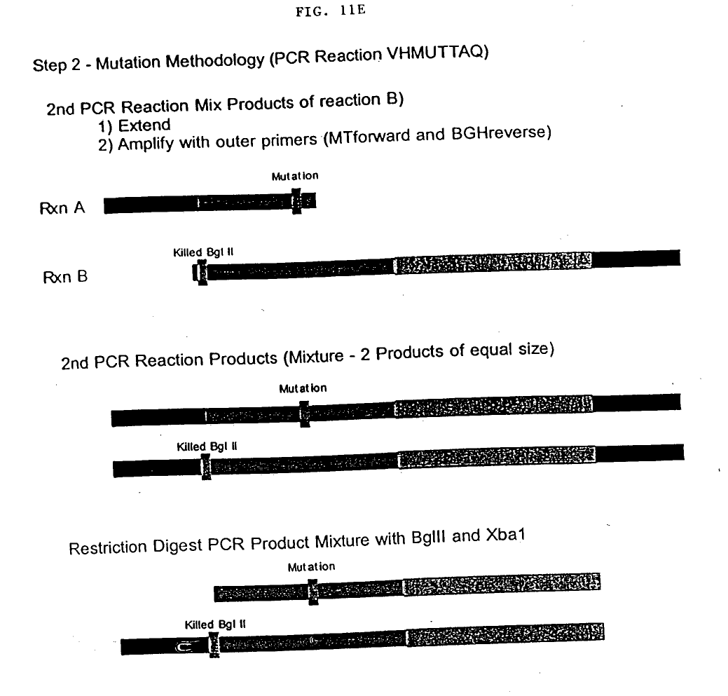

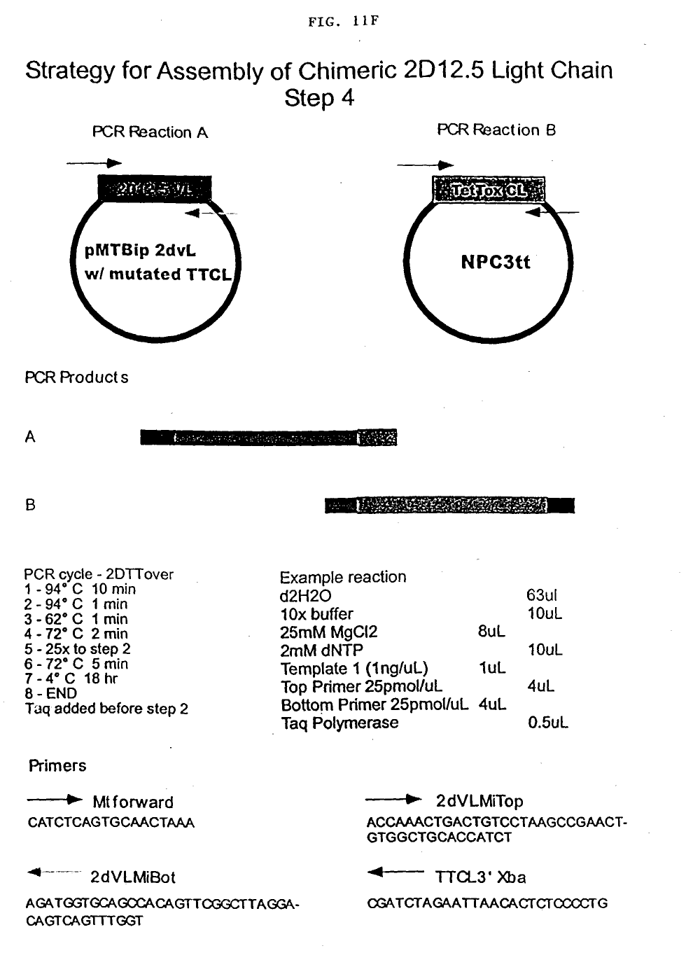

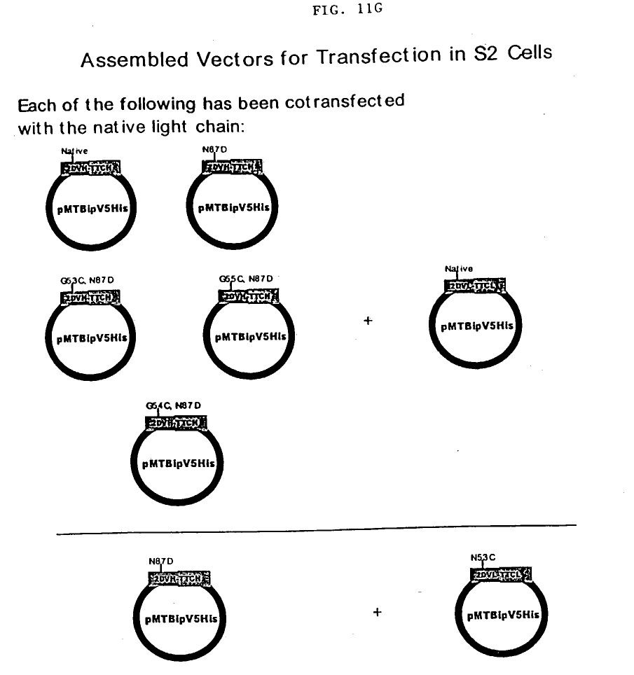

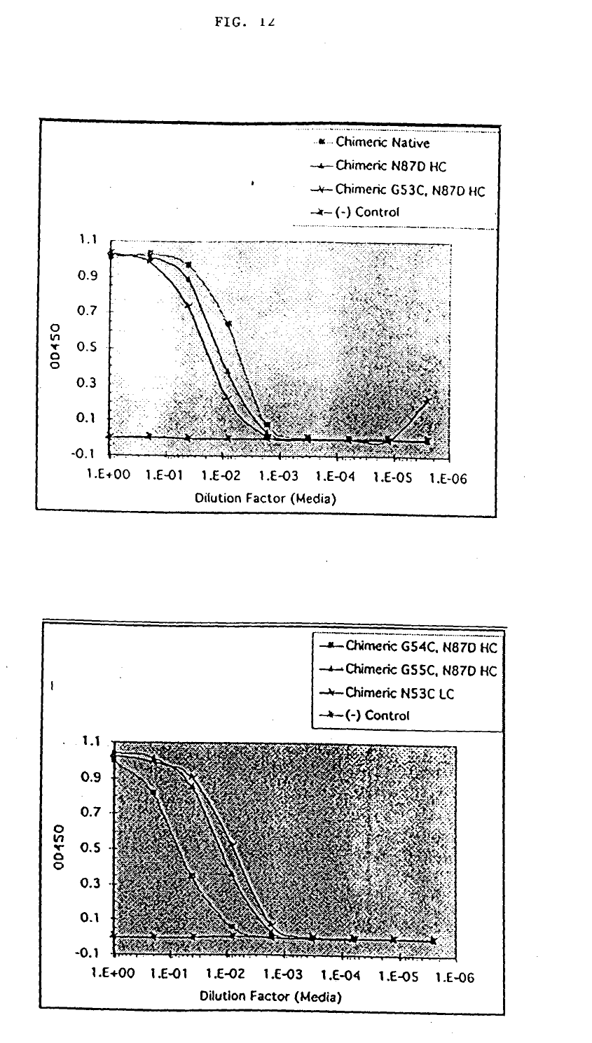

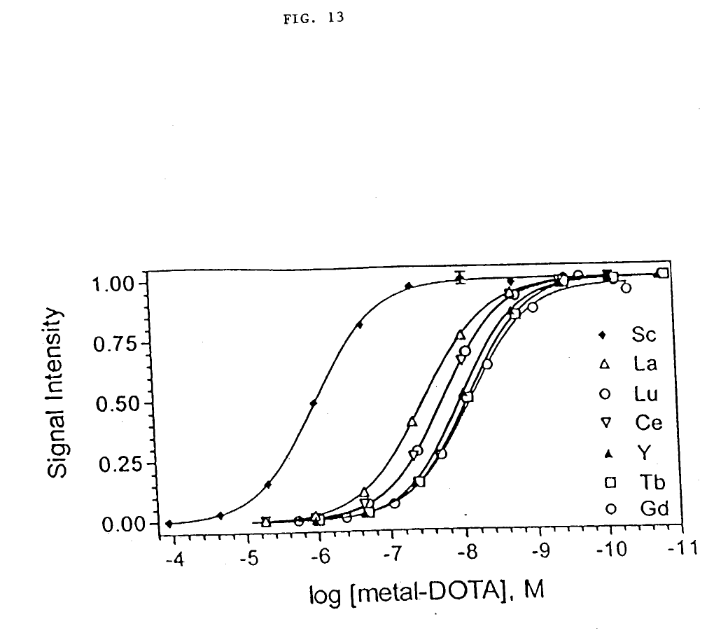

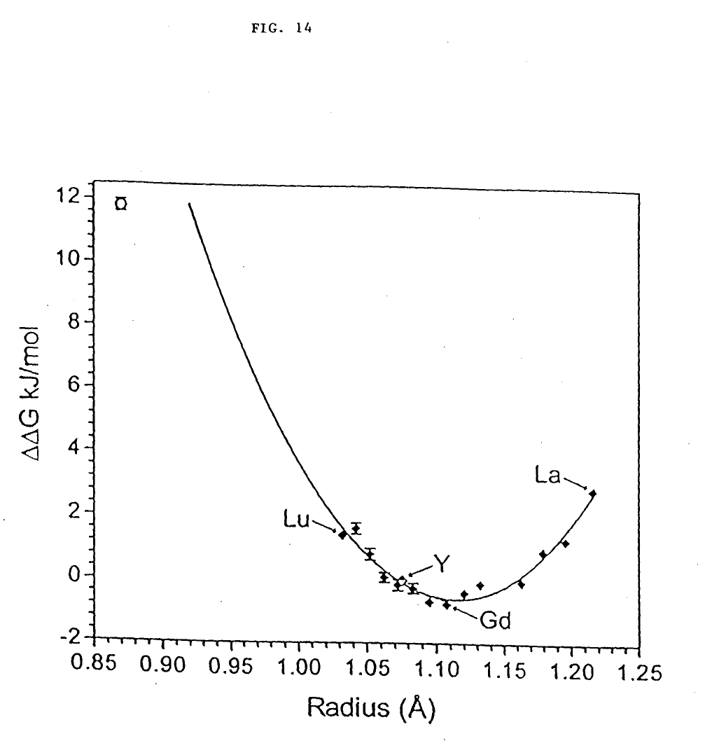

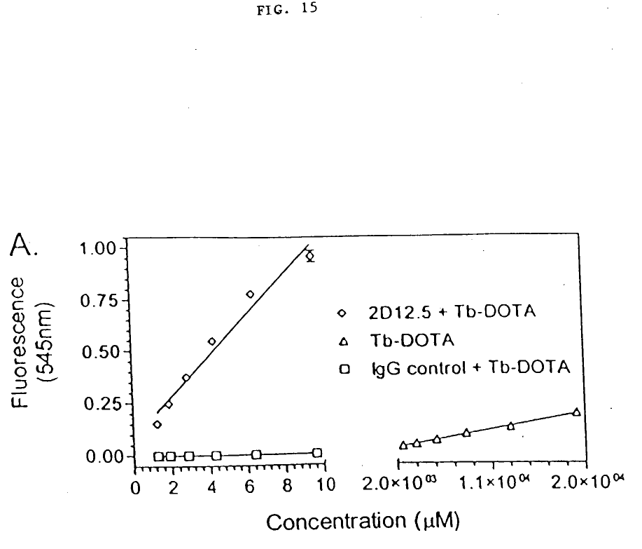

The relative binding affinities determined for each rare earth DOTA complex relative to Y-DOTA are plotted as ΔΔG values in FIG. 13. Out of 15 ions tested, we found six rare earth complexes with ΔΔG values more favorable for binding than the original Y³⁺ complex. The radii of the nonacoordinate trivalent lanthanide ions vary in small increments across the series from 1.21 Å (La³⁺) to 1.03 Å (Lu³⁺) (see, e.g., Shannon, R. D. Acta Crystallogr., Sect. A: Found. Crystallogr. A32: 751-767 (1976)). Our results show that when the shape of the DOTA complex is perturbed by either increasing or decreasing the radius of the lanthanide ion, the stability of the protein-ligand complex changes in a regular fashion. The effect of the change in ion radius on the standard ΔG of binding should be described approximately by an equation of the form

$$\frac{ⅆ Δ G}{ⅆ r} = k ( r - {r}_{0} ) , which integrates to ΔΔ G = \frac{1}{2} {k ( r - {r}_{0} )}^{2} .$$

The behavior of ΔΔG as a function of ionic radius fits a parabola, as might be expected for a system that behaves in a thermodynamically elastic way, obeying Hooke's law over a small range of perturbations. The quantitative binding differences allow us to assess the system's flexibility expressed as the force constant k, whose value is ≅50 Nm−1, comparable to a chemical bond. The optimal ionic radius r₀ predicted from the parabolic fit to the binding data is 1.11 Å, close to the strongest binders Tb³⁺, Gd³⁺, and Eu³⁺ (1.095, 1.107, and 1.120 Å).

Both Y-DOTA and Gd-DOTA are generally regarded as nonacoordinate, with 4 nitrogens and 4 oxygens from DOTA plus a single coordinated water molecule, in a capped square antiprism arrangement (denoted M). At equilibrium, rare earth DOTA complexes at either end of the lanthanide series differ in the layout of the acetate arms, and mixtures of isomers ranging from M to a distorted inverted antiprism (m) are observed for some. The ionic radius and geometry of the complex also affect the accessibility of the ninth coordination site for water (see, e.g., Aime et al., Inorg. Chem. 36: 2059-2068 (1997) and Cosentino et al., Am. Chem. Soc. 124: 4901-4909 (2002)). Large La-DOTA is almost exclusively isomer m, while small Lu-DOTA is predominantly isomer M. We suspect that these conformational equilibria play a role in the different binding affinities observed here. Even though Sc³⁺ generally exhibits similar coordination geometries to the rare earths (see, e.g., Zhang et al., Acta Crystallogr., Sect. C: Cryst. Struct. Commun. 55: 1418-1420 (1999), Sc-DOTA does not fit well on the optimal parabola for the lanthanides, perhaps because it has a much smaller ionic radius (0.867 Å) (see, e.g., Meehan et al. Chem. Rev. 181: 121-145 (1999)).

The broad specificity and high affinity of this antibody for all rare earth-DOTA complexes make it particularly interesting for applications that take advantage of the unique characteristics of lanthanides. For example, Simeonov et al. have recently described blue-fluorescent antibodies, potential sensors that change the emission of a stilbene ligand upon antibody binding (see, Simeonov, et al., Science 290: 307-313 (2000)). UV excitation of the Tb-DOTA-2D12.5 complex leads to energy transfer from aromatic side chains of the antibody to bound Tb-DOTA, enhancing green terbium luminescence by approximately four orders of magnitude relative to unbound Tb-DOTA (FIG. 3). The enhancement is comparable to that observed for Ca²⁺ binding proteins, which also transfer energy from aromatic side chains to Tb³⁺ ions bound in Ca²⁺ sites (see, e.g., Hogue et al., J. Biol. Chem. 267: 13340-13347 (1992)). Sensors based on lanthanide luminescence exhibit millisecond emission lifetimes, which makes them useful for a number of biological applications (see, e.g., Parker et al., Chem. Rev., 102: 1977-2010 (2002)).

### Example 4

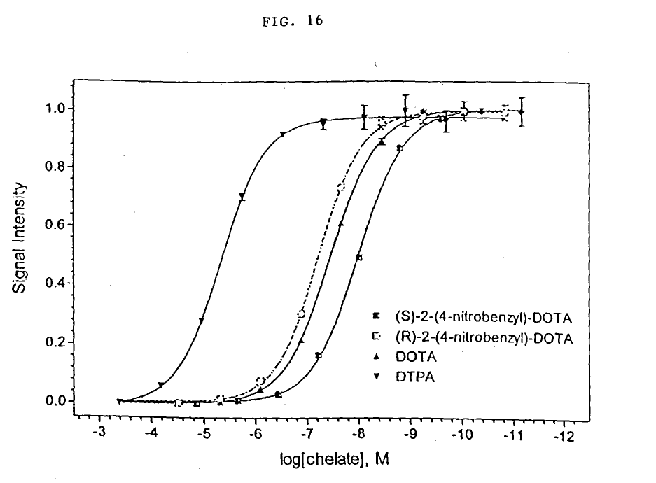

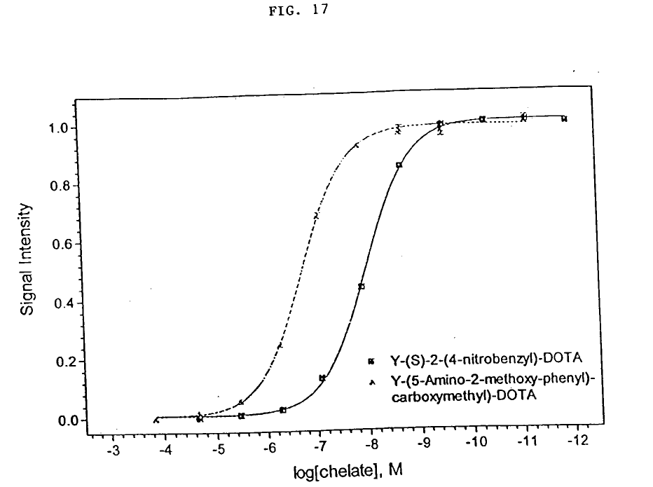

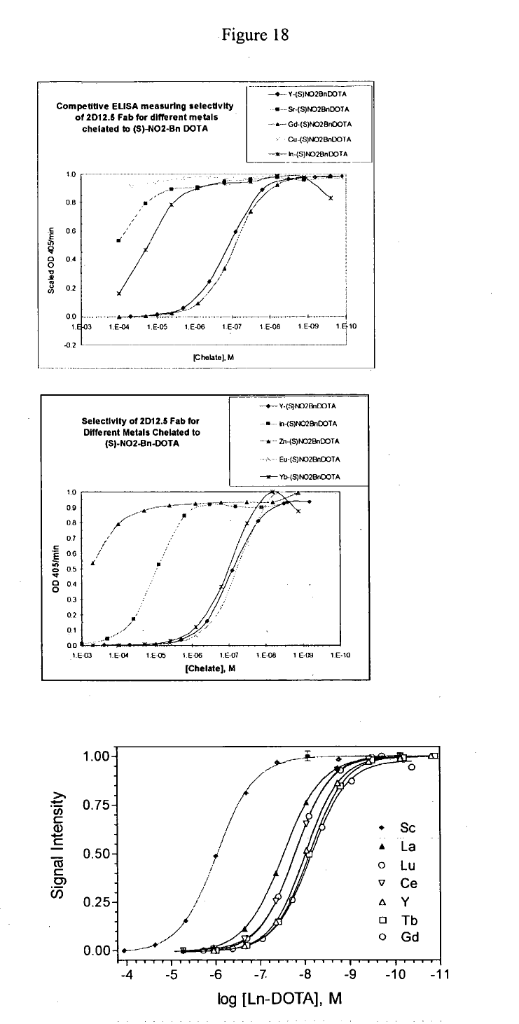

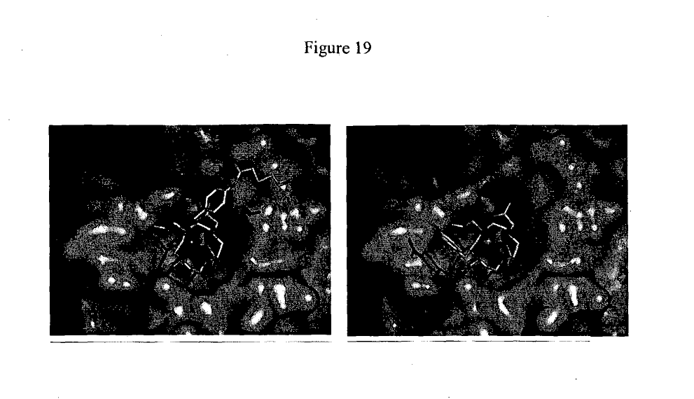

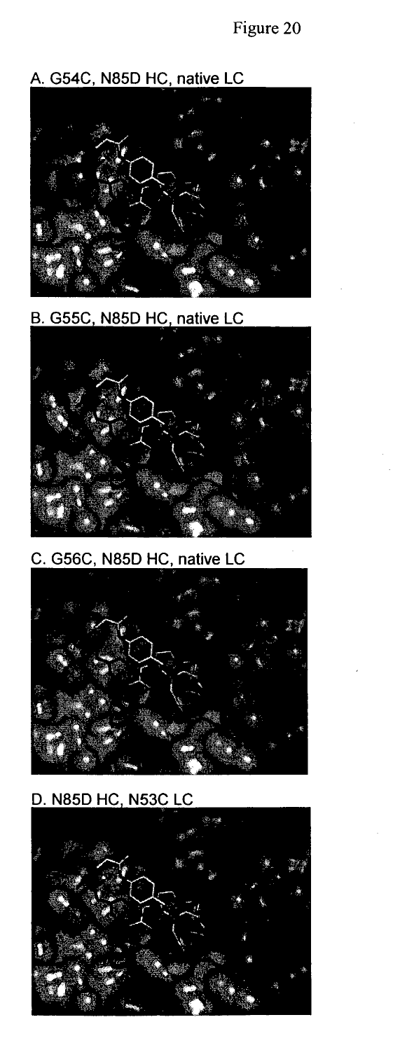

Relative binding affinities of NBD complexes of various metals ions relative to Y-NBD were determined by methods known in the art and described herein and shown in FIG. 18.

### Example 5

Materials and Methods

Chelate Synthesis. Functional derivatives of DOTA (1,4,7,10-tetraazacyclododecane-N,N′,N″,N′″-tetraacetic acid) were prepared. (S)-2-(4-aminobenzyl)-DOTA (ABD) and (S)-2-(4-(2-bromo)-acetamido)-benzyl)-DOTA (BAD) were prepared according to published methods. (S)-2-(4-acrylamidobenzyl)-DOTA (AABD), a novel electrophilic derivative of DOTA, was prepared using methods similar to those used to produce BAD and that are known to those skilled in the art. Scheme 2 depicts DOTA derivatives.

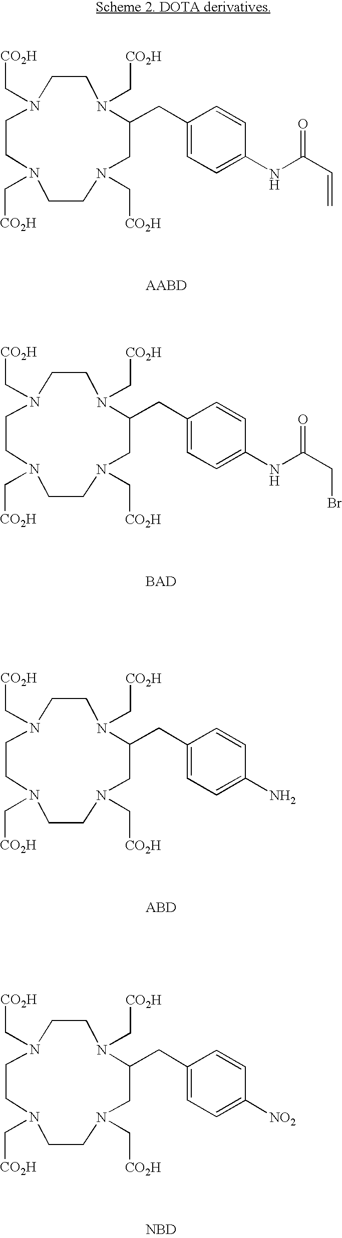

Chimeric 2D12.5 mutants. Methods for site directed mutagenesis were as described in Example 2 and shown in FIG. 10 (heavy chain) and FIG. 11 (light chain). Chimeric 2D12.5 mutants used in Examples 6-10 include heavy chain mutations, wherein aspartic acid was introduced at position 85 (N85D; numbers used in this and the following examples correspond to the Kabat numbering system) to remove native glycosylation and a cysteine residue may have been introduced at position 54, 55 or 56 (G54C, G55C, G56C, respectively). In some instances light chain mutations were made, introducing a cysteine at position 53 (N53C). The mutants used in the following examples include: N85D_G54C; N85D_G55C; N85D_G56C; N85D; and N85D, N53C (light chain).

### Example 6

This example describes a method used to purify chimeric 2D12.5 Fab molecules expressed in Schneider (S2) cells using affinity chromatography. Gd-DOTA affinity column were prepared and protocols were developed to isolate pure chimeric 2D12.5 protein from dialyzed S2 expression media. Native 2D12.5 Fab molecules as well as N85D_G54C and N85D were purified by the following method.

Isolation of chimeric 2D]2.5 Fab expressed and excreted from Schneider (S2) cells. Protein samples used in this experiment were isolated from serum free expression media used to support S2 cells. In other experiments, protein samples were isolated from media containing 10% serum. Cells were grown in suspension to an approximate density of 1×10⁷ cells/mL and induced with 500 μM CuSO₄ for 3 days. 600-800 mL of media was concentrated by tangential flow filtration (TFF) to approximately 50 mL and dialyzed into 10 mM MOPS, 100 mM NaCl, 1 mM EDTA, pH 7.2. The protein solutions were stored at 4° C.

Preparation of Gd-ABD Aminolink column for affinity purification of chimeric 2D12.5 Fab expressed in S2 cells. Approximately 10 mL of 1 mM Gd-ABD in 50 mM Na₂CO₃, 100 mM NaCl, pH 10 was added to Aminolink gel that had been pre-equilibrated in Na₂CO₃, 100 mM NaCl, pH 10 and agitated overnight at room temperature. The solution was drained and the gel was washed with several column volumes of PBS, pH 7.2. NaCNBH₃ (reducing agent) was then added to a final concentration of 100 mM, and the mixture was agitated again for several hours. The column was then washed with PBS and unreacted sites on the gel were blocked and reduced with 1 M Tris, pH 7.4 and 100 mM NaCNBH₃. The column was then washed with TBS, preservative was added, and the column was stored at 4° C.

Affinity Purification of chimeric 2D12.5 Fab expressed in S2 cells. After binding expressed chimeric 2D12.5 protein, the column was washed with a series of different solutions: high salt (e.g., ˜1 M NaCl or similar), low pH (e.g., 0.1% TFA or 0.5% acetic acid pH 2-3), high pH (e.g., 0.1% TEAOAc, ˜pH 10), and an organic additive wash containing less than or equal to 30% acetonitrile or equivalent. A neutral buffer or water was used between each of the above washes. A final wash of water was followed by elution of the chimeric 2D12.5 protein with an acidic solution containing acetonitrile or equivalent (e.g., 0.1% TFA in 20% acetonitrile). The pH of the solution was quickly adjusted to neutral and dialyzed or vacuum evaporated to remove organic solvent.

Assessing Purification. Affinity purification of chimeric 2D12.5 Fab was monitered by separation of the load, wash and elution profiles by SDS-PAGE and visualization with Sypro ruby total protein stain. Protein samples were boiled prior to separation but not reduced. 10-20% Tris-glycine SDS-PAGE gels were loaded with protein purified from media containing 10% serum or with protein purified from serum-free media. The following samples were loaded: unattached protein or flow-through, TBS wash, NaCl wash, pH 2.5 wash, pH 10 wash, pH 7, 20% acetonitrile (ACN) wash, and pH 2, 20% ACN elutions. Following separation, protein was visualized using Sypro ruby total protein stain by known methods.

The binding between antibody 2D12.5 and its high binding Metal-DOTA antigens was unaffected by varying pH alone (pH 1.5-12), high salt solutions (˜1 M) or organic solvent additive conditions (approx 30% acetonitrile or less) where the pH is neutral. This allows for a variety of wash conditions to remove contaminating untagged species that may be present in a mixture. Elution of the tagged species can be accomplished with a combination solution of low pH and an organic additive (approx 20% acetonitrile or equivalent). For example, N85D_G53C eluted in pH 2, 20% ACN buffer, as was demonstrated by the presence of a ˜50 kD band visualized by SDS-PAGE. Other elution and wash conditions are possible and will be apparent to those skilled in the art. Affinity purified mutants were used in the following examples.

### Example 7

This example demonstrates that the DOTA derivative, AABD, can form a permanent bond with the 2D12.5 mutants. AABD incorporates a weakly electrophilic acryl functionality that has been shown to be unreactive with nucleophilic species naturally present in vivo (Chmura et al., J. Controlled Release 78:249-58, 2002). Isolated and purified 2D12.5 mutants were allowed to bind to ABD, AABD or BAD complexes of Y-90 under physiologically relevant temperature and pH. BAD and ABD were used as positive and negative controls, respectively. Following a 2.5 hour incubation, samples were examined to determine whether a permanent bond formed between engineered 2D12.5 single cysteine mutants and Y-90-AABD.

Metallation of Chelates with Y-90. Y-90 was received as a solution in dilute HCl. Approximately 100 μCi (˜40 nM) of Y-90 was incubated with 0.5 mM AABD in a total volume of 50 μL 0.1M triethylammonium acetate buffer for 50 min at 70° C. TLC confirmed near-completion of the complexation reaction. The chelate was used in large excess relative to Y-90, so the sample was split in half. The free chelate was scavenged with non-radioactive Y³⁺ in one tube and non-radioactive Sr²⁺ in the other. The solutions were stored at −70° C. prior to use.

Incubation of Y-90 chelates with engineered 2D12.5 Fab proteins. The protein and metal complex solutions were estimated to be 1 μM. However, the proportion of the metal complex in each sample that included Y-90 was approximately 4-orders of magnitude less than non-radioactive metal. The metal complex solutions and protein solutions were mixed in 100 mM MOPS, 100 mM NaCl, pH 7.2 and allowed to incubate for 2.5 hours at 37° C. Samples were denatured with standard non-reducing sample application buffer (SAB) or disulfides were reduced with SAB containing 1% mercaptoethanol. Samples were boiled for 5 min, and were then applied (400 nCi per lane) to a 10-20% Tris-glycine SDS-PAGE gel and separated at 150V for 1.5 hours. Denaturation destroyed the binding sites, breaking any reversible binding interactions between the protein and ligand. Permanent bonds were not broken by the sample treatment and analysis. ABD, AABD or BAD complexes of Y-90 that were not permanently bound, migrated with the dye front at the bottom of the gel. Gels were exposed to film for 5.5 days and then developed to visualize protein bands indicating formation of a permanent bond.

Samples of the chimeric 2D12.5 mutant N85D_G54C bound to Y-AABD were detected by autoradiography as ˜50 kD (non-reduced) and ˜25 kD(reduced) bands on an SDS-PAGE gel, demonstrating the formation of a permanent bond. The affinity purified chimeric 2D12.5 mutant N85D was not detected by autoradiography, demonstrating that it only bound reversibly, as expected, since a nucleophilic amino acid was not engineered in the binding pocket. Negative controls incorporating the non-electrophilic Y-90-ABD were also not detected.

The samples of Y-90-AABD containing excess Sr-AABD displayed darker bands because Sr-AABD bound more weakly (˜0.1%) to 2D12.5 than Y-AABD. Consequently, Sr-AABD did not hinder the permanent binding between Y-90-AABD and 2D12.5. The samples of Y-90-AABD containing excess Y-AABD competed for permanent binding. Because the non-radioactive Y-AABD was in excess relative to Y-90-AABD, the 2D12.5 protein band appeared lighter. The portion of 2D12.5 proteins that bound permanently to non-radioactive Y-AABD were not visible in the SDS-PAGE gel, thereby decreasing the intensity of the bands.

### Example 8

This example demonstrates permanent binding with a collection of isolated and purified chimeric 2D12.5 Fab molecules: N85D_G54C; N85D_G55C; N85D_G56C; N85D; and N85D, N53C (light chain). 2D12.5 mutants were allowed to bind to ABD, AABD or BAD complexes of Y-90 under physiologically relevant temperature and pH. Following a 2.5 hour incubation, it was determined whether a permanent bond formed between engineered 2D12.5 single cysteine mutants and Y-90-AABD. BAD and ABD were used as positive and negative controls, respectively.

Incubation of Y-90 chelates with engineered 2D12.5 Fab proteins. The 2D12.5 solutions were estimated to be 120 nM at most. Only radioactive Y-90 complexes were used in this experiment, and were approximated to be 0.5 nM. The radioactive metal complex solutions and protein solutions were mixed and incubated as described in Example 7. Samples were denatured and reduced and separated by SDS-PAGE as described in the Example 7. Gels were exposed to a phosphor plate sensitive to radioactive decay for 20 min. The phospor plate was visualized using a phosphorimager (Molecular Dynamics Typhoon 8600) to detect protein bands indicating formation of a permanent bond.

All of the chimeric single-cysteine 2D12.5 mutants bound to Y-90-AABD and Y-90-BAD and formed a permanent bond. There were two possible orientations for the chelate in the binding pocket as shown in FIG. 1. The N85D, N53C (light chain) mutant was designed to bind permanently when the chelate is in one binding mode while the G54C, G55C and G56C mutants were designed to bind permanently when the chelate is in the other binding mode.

The affinity purified chimeric 2D12.5 mutant N85D only bound reversibly as expected. Because the sample treatment for the gel destroyed the reversible interaction, even the Y-90 complexes of AABD and BAD dissociated, leaving the protein bands in those particular lanes non-radioactive. Negative controls incorporating the non-electrophilic Y-90-ABD were also non-radioactive, as expected.

### Example 9

This example describes the rates of formation of a permanent bond between Y-90-AABD and a collection of isolated and purified chimeric 2D12.5 Fab molecules: N85D_G54C; N85D_G55C; N85D_G56C; N85D; and N85D, N53C (light chain). From the above molecules, the fastest permanent bond forming single-cysteine mutant was determined and estimates of permanent bond formation rates for the various single-cysteine mutants were obtained. 2D12.5 Fab mutants were incubated with Y-90 chelates as described in EXAMPLE 7.

Each single-cysteine mutant sample was a large volume sample. The zero-time point was protein before Y-90-AABD was added. Aliquots were then withdrawn at the indicated time points (ranging from 1 minute to 18 hours) and immediately denatured and reduced with reducing sample application buffer (SAB) containing 1% mercaptoethanol. Samples were boiled for 5 min, and flash frozen in liquid N2 before storing at −20° C. Samples were then separated by SDS-PAGE, as previously described. Gels were exposed to a phosphor plate sensitive to radioactive decay for 60 min to visualize protein bands indicating formation of a permanent bond.

All of the chimeric 2D12.5 mutants were detected by phosphor imaging, indicating all bound to Y-90-AABD and formed a permanent bond. These experiments indicated that the G54C and G56C mutations formed permanent bonds with the Y-90-AABD ligand most quickly followed by the G55C mutant. The N53C (light chain) mutant had the weakest activity for formation of a permanent with Y-90-AABD. The affinity purified chimeric 2D12.5 mutant N85D only bound reversibly, as expected. Negative controls incorporating the non-electrophilic Y-90-ABD were also non-radioactive, as expected.

### Example 10

This example demonstrates the permanency of rare earth-AABD complex binding to the N85D_G54C 2D12.5 mutant and approximates the lower limits of reversible affinity required for formation of a permanent bond. The native antibody 2D12.5 binds all rare earth DOTA complexes with high affinity (Corneillie et al., J. Am. Chem. Soc. 125:3436-37, 2003) and other metals with affinities that are weaker. Permanent binding between four single-cysteine mutants of 2D12.5 and Y-90-AABD has been demonstrated. Example 9 indicated that the N85D_G54C mutant reacted most quickly with Y-90-AABD. Therefore, this mutant was selected for the present example.

Metallation of Chelates with stable metal cations. Metals tested in this experiment included Y, La, Lu, Sr and Sc. Stock chelate concentrations were measured using a Co-57 metal binding assay (Meares et al., Anal. Biochem. 142:68-78, 1984). Stock metal solutions (approximately 0.2 M) were prepared by dissolving the appropriate gravimetrically measured rare earth chloride salt in 0.05 M HCl. As an example, YCl₃ (2.5 equivalents) was added to 25 μL of NBD (24.5mmol) dissolved in 0.1 M tetramethylammonium acetate, and the pH was adjusted to 5 by the addition of triethylamine. The metallation was allowed to proceed overnight at 50° C. Samples were promptly frozen at −70° C., and metallation efficiency was assessed using a competitive metal-binding assay with Co-57³. Excess metal was scavenged with DTPA, which has very low affinity for the antibody (<<1%).

Metal-AABD and metal-NBD complexes were incubated with 2D12.5 Fab mutant N85D_G54C and samples denatured, reduced and separated by SDS-PAGE, as described for Y-90 in the previous examples. Proteins were transferred to a PVDF membrane, and the Western blot was stained using hybridoma-derived 2D12.5 as the 1° stain and a goat anti-mouse lambda-horse radish peroxidase conjugate (HRP) (specific for the light chain of hybridoma-derived 2D12.5, chimeric expressed Fab light chains are a human kappa subtype and are not stained with the 2° antibody). The blot was then visualized using a chemifluorescent substrate and an imager sensitive to this type of substrate (Molecular Dynamics Storm 860).

N85D_G54C 2D12.5 was detected on the western blots for samples containing Y, La, Lu and Sc-AABD complexes. This indicated that N85D_G54C 2D12.5 mutant bound permanently to all AABD complexes of the rare earths (inferred from the permanent binding data with AABD complexes of Y, La and Lu). Western blots demonstrated NBD complexes of La³⁺ and Lu³⁺ to be the weakest binders of the rare earths, while Y³⁺ was to be one of the strongest binders. It can be predicted from this data that all AABD complexes of the rare earths will bind permanently to the appropriate 2D12.5 single cys-mutants. Sc-AABD was the experimentally determined lower limit for successful permanent bond formation. The AABD complex of Sr²⁺ was unsuccessful in forming a permanent bond with the test single-cysteine mutant. Its measured affinity (Sr-NBD) was approximately one order of magnitude weaker than Sc-NBD.

The method used to detect permanent bond formation relied on hybridoma-derived 2D12.5 as the 1° stain of the Western blot. This interaction was reversible and was considerably weaker for Sc-AABD and Sr-AABD (˜1-order weaker than for Sc-AABD) than for AABD complexes of the rare earths. The fact that Sc-AABD permanent bond formation with the N85D_G54C 2D12.5 mutant was visible indicated that the extent of permanent bond formation was likely to be much higher than was observable in this experiment, because the intensity of the band was dependent on the amount of hybridoma-derived 2D 12.5 bound to the protein band on the blot. Weaker reversible binders may, in fact, bind permanently as well.

The results of this experiment show how permanent binding might expand the use of a particular system. In particular, a weak, reversibly binding protein-ligand pair, may not be very useful experimentally; however, if that weak affinity is strong enough, that weak, reversibly binding protein-ligand pair can be transformed into a permanent binding system, surpassing the affinity of the commonly used streptavidin-biotin binding pair. This is exemplified with the Sc-AABD-2D12.5 (N85D_G54C, N53C (light chain)) permanent binding system. Negative controls incorporating the non-electrophilic NBD were negative as expected.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

1. A mutant antibody comprising a reactive site not present in the wildtype of said antibody and an antigen recognition domain that recognizes a macrocyclic metal chelate comprising four nitrogen atoms, wherein said reactive site is in a position proximate to or within said antigen recognition domain.

2. The mutant antibody of claim 1, wherein at least two of said nitrogen atoms are covalently linked to a substituted or unsubstituted ethyl bridge.

3. The mutant antibody of claim 2, wherein said metal chelate comprises the subunit: 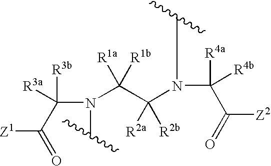
wherein
Z¹ and Z² are members independently selected from OR and NR³R⁴ in which
R³ and R⁴ are members independently selected from H, substituted or unsubstituted alkyl and substituted or unsubstituted heteroalkyl;
R^{1a}, R^{1b}, R^{2a}, R^{2b}, R^{3a}, R^{3b}, R^{4a} and R^{4b} are members independently selected from H, substituted or unsubstituted alkyl, substituted or unsubstituted heteroalkyl, substituted or unsubstituted aryl and linker moieties.

4. The mutant antibody of claim 1, wherein said chelate is a member selected from substituted or unsubstituted DOTA and substituted or unsubstituted TETA.

5. The mutant antibody of claim 4, wherein said chelate has the formula: 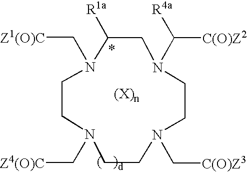
wherein
Z¹, Z², Z³ and Z⁴ are members independently selected from OR¹ and NR¹R² in which
R¹ and R² are members independently selected from H, substituted or unsubstituted alkyl and substituted or unsubstituted heteroalkyl;
X is a member selected from a lanthanide, an actinide, an alkaline earth metal, a group IIIb transition metal, or a metal;
n is 0 or 1; and
d is 1 or 2.

6. The mutant antibody of claim 1, wherein said macrocyclic metal chelate comprises a reactive functional group having reactivity complementary to said reactive site of said mutant antibody.

7. The mutant antibody of claim 5, wherein the carbon atom marked * is of S configuration.

8. The mutant antibody of claim 1, comprising a moiety having the formula: 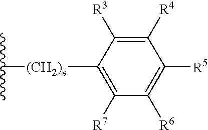
wherein
R³, R⁴, R⁵, R⁶ and R⁷ are members independently selected from H, halogen, NO₂, CN, X¹R⁸, NR⁹R¹⁰, and C(X²)R¹¹
wherein
X¹ is a member selected from O, NH and S;
R⁸ and R⁹ are members independently selected from H, substituted or unsubstituted alkyl, substituted or unsubstituted heteroalkyl and C(Z³)R¹²
wherein
X³ is a member selected from O, S and NH;
R¹² is a member selected from substituted or unsubstituted alkyl, substituted or unsubstituted heteroalkyl and OR¹³
wherein
R¹³ is a member selected from substituted or unsubstituted alkyl, substituted or unsubstituted heteroalkyl, substituted or unsubstituted aryl and substituted or unsubstituted heteroaryl;
R¹⁰ is a member selected from H, substituted or unsubstituted alkyl, substituted or unsubstituted heteroalkyl and OH,
and R⁹ and R¹⁰, taken together are optionally (═C═S);
X²is a member selected from O, S and NH; and
R¹¹ is a member selected from H, halogen, substituted or unsubstituted alkyl, substituted or unsubstituted heteroalkyl, OR¹⁴, NR¹⁵R¹⁶
wherein
R¹⁴ is a member selected from H, substituted or unsubstituted alkyl, substituted or unsubstituted heteroalkyl, and C(O)R¹⁷
wherein
R¹⁷ is a member selected from substituted or unsubstituted alkyl and substituted or unsubstituted heteroalkyl; and
R¹⁵ and R¹⁶ are members independently selected from H, substituted or unsubstituted alkyl and substituted or unsubstituted heteroalkyl.

9. The mutant antibody according to claim 8, wherein R¹⁰ is —C(O)—CHCH₂.

10. The mutant antibody according to claim 1, wherein said reactive site is a side-chain of a naturally occurring or non-naturally occurring amino acid.

11. The mutant antibody according to claim 10, wherein said reactive site is the —SH group of cysteine.

12. The mutant antibody according to claim 10, further comprising said metal chelate bound to said antigen recognition domain, wherein said metal chelate comprises a reactive functional group of complementary reactivity to said reactive site of said antibody.

13. The mutant antibody according to claim 12, further comprising a covalent bond between reactive site of said antibody and said reactive functional group of said metal chelate.

14. The mutant antibody according to claim 1, wherein said mutant antibody is mutant of 2D12.5.

15. The mutant antibody according to claim 14, wherein asparagine-88 residue of the heavy-chain is substituted by an aspartic acid residue.

16. The mutant antibody according to claim 15, wherein glycine-54 residue of the heavy-chain is substituted by a cysteine residue.

17. The mutant antibody according to claim 15, wherein glycine-55 residue of the heavy-chain is substituted by a cysteine residue.

18. The mutant antibody according to claim 15, wherein glycine-56 residue of the heavy-chain is substituted by a cysteine residue

19. The mutant antibody according to claim 14, wherein asparagine-54 of the light chain is substituted by a cysteine residue.

20. The mutant antibody of claim 1, further comprising a targeting moiety.

21. The mutant antibody according to claim 20, wherein said targeting moiety binds specifically to a cell surface protein.

22. The mutant antibody according to claim 20, wherein the targeting moiety is covalently attached to said mutant antibody.

23. The mutant antibody according to claim 21, wherein the targeting moiety is an antibody.

24. The mutant antibody according to claim 22, wherein the targeting moiety specifically binds to a protein on a cancer cell.

25. A composition having the structure:
(Ab)_{n′}-L-T
wherein,
n′ is an integer from 1-10;
Ab represents a mutant antibody according to claim 1;
L is a chemical bond or linking group that may contain one or more functional groups; and
T is said targeting moiety.

26. The composition of claim 25, wherein said targeting moiety is an antibody that binds specifically to a cell surface antigen.

27. A pharmaceutical composition comprising the composition of claim 25, and a pharmaceutically acceptable carrier.

28. A method of in vivo imaging, said method comprising the steps of:
(a) administering to said subject a mutant antibody of claim 1, wherein said antibody comprises a targeting moiety that binds specifically to a cell by binding with a member selected from the group consisting of cell surface receptors and cell surface antigens, thereby forming a cell-mutant antibody complex; and
(b) administering to said subject said metal chelate, thereby specifically binding said compound to said mutant antibody to form a cell-antibody-metal chelate complex; and
(c) detecting said cell-mutant antibody-metal chelate complex.

29. The method of claim 28, wherein the step of detecting is by positron emission tomography.

30. The method of claim 28, wherein the step of detecting is by magnetic resonance imaging.

31. The method of claim 28, wherein the step of detecting is by detection of lanthanide luminescence.

32. The method of claim 28, further comprising, between steps (a) and (b), administering a clearing agent to said subject.

33. The method of claim 28, wherein the subject is a mammal.

34. The method of claim 33, wherein the mammal is a human.

35. A method of treating a subject with cancer by administration of a metal chelate, said method comprising the steps of:
(a) administering to said subject a mutant antibody of claim 1, wherein said antibody comprises a targeting moiety that binds specifically to a cell by binding with a member selected from the group consisting of cell surface receptors and cell surface antigens, thereby forming a cell-mutant antibody complex; and
(b) administering to said subject said metal chelate, thereby specifically binding said compound to mutant antibody to form a cell-mutant antibody-metal chelate complex.

36. The method of claim 35, wherein the subject is a mammal.

37. The method of claim 36,.wherein the mammal is a human.

38. An isolated nucleic acid, comprising a sequence selected from a sequence as set forth in SEQ ID NOS:16, 17, 18, 19, 20, 25, 26, 31, 32, 42, 43, 44, 45, 46, and 47.

39. A polypeptide comprising an amino acid sequence selected from a sequence as set forth in SEQ ID NOS: 10, 11, 12, 13, 14, 22, 23, 27, 28, 35, 36, 37, 38, 39, and 40.

40. The isolated nucleic acid according to claim 38, further comprising a promoter operably linked to the nucleic acid sequence.

41. An expression vector comprising the nucleic acid according to claim 40.

42. A host cell comprising the expression vector according to claim 41.


SEQUENCE LISTING (72 sequences)

SEQ ID NO: 1 - protein, 110 aa, Mus musculus
  murine monoclonal antibody 2D12.5 light chain variable domain (VL)
QAVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTRLTVLG

SEQ ID NO: 2 - protein, 14 aa, Mus musculus
  murine monoclonal antibody 2D12.5 light chain variable domain (VL) complementarity determining region 1 (CDR1)
RSSTGAVTTSNYAN

SEQ ID NO: 3 - protein, 7 aa, Mus musculus
  murine monoclonal antibody 2D12.5 light chain variable domain (VL) complementarity determining region 2 (CDR2)
GNNNRPP

SEQ ID NO: 4 - protein, 9 aa, Mus musculus
  murine monoclonal antibody 2D12.5 light chain variable domain (VL) complementarity determining region 3 (CDR3)
ALWYSNHWV

SEQ ID NO: 5 - protein, 119 aa, Mus musculus
  murine monoclonal antibody 2D12.5 heavy chain variable domain (VH)
QVKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSS

SEQ ID NO: 6 - protein, 5 aa, Mus musculus
  murine monoclonal antibody 2D12.5 heavy chain variable domain (VH) complementarity determining region 1 (CDR1)
DYGVH

SEQ ID NO: 7 - protein, 16 aa, Mus musculus
  murine monoclonal antibody 2D12.5 heavy chain variable domain (VH) complementarity determining region 2 (CDR2)
VIWSGGGTAYTAAFIS

SEQ ID NO: 8 - protein, 11 aa, Mus musculus
  murine monoclonal antibody 2D12.5 heavy chain variable domain (VH) complementarity determining region 3 (CDR3)
RGSYPYNYFDV

SEQ ID NO: 9 - protein, 118 aa, Mus musculus
  native hybridoma murine 2D12.5 heavy chain variable region (VH)
VKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSS

SEQ ID NO: 10 - protein, 118 aa, Artificial Sequence
  Description of Artificial Sequencenative cloned hybridoma murine 2D12.5 heavy chain variable region (VH)
VKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSA

SEQ ID NO: 11 - protein, 118 aa, Artificial Sequence
  Description of Artificial SequenceN87D cloned mutant murine 2D12.5 heavy chain variable region (VH)
VKLQESGPGLVQPSQSLSITCTVSGFSLTDYGVHWVRQSPGKGLEWLGVIWSGGGTAYTAAFISRLNIYKDNSKNQVFFEMNSLQADDTAMYYCARRGSYPYNYFDVWGQGTTVTVSA

SEQ ID NO: 12 - protein, 118 aa, Artificial Sequence
  Description of Artificial SequenceN87D G53C cloned mutant murine 2D12.5 heavy chain variable region (VH)
VKLQESGPGLVQPSQSLSITCTVSGFSLTDYGVHWVRQSPGKGLEWLGVIWSCGGTAYTAAFISRLNIYKDNSKNQVFFEMNSLQADDTAMYYCARRGSYPYNYFDVWGQGTTVTVSA

SEQ ID NO: 13 - protein, 118 aa, Artificial Sequence
  Description of Artificial SequenceN87D G54C cloned mutant murine 2D12.5 heavy chain variable region (VH)
VKLQESGPGLVQPSQSLSITCTVSGFSLTDYGVHWVRQSPGKGLEWLGVIWSGCGTAYTAAFISRLNIYKDNSKNQVFFEMNSLQADDTAMYYCARRGSYPYNYFDVWGQGTTVTVSA

SEQ ID NO: 14 - protein, 118 aa, Artificial Sequence
  Description of Artificial SequenceN87D G55C cloned mutant murine 2D12.5 heavy chain variable region (VH)
VKLQESGPGLVQPSQSLSITCTVSGFSLTDYGVHWVRQSPGKGLEWLGVIWSGGCTAYTAAFISRLNIYKDNSKNQVFFEMNSLQADDTAMYYCARRGSYPYNYFDVWGQGTTVTVSA

SEQ ID NO: 15 - DNA, 354 bp, Mus musculus
  native hybridoma murine 2D12.5 heavy chain variable region (VH)
gtgaagctgcaggagtcaggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctaatgacacagccatgtattactgtgccagaaggggtagctacccttacaactacttcgatgtctggggccaagggaccacagtcaccgtctcctca

SEQ ID NO: 16 - DNA, 354 bp, Artificial Sequence
  Description of Artificial Sequencenative cloned hybridoma murine 2D12.5 heavy chain variable region (VH)
gtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctaatgacacagccatgtattactgtgccagaaggggtagctacccttacaactacttcgatgtctggggccaagggaccacggtcaccgtctccgca

SEQ ID NO: 17 - DNA, 354 bp, Artificial Sequence
  Description of Artificial SequenceN87D cloned mutant murine 2D12.5 heavy chain variable region (VH)
gtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgca

SEQ ID NO: 18 - DNA, 354 bp, Artificial Sequence
  Description of Artificial SequenceN87D G53C cloned mutant murine 2D12.5 heavy chain variable region (VH)
gtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagttgtggaggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgca

SEQ ID NO: 19 - DNA, 354 bp, Artificial Sequence
  Description of Artificial SequenceN87D G54C cloned mutant murine 2D12.5 heavy chain variable region (VH)
gtgaagctgcaggagtctgggcctggcctagtgcagccctcacagagcctgtccatcacctgcacggtctctggtttctcattaactgactatggtgtacactgggttcgccagtctccaggaaagggtctggaatggctgggagtgatatggagtggttgtggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgca

SEQ ID NO: 20 - DNA, 354 bp, Artificial Sequence
  Description of Artificial SequenceN87D G55C cloned mutant murine 2D12.5 heavy chain variable region (VH)
gtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggatgcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgca

SEQ ID NO: 21 - protein, 109 aa, Mus musculus
  native hybridoma murine 2D12.5 light chain variable region (VL)
AVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTRLTVLG

SEQ ID NO: 22 - protein, 109 aa, Artificial Sequence
  Description of Artificial Sequencenative cloned hybridoma murine 2D12.5 light chain variable region (VL)
AVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTKLTVLS

SEQ ID NO: 23 - protein, 109 aa, Artificial Sequence
  Description of Artificial SequenceN53C cloned mutant murine 2D12.5 light chain variable region (VL)
AVVTQESALTTSPGETVTLTCRSSTGAVTTSNYANWVQEKPDHLFTGLIGGCNNRPPGVPARFSGSLIGDKAALTIAGTQTEDEAIYFCALWYSNHWVFGGGTKLTVLS

SEQ ID NO: 24 - DNA, 327 bp, Mus musculus
  native hybridoma murine 2D12.5 light chain variable region (VL)
gctgttgtgactcaggaatctgcactcaccacatcacctggtgaaacagtcacactcacttgtcgctcaagtactggggctgttacgactagtaactatgccaactgggtccaagagaaaccagatcatttattcactggtctaataggtggtaataataaccgacctccaggtgttcctgccagattctcaggctccctgattggagacaaggctgccctcaccatcgcagggacacagactgaggatgaggcaatatatttctgtgctctatggtacagcaaccattgggtgttcggtggaggaaccagactgactgtcctaggc

SEQ ID NO: 25 - DNA, 327 bp, Artificial Sequence
  Description of Artificial Sequencenative cloned hybridoma murine 2D12.5 light chain variable region (VL)
gctgttgtgactcaggaatctgcactcaccacatcacctggtgaaacagtcacactcacttgtcgctcaagtactggggctgttacgactagtaactatgccaactgggtccaagagaaaccagatcatttattcactggtctaataggtggtaataataaccgacctccaggtgttcctgccagattctcaggctccctgattggagacaaggctgccctcaccatcgcagggacacagactgaggatgaggcaatatatttctgtgctctatggtacagcaaccattgggtgttcggtgggggaaccaaactgactgtcctaagc

SEQ ID NO: 26 - DNA, 327 bp, Artificial Sequence
  Description of Artificial SequenceN53C cloned mutant murine 2D12.5 light chain variable region (VL)
gctgttgtgactcaggaatctgcactcaccacatcacctggtgaaacagtcacactcacttgtcgctcaagtactggggctgttacgactagtaactatgccaactgggtccaagagaaaccagatcatttattcactggtctaataggtggttgtaataaccgacctccaggtgttcctgccagattctcaggctccctgattggagacaaggctgccctcaccatcgcagggacacagactgaggatgaggcaatatatttctgtgctctatggtacagcaaccattgggtgttcggtgggggaaccaaactgactgtcctaagc

SEQ ID NO: 27 - protein, 218 aa, Artificial Sequence
  Description of Artificial Sequencenative cloned chimeric murine 2D12.5 light chain variable region (VL) fused to human anti-tetanus toxin antibody kappa light chain constant region (TTCL)
RSAVVTQESALTTSPGETVTLTCRSSTGAVTTSNYANWVQEKPDHLFTGLIGGNNNRPPGVPARFSGSLIGDKAALTIAGTQTEDEAIYFCALWYSNHWVFGGGTKLTVLSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 28 - protein, 218 aa, Artificial Sequence
  Description of Artificial SequenceN53C cloned mutant chimeric murine 2D12.5 light chain variable region (VL) fused to human anti-tetanus toxin antibody kappa light chain constant region (TTCL)
RSAVVTQESALTTSPGETVTLTCRSSTGAVTTSNYANWVQEKPDHLFTGLIGGCNNRPPGVPARFSGSLIGDKAALTIAGTQTEDEAIYFCALWYSNHWVFGGGTKLTVLSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 29 - protein, 109 aa, Mus musculus; identical to SEQ ID NO: 21
  native hybridoma murine 2D12.5 light chain variable region (VL)

SEQ ID NO: 30 - protein, 107 aa, Homo sapiens
  human anti-tetanus toxin antibody kappa light chain constant region (TTCL) template
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 31 - DNA, 664 bp, Artificial Sequence
  Description of Artificial Sequencenative cloned chimeric murine 2D12.5 light chain variable region (VL) fused to human anti-tetanus toxin antibody kappa light chain constant region (TTCL)
agatctgctgttgtgactcaggaatctgcactcaccacatcacctggtgaaacagtcacactcacttgtcgctcaagtactggggctgttacgactagtaactatgccaactgggtccaagagaaaccagatcatttattcactggtctaataggtggtaataataaccgacctccaggtgttcctgccagattctcaggctccctgattggagacaaggctgccctcaccatcgcagggacacagactgaggatgaggcaatatatttctgtgctctatggtacagcaaccattgggtgttcggtgggggaaccaaactgactgtcctaagccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttaattctaga

SEQ ID NO: 32 - DNA, 664 bp, Artificial Sequence
  Description of Artificial SequenceN53C cloned mutant chimeric murine 2D12.5 light chain variable region (VL) fused to human anti-tetanus toxin antibody kappa light chain constant region (TTCL)
agatctgctgttgtgactcaggaatctgcactcaccacatcacctggtgaaacagtcacactcacttgtcgctcaagtactggggctgttacgactagtaactatgccaactgggtccaagagaaaccagatcatttattcactggtctaataggtggttgtaataaccgacctccaggtgttcctgccagattctcaggctccctgattggagacaaggctgccctcaccatcgcagggacacagactgaggatgaggcaatatatttctgtgctctatggtacagcaaccattgggtgttcggtgggggaaccaaactgactgtcctaagccgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggtctgagcttgcccgtcacaaagagcttcaacaggggagagtgttaattctaga

SEQ ID NO: 33 - DNA, 327 bp, Mus musculus; identical to SEQ ID NO: 24
  native hybridoma murine 2D12.5 light chain variable region (VL)

SEQ ID NO: 34 - DNA, 322 bp, Homo sapiens
  human anti-tetanus toxin antibody kappa light chain constant region (TTCL) template
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgtt

SEQ ID NO: 35 - protein, 227 aa, Artificial Sequence
  Description of Artificial Sequencenative cloned chimeric murine 2D12.5 heavy chain variable region (VH) fused to human anti-tetanus toxin antibody CH1 heavy chain constant region (TTCH)
RSVKLQESGPGLVQPSQSLSITCTVSGFSLTDYGVHWVRQSPGKGLEWLGVIWSGGGTAYTAAFISRLNIYKDNSKNQVFFEMNSLQANDTAMYYCARRGSYPYNYFDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKSR

SEQ ID NO: 36 - protein, 227 aa, Artificial Sequence
  Description of Artificial SequenceN87D cloned mutant chimeric murine 2D12.5 heavy chain variable region (VH) fused to human anti-tetanus toxin antibody CH1 heavy chain constant region (TTCH)
RSVKLQESGPGLVQPSQSLSITCTVSGFSLTDYGVHWVRQSPGKGLEWLGVIWSGGGTAYTAAFISRLNIYKDNSKNQVFFEMNSLQADDTAMYYCARRGSYPYNYFDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKSR

SEQ ID NO: 37 - protein, 227 aa, Artificial Sequence
  Description of Artificial SequenceN87D G53C cloned mutant chimeric murine 2D12.5 heavy chain variable region (VH) fused to human anti-tetanus toxin antibody CH1 heavy chain constant region (TTCH)
RSVKLQESGPGLVQPSQSLSITCTVSGFSLTDYGVHWVRQSPGKGLEWLGVIWSCGGTAYTAAFISRLNIYKDNSKNQVFFEMNSLQADDTAMYYCARRGSYPYNYFDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKSR

SEQ ID NO: 38 - protein, 227 aa, Artificial Sequence
  Description of Artificial SequenceN87D G54C cloned mutant chimeric murine 2D12.5 heavy chain variable region (VH) fused to human anti-tetanus toxin antibody CH1 heavy chain constant region (TTCH)
RSVKLQESGPGLVQPSQSLSITCTVSGFSLTDYGVHWVRQSPGKGLEWLGVIWSGCGTAYTAAFISRLNIYKDNSKNQVFFEMNSLQADDTAMYYCARRGSYPYNYFDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKSR

SEQ ID NO: 39 - protein, 227 aa, Artificial Sequence
  Description of Artificial SequenceN87D G55C cloned mutant chimeric murine 2D12.5 heavy chain variable region (VH) fused to human anti-tetanus toxin antibody CH1 heavy chain constant region (TTCH)
RSVKLQESGPGLVQPSQSLSITCTVSGFSLTDYGVHWVRQSPGKGLEWLGVIWSGGCTAYTAAFISRLNIYKDNSKNQVFFEMNSLQADDTAMYYCARRGSYPYNYFDVWGQGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKSR

SEQ ID NO: 40 - protein, 227 aa, Artificial Sequence; identical to SEQ ID NO: 35
  Description of Artificial Sequencechimeric murine 2D12.5 heavy chain variable region (VH) fused to human anti-tetanus toxin antibody CH1 heavy chain constant region (TTCH) expected sequence

SEQ ID NO: 41 - protein, 118 aa, Mus musculus; identical to SEQ ID NO: 9
  native hybridoma murine 2D12.5 heavy chain variable region (VH)

SEQ ID NO: 42 - DNA, 681 bp, Artificial Sequence
  Description of Artificial Sequencenative cloned chimeric murine 2D12.5 heavy chain variable region (VH) fused to human anti-tetanus toxin antibody CH1 heavy chain constant region (TTCH)
agatctgtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctaatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 43 - DNA, 681 bp, Artificial Sequence
  Description of Artificial SequenceN87D cloned mutant chimeric murine 2D12.5 heavy chain variable region (VH) fused to human anti-tetanus toxin antibody CH1 heavy chain constant region (TTCH)
agatctgtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 44 - DNA, 681 bp, Artificial Sequence
  Description of Artificial SequenceN87D G53C cloned mutant chimeric murine 2D12.5 heavy chain variable region (VH) fused to human anti-tetanus toxin antibody CH1 heavy chain constant region (TTCH)
agatctgtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagttgtggaggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 45 - DNA, 681 bp, Artificial Sequence
  Description of Artificial SequenceN87D G54C cloned mutant chimeric murine 2D12.5 heavy chain variable region (VH) fused to human anti-tetanus toxin antibody CH1 heavy chain constant region (TTCH)
agatctgtgaagctgcaggagtctgggcctggcctagtgcagccctcacagagcctgtccatcacctgcacggtctctggtttctcattaactgactatggtgtacactgggttcgccagtctccaggaaagggtctggaatggctgggagtgatatggagtggttgtggcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 46 - DNA, 681 bp, Artificial Sequence
  Description of Artificial SequenceN87D G55C cloned mutant chimeric murine 2D12.5 heavy chain variable region (VH) fused to human anti-tetanus toxin antibody CH1 heavy chain constant region (TTCH)
agatctgtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggatgcacggcctatactgcggcgttcatatccagactgaacatctacaaggacaattccaagaaccaagttttctttgaaatgaacagtctgcaagctgatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcttggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 47 - DNA, 681 bp, Artificial Sequence
  Description of Artificial Sequencechimeric murine 2D12.5 heavy chain variable region (VH) fused to human anti-tetanus toxin antibody CH1 heavy chain constant region (TTCH) expected sequence
agatctgtgaagctgcaggagtctggacctggcctagtgcagccctcacagagcctgtccatcacctgcacggtctctggtttctcattaactgactatggtgtacactgggttcgccagtctccaggaaagggtctggaatggctgggagtgatatggagtggtggaggcacggcctatactgcggcgttcatatccagactgaacatctacaaggacaattccaagaaccaagttttctttgaaatgaacagtctgcaagctaatgacacagccatgtattactgtgccagaaggggtagctacccttacaactacttcgatgtctggggccaagggaccacggtcaccgtctccgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaatctaga

SEQ ID NO: 48 - DNA, 354 bp, Mus musculus; identical to SEQ ID NO: 15
  native hybridoma murine 2D12.5 heavy chain variable region (VH)

SEQ ID NO: 49 - DNA, 31 bp, Artificial Sequence
  Description of Artificial SequenceVH2D5′ BglII primer
gaagatctgtgaagctgcaggagtctggacc

SEQ ID NO: 50 - DNA, 35 bp, Artificial Sequence
  Description of Artificial SequenceCHTT2DMi primer
ccacggtcaccgtctccgcagcctccaccaagggc

SEQ ID NO: 51 - DNA, 35 bp, Artificial Sequence
  Description of Artificial SequenceVH2DTTMi primer
gcccttggtggaggctgcggagacggtgaccgtgg

SEQ ID NO: 52 - DNA, 33 bp, Artificial Sequence
  Description of Artificial SequenceTTCH3′ Xba primer
cgatctagatttgtcacaagatttgggctctgc

SEQ ID NO: 53 - DNA, 140 bp, Artificial Sequence
  Description of Artificial Sequenceheavy chain chimeric construct PCR reaction MT-VENT
gccaatgtgcatcagttgtggtcagcagcaaaatcaagtgaatcatctcagtgcaactaaaggggggatccgatctcaatatgaagttatgcatattactggccgtcgtcgcctttgttggcctctcgctcgggagatct

SEQ ID NO: 54 - protein, 18 aa, Artificial Sequence
  Description of Artificial SequenceBiP signal sequence
MKLCILLAVVAFVGLSLG

SEQ ID NO: 55 - DNA, 140 bp, Artificial Sequence
  Description of Artificial Sequenceheavy chain chimeric construct PCR reaction MT-VENT
tctagagggcccttcgaaggtaagcctatccctaaccctctcctcggtctcgattctacgcgtaccggtcatcatcaccatcaccattgagtttaaacccgctgatcagcctcgactgtgccttctaaggcctgagctcg

SEQ ID NO: 56 - protein, 23 aa, Artificial Sequence
  Description of Artificial SequenceV5 epitope and polyhistidine region
GKPIPNPLLGLDSTRTGHHHHHH

SEQ ID NO: 57 - DNA, 18 bp, Artificial Sequence
  Description of Artificial SequenceMTforward sequencing primer
catctcagtgcaactaaa

SEQ ID NO: 58 - DNA, 31 bp, Artificial Sequence
  Description of Artificial Sequence 2dvhN87D_pMTBip primer
catggctgtgtcatcagcttgcagactgttc

SEQ ID NO: 59 - DNA, 24 bp, Artificial Sequence
  Description of Artificial SequenceG53C noncoding 2dG53c_pMT primer
cgtgcctccacaactccatatcac

SEQ ID NO: 60 - DNA, 23 bp, Artificial Sequence
  Description of Artificial SequenceG54C noncoding 2dG54c_pMT primer
ccgtgccacaaccactccatatc

SEQ ID NO: 61 - DNA, 23 bp, Artificial Sequence
  Description of Artificial SequenceG55C noncoding 2dG55c_pMT primer
ccgtgcatccaccactccatatc

SEQ ID NO: 62 - DNA, 22 bp, Artificial Sequence
  Description of Artificial Sequence2dvhKBglII_pMTBip primer
gctcgggagatgtgtgaagctg

SEQ ID NO: 63 - DNA, 18 bp, Artificial Sequence
  Description of Artificial SequenceBGHreverse sequencing primer
tagaaggcacagtcgagg

SEQ ID NO: 64 - DNA, 29 bp, Artificial Sequence
  Description of Artificial Sequence2dVL5′Bgl primer
gaagatctgctgttgtgactcaggaatct

SEQ ID NO: 65 - DNA, 42 bp, Artificial Sequence
  Description of Artificial Sequence2dVLMiTop primer
accaaactgactgtcctaagccgaactgtggctgcaccatct

SEQ ID NO: 66 - DNA, 42 bp, Artificial Sequence
  Description of Artificial Sequence2dVLMiBot primer
agatggtgcagccacagttcggcttaggacagtcagtttggt

SEQ ID NO: 67 - DNA, 26 bp, Artificial Sequence
  Description of Artificial SequenceTTCL3′ Xba primer
cgatctagaattaacactctcccctg

SEQ ID NO: 68 - DNA, 138 bp, Artificial Sequence
  Description of Artificial Sequencelight chain chimeric construct PCR reaction MT-VENT
tagagggcccttcgaaggtaagcctatccctaaccctctcctcggtctcgattctacgcgtaccggtcatcatcaccatcaccattgagtttaaacccgctgatcagcctcgactgtgccttctaaggcctgagctcg

SEQ ID NO: 69 - DNA, 25 bp, Artificial Sequence
  Description of Artificial Sequence2dvL_S48G_pMTBip primer
cacctattagaccagtgaataaatg

SEQ ID NO: 70 - DNA, 38 bp, Artificial Sequence
  Description of Artificial SequenceS48G_N53C_pMTBip primer
cggttattacaaccacctattagaccagtgaataaatg

SEQ ID NO: 71 - DNA, 22 bp, Artificial Sequence
  Description of Artificial Sequence2dvLKBglII_pMTBip primer
gctcgggagatgtgctgttgtg

SEQ ID NO: 72 - protein, 12 aa, Artificial Sequence
  Description of Artificial Sequenceflexible linker
GGGSGGGSGGGS